(19) United States
(12) Patent Application Publication
(10) Pub. No.: US 2026/0347771 A1
(43) Pub. Date: Nov. 5, 2026

(54) COMPOSITIONS AND METHODS FOR ESTABLISHING SALIVARY GLAND ORGANOID CULTURES

(71) Applicant: UNIVERSITY OF WASHINGTON, Seattle, WA (US)

(72) Inventors: Devon EHNES, Seattle, WA (US); Hannele RUOHOLA-BAKER, Seattle, WA (US); Hee Yun JUNG, Seattle, WA (US); Ammar ALGHADEER, Seattle, WA (US); Sesha HANSON-DRURY, Seattle, WA (US); Julie MATHIEU, Seattle, WA (US)

(73) Assignee: UNIVERSITY OF WASHINGTON, Seattle, WA (US)

(21) Appl. No.: 19/490,203

(22) PCT Filed: Jun. 7, 2024
(86) PCT No.: PCT/US2024/033075

§ 371 Date: Dec. 4, 2025

Related U.S. Application Data
(60) Provisional application No. 63/472,140, filed on Jun. 9, 2023.

(51) Int. Cl. C12N 5/071; C12N 9/26; G01N 33/50

(52) U.S. Cl. CPC C12N 5/0633; C12N 9/2414; C12Y 302/01001; G01N 33/5088; C12N 2500/05; C12N 2500/24; C12N 2500/46; C12N 2501/11; C12N 2501/113; C12N 2501/117; C12N 2501/119; C12N 2501/13; C12N 2501/155; C12N 2501/33; C12N 2501/392; C12N 2501/415; C12N 2501/50; C12N 2501/727; C12N 2506/45; C12N 2533/52; C12N 2533/54

(57) ABSTRACT

The technology described herein is directed to compositions and methods for establishing salivary gland organoid culture. Such salivary gland organoid cultures are derived in vitro from pluripotent stem cells, such as induced pluripotent stems. Also described herein are uses for the salivary gland organoid cultures, including as a model for a salivary gland-associated disease or to screen for an effective therapeutic for a salivary gland-associated disease.

### CROSS-REFERENCE TO RELATED APPLICATIONS

This application claims benefit under 35 U.S.C. § 119(e) of U.S. Provisional Application No. 63/472,140 filed Jun. 9, 2023, the contents of which are incorporated herein by reference in their entirety.

### GOVERNMENT SUPPORT

This invention was made with government support under Grant No. T90 DE021984, awarded by the National Institutes of Health (NIH). The government has certain rights in the invention.

### TECHNICAL FIELD

The technology described herein relates to compositions and methods for establishing salivary gland organoid cultures.

### BACKGROUND

The salivary gland is an exocrine gland that produces and secretes saliva. Saliva plays roles in tissue repair, oral lubrication, tooth mineralization and protection, and taste. Several factors can perturb the proper function of salivary glands, including Sjögren's syndrome, as well as cancer and resultant radiation therapy. Compounding this issue is that salivary glands have been shown to have poor regenerative capacity following injury despite reports of the existence of stem-like cells in adult tissues. Salivary gland development and function in vivo is complex. There is great need for in vitro salivary gland models.

### SUMMARY

The technology described herein is directed to compositions and methods for establishing salivary gland organoid cultures. Such salivary gland organoid cultures are derived in vitro from pluripotent stem cells, such as induced pluripotent stems. Also described herein are uses for the salivary gland organoid cultures, including as a model for a salivary gland-associated disease or to screen for an effective therapeutic for a salivary gland-associated disease.

Accordingly, in one aspect, described herein is a composition comprising a salivary gland organoid culture.

In some embodiments of any of the aspects, the salivary gland organoid culture comprises salivary gland epithelial cells.

In some embodiments of any of the aspects, the salivary gland epithelial cells express acinar marker Amylase Alpha 1A (AMY1A), duct marker keratin 19 (KRT19), myoepithelial marker Actin Alpha 2 (ACTA2), and/or neuroepithelium marker Tubulin Beta 3 Class III (TUBB3).

In some embodiments of any of the aspects, the salivary gland organoid culture comprises salivary gland buds, branches, and/or lumenized ducts.

In some embodiments of any of the aspects, the salivary gland organoid culture is substantially free of endothelium and mesenchyme.

In some embodiments of any of the aspects, the salivary gland organoid culture is derived from pluripotent stem cells.

In some embodiments of any of the aspects, the salivary gland organoid culture can produce saliva.

In some embodiments of any of the aspects, the composition further comprises a salivary gland organoid culture medium.

In some embodiments of any of the aspects, the salivary gland organoid medium comprises: (a) a medium supplement comprising: (i) transferrin; (ii) insulin: (iii) progesterone; (iv) putrescine; and/or (v) selenite; (b) at least one fibroblast growth factor; (c) a neuregulin; (d) an extracellular matrix glycoprotein; (e) a basement membrane matrix: (f) a growth factor for epithelium; (g) an inhibitor of a Glycogen Synthase Kinase (GSK) and/or activator of a Wingless/Integrated (Wnt) and β-catenin pathway; and/or (h) an inhibitor of transforming growth factor-β (TGF-β) type I receptor.

In one aspect, described herein is a method of establishing a salivary gland organoid culture, the method comprising: (a) culturing a population of pluripotent stem cells in aggregation medium for a sufficient time to promote formation of phripotent stem cell clusters; (b) culturing the resultant pluripotent stem cell clusters in a salivary epithelium differentiation medium for a sufficient time to promote differentiation into salivary epithelium; and (c) culturing the resultant salivary epithelium in a salivary gland organoid differentiation medium for a sufficient time to promote differentiation into a salivary gland organoid culture.

In some embodiments of any of the aspects, the pluripotent stem cells comprise induced pluripotent stem cells (iPSCs).

In some embodiments of any of the aspects, the iPSCs are derived from a somatic cell sample from a subject.

In some embodiments of any of the aspects, the iPS cell is derived from a subject with a disease or disorder that affects salivary gland function.

In some embodiments of any of the aspects, the iPSCs comprise cell line WTC-11.

In some embodiments of any of the aspects, the aggregation medium comprises an inhibitor of Rho Associated Coiled-Coil Containing Protein Kinase (ROCK).

In some embodiments of any of the aspects, the inhibitor of ROCK is Y-27632.

In some embodiments of any of the aspects, the inhibitor of ROCK is at a concentration of about 50 μM.

In some embodiments of any of the aspects, the aggregation medium comprises mTeSR™I (STEM CELL TECHNOLOGIES) basal medium, which can be used for the maintenance of human pluripotent stem cells.

In some embodiments of any of the aspects, the sufficient amount of time to promote formation of pluripotent stem cell clusters is about 3 days.

In some embodiments of any of the aspects, the salivary epithelium differentiation medium comprises at least one of the following: (a) an agonist of Smoothened from about day 0 to about day 8 after beginning the salivary epithelium differentiation medium: (b) a bone morphogenetic protein from about day 3 to about day 8 after beginning the salivary epithelium differentiation medium; (c) from about day 8 to about day 12 after beginning the salivary epithelium differentiation medium: (i) an agonist of Smoothened; (ii) an inhibitor of a Glycogen Synthase Kinase (GSK) and/or activator of a Wingless/Integrated (Wnt) and β-catenin pathway; (iii) a growth factor for epithelium; (iv) an inhibitor of a Bone Morphogenetic Protein type I (BMP I) receptor; and/or (v) a neurotrophin; and/or (d) a fibroblast growth factor from about day 10 to about day 12 after beginning the salivary epithelium differentiation medium.

In some embodiments of any of the aspects, the salivary epithelium differentiation medium comprises at least one of the following: (a) an agonist of Smoothened at a concentration of about 400 nM from about day 0 to about day 8 after beginning the salivary epithelium differentiation medium; (b) a bone morphogenetic protein at a concentration of about 150 μM from about day 3 to about day 8 after beginning the salivary epithelium differentiation medium; (c) from about day 8 to about day 12 after beginning the salivary epithelium differentiation medium: (i) an agonist of Smoothened at a concentration of about 1 mM; (ii) an inhibitor of a Glycogen Synthase Kinase (GSK) and/or activator of a Wingless/Integrated (Wnt) and β-catenin pathway at a concentration of about 5 μM; (iii) a growth factor for epithelium at a concentration of about 15 ng/mL; (iv) an inhibitor of a Bone Morphogenetic Protein type I (BMP I) receptor at a concentration of about 1 μM; and/or (v) a neurotrophin at a concentration of about 1 μM; and/or (d) a fibroblast growth factor at a concentration of about 250 ng/mL from about day 10 to about day 12 after beginning the salivary epithelium differentiation medium.

In some embodiments of any of the aspects, (a) the agonist of Smoothened is Smoothened Agonist (SAG); (b) the bone morphogenetic protein is bone morphogenetic protein 4 (BMP4); (c) the inhibitor of GSK and/or activator of the Wnt and β-catenin pathway is Chiron (Chir99021); (d) the growth factor for epithelium is epidermal growth factor (EGF): (e) the inhibitor of the BMP type 1 receptor is LDN-193189; (f) the neurotrophin is Neurotrophin-4; and/or (g) the fibroblast growth factor is fibroblast growth factor 10 (FGF10).

In some embodiments of any of the aspects, the salivary epithelium differentiation medium comprises at least one of the following: (a) Smoothened Agonist (SAG) at a concentration of about 400 nM from about day 0 to about day 8 after beginning the salivary epithelium differentiation medium; (b) bone morphogenetic protein 4 (BMP4) at a concentration of about 150 μM from about day 3 to about day 8 after beginning the salivary epithelium differentiation medium: (c) from about day 8 to about day 12 after beginning the salivary epithelium differentiation medium: (i) SAG at a concentration of about 1 mM; (ii) Chiron (Chir99021) at a concentration of about 5 μM: (iii) epidermal growth factor (EGF) at a concentration of about 15 ng/mL; (iv) an inhibitor of a Bone Morphogenetic Protein type I receptor (BMP 1) LDN-193189 at a concentration of about 1 μM; and/or (v) Neurotrophin-4 at a concentration of about 1 μM; and/or (d) fibroblast growth factor 10 (FGF10) at a concentration of about 250 ng/mL from about day 10 to about day 12 after beginning the salivary epithelium differentiation medium.

In some embodiments of any of the aspects, the salivary epithelium differentiation medium comprises EPICULT-C™ medium.

In some embodiments of any of the aspects, the sufficient amount of time to promote differentiation of pluripotent stem cell clusters into salivary epithelium is about 12 days.

In some embodiments of any of the aspects, the salivary gland organoid differentiation medium comprises at least one of the following: (a) from about day 12 to about day 35-75 after beginning the salivary epithelium differentiation medium (or from about day 0 to about day 23-63 after beginning the salivary gland organoid differentiation medium): (i) a medium supplement comprising: (A) transferrin; (B) insulin: (C) progesterone; (D) putrescine; and/or (E) selenite; and/or (ii) at least one fibroblast growth factor: (b) a neuregulin from about day 18 to about day 35-75 after beginning the salivary epithelium differentiation medium (or from about day 6 to about day 23-63 after beginning the salivary gland organoid differentiation medium); (c) from about day 18 to about day 24-75 after beginning the salivary epithelium differentiation medium (or from about day 6 to about day 12-63 after beginning the salivary gland organoid differentiation medium): (i) an extracellular matrix glycoprotein; and/or (ii) a basement membrane matrix: (d) an inhibitor of Rho Associated Coiled-Coil Containing Protein Kinase (ROCK) from about day 24 to about day 27 after beginning the salivary epithelium differentiation medium (or from about day 12 to about day 15 after beginning the salivary gland organoid differentiation medium); and/or (e) from about day 24 to about day 35-75 after beginning the salivary epithelium differentiation medium (or from about day 12 to about day 23-63 after beginning the salivary gland organoid differentiation medium): (i) a growth factor for epithelium: (ii) an inhibitor of a Glycogen Synthase Kinase (GSK) and/or activator of a Wingless/Integrated (Wnt) and β-catenin pathway; and/or (iii) an inhibitor of transforming growth factor-β (TGF-β) type I receptor.

In some embodiments of any of the aspects, the salivary gland organoid differentiation medium comprises at least one of the following: (a) from about day 12 to about day 35-75 after beginning the salivary epithelium differentiation medium (or from about day 0 to about day 23-63 after beginning the salivary gland organoid differentiation medium): (i) a medium supplement (e.g., at a 1× concentration in the salivary gland organoid differentiation medium) comprising: (A) transferrin at a concentration of about 10 mg/L; (B) insulin at a concentration of about 50 mg/L; (C) progesterone at a concentration of about 6.3 μg/L; (D) putrescine at a concentration of about 16.11 mg/L; and/or (E) selenite at a concentration of about 5.2 μg/L; and/or (ii) at least one fibroblast growth factor at a concentration from about 125 ng/mL to about 250 ng/mL; (b) a neuregulin at a concentration of about 250 ng/mL from about day 18 to about day 35-75 after beginning the salivary epithelium differentiation medium (or from about day 6 to about day 23-63 after beginning the salivary gland organoid differentiation medium): (c) from about day 18 to about day 24-75 after beginning the salivary epithelium differentiation medium (or from about day 6 to about day 12-63 after beginning the salivary gland organoid differentiation medium): (i) an extracellular matrix glycoprotein at a concentration of about 1%; and/or (ii) a basement membrane matrix at a concentration of about 3%; (d) an inhibitor of Rho Associated Coiled-Coil Containing Protein Kinase (ROCK) at a concentration of about 50 μM from about day 24 to about day 27 after beginning the salivary epithelium differentiation medium (or from about day 12 to about day 15 after beginning the salivary gland organoid differentiation medium); and/or (e) from about day 24 to about day 35-75 after beginning the salivary epithelium differentiation medium (or from about day 12 to about day 23-63 after beginning the salivary gland organoid differentiation medium): (i) a growth factor for epithelium at a concentration of about 100 ng/mL; (ii) an inhibitor of a Glycogen Synthase Kinase (GSK) and/or activator of a Wingless/Integrated (Wnt) and β-catenin pathway at a concentration of about 5 μM; and/or (iii) an inhibitor of transforming growth factor-β (TGF-β) type I receptor at a concentration of about 10 μM.

In some embodiments of any of the aspects, the salivary gland organoid differentiation medium comprises at least one of the following: (a) from about day 12 to about day 35-75 after beginning the salivary epithelium differentiation medium (or from about day 0 to about day 23-63 after beginning the salivary gland organoid differentiation medium): (i) a medium supplement (e.g., at a 100× concentration before being diluted to 1× in the salivary gland organoid differentiation medium) comprising: (A) transferrin at a concentration of about 1000 mg/L; (B) insulin at a concentration of about 500 mg/L: (C) progesterone at a concentration of about 0.63 mg/L; (D) putrescine at a concentration of about 1611 mg/L; and/or (E) selenite at a concentration of about 0.52 mg/L; and/or (ii) at least one fibroblast growth factor at a concentration from about 125 ng/mL to about 250 ng/mL; (b) a neuregulin at a concentration of about 250 ng/mL from about day 18 to about day 35-75 after beginning the salivary epithelium differentiation medium (or from about day 6 to about day 23-63 after beginning the salivary gland organoid differentiation medium); (c) from about day 18 to about day 24-75 after beginning the salivary epithelium differentiation medium (or from about day 6 to about day 12-63 after beginning the salivary gland organoid differentiation medium): (i) an extracellular matrix glycoprotein at a concentration of about 1%; and/or (ii) a basement membrane matrix at a concentration of about 3%; (d) an inhibitor of Rho Associated Coiled-Coil Containing Protein Kinase (ROCK) at a concentration of about 50 μM from about day 24 to about day 27 after beginning the salivary epithelium differentiation medium (or from about day 12 to about day 15 after beginning the salivary gland organoid differentiation medium); and/or (e) from about day 24 to about day 35-75 after beginning the salivary epithelium differentiation medium (or from about day 12 to about day 23-63 after beginning the salivary gland organoid differentiation medium): (i) a growth factor for epithelium at a concentration of about 100 ng/mL; (ii) an inhibitor of a Glycogen Synthase Kinase (GSK) and/or activator of a Wingless/Integrated (Wnt) and β-catenin pathway at a concentration of about 5 μM; and/or (iii) an inhibitor of transforming growth factor-β (TGF-β) type I receptor at a concentration of about 10 μM.

In some embodiments of any of the aspects, (a) the media supplement is N-2 supplement: (b) the neuregulin is Neuregulin 1 (NRG1); (c) the at least one fibroblast growth factor is selected from the group consisting of: fibroblast growth factor 10 (FGF10), fibroblast growth factor 7 (FGF7), and fibroblast growth factor 1 (FGF1); (d) the extracellular matrix glycoprotein is a laminin; (e) the basement membrane matrix is secreted by Engelbreth-Hohn-Swarm (EHS) mouse sarcoma cells (MATRIGEL®); (f) the inhibitor of ROCK is Y-27632; (g) the growth factor for epithelium is epidermal growth factor (EGF); (h) the inhibitor of GSK and/or activator of the Wnt and β-catenin pathway is Chiron (Chir99021); and/or (i) the inhibitor of the TGF-β type I receptor is SB-431452.

In some embodiments of any of the aspects, the basement membrane matrix is growth factor reduced (GFR).

In some embodiments of any of the aspects, the salivary gland organoid differentiation medium comprises at least one of the following: (a) from about day 12 to about day 35-75 after beginning the salivary epithelium differentiation medium (or from about day 0 to about day 23-63 after beginning the salivary gland organoid differentiation medium): (i) an N-2 medium supplement (e.g., at a 1× concentration in the salivary gland organoid differentiation medium) comprising: (A) transferrin at a concentration of about 10 mg/L; (B) insulin at a concentration of about 50 mg/L; (C) progesterone at a concentration of about 6.3 μg/L: (D) putrescine at a concentration of about 16.11 mg/L; and/or (E) selenite at a concentration of about 5.2 μg/L; (ii) fibroblast growth factor 10 (FGF10) at a concentration of about 250 ng/mL; (iii) fibroblast growth factor 7 (FGF7) at a concentration of about 125 ng/mL; and/or (iv) fibroblast growth factor I (FGF1) at a concentration of about 125 ng/mL; (b) NRG1 at a concentration of about 250 ng/mL from about day 18 to about day 35-75 after beginning the salivary epithelium differentiation medium (or from about day 6 to about day 23-63 after beginning the salivary gland organoid differentiation medium); (c) from about day 18 to about day 24-75 after beginning the salivary epithelium differentiation medium (or from about day 6 to about day 12-63 after beginning the salivary gland organoid differentiation medium): (i) laminin at a concentration of about 1%; and/or (ii) MATRIGEL® at a concentration of about 3%; (d) Y-27632 at a concentration of about 50 μM from about day 24 to about day 27 after beginning the salivary epithelium differentiation medium (or from about day 12 to about day 15 after beginning the salivary gland organoid differentiation medium); and/or (e) from about day 24 to about day 35-75 after beginning the salivary epithelium differentiation medium (or from about day 12 to about day 23-63 after beginning the salivary gland organoid differentiation medium): (i) EGF at a concentration of about 100 ng/mL; (ii) Chiron at a concentration of about 5 μM; and/or (iii) SB-431452 at a concentration of about 10 μM.

In some embodiments of any of the aspects, the salivary gland organoid differentiation medium comprises at least one of the following: (a) from about day 12 to about day 35-75 after beginning the salivary epithelium differentiation medium (or from about day 0 to about day 23-63 after beginning the salivary gland organoid differentiation medium): (i) an N-2 medium supplement (e.g., at a 100× concentration before being diluted to 1× in the salivary gland organoid differentiation medium) comprising: (A) transferrin at a concentration of about 1000 mg/L; (B) insulin at a concentration of about 500 mg/L: (C) progesterone at a concentration of about 0.63 mg/L; (D) putrescine at a concentration of about 1611 mg/L; and/or (E) selenite at a concentration of about 0.52 mg/L: (ii) fibroblast growth factor 10 (FGF10) at a concentration of about 250 ng/mL; (iii) fibroblast growth factor 7 (FGF7) at a concentration of about 125 ng/mL; and/or (iv) fibroblast growth factor 1 (FGF1) at a concentration of about 125 ng/mL; (b) NRG1 at a concentration of about 250 ng/mL from about day 18 to about day 35-75 after beginning the salivary epithelium differentiation medium (or from about day 6 to about day 23-63 after beginning the salivary gland organoid differentiation medium); (c) from about day 18 to about day 24-75 after beginning the salivary epithelium differentiation medium (or from about day 6 to about day 12-63 after beginning the salivary gland organoid differentiation medium): (i) laminin at a concentration of about 1%; and/or (ii) MATRIGEL® at a concentration of about 3%; (d) Y-27632 at a concentration of about 50 μM from about day 24 to about day 27 after beginning the salivary epithelium differentiation medium (or from about day 12 to about day 15 after beginning the salivary gland organoid differentiation medium); and/or (e) from about day 24 to about day 35-75 after beginning the salivary epithelium differentiation medium (or from about day 12 to about day 23-63 after beginning the salivary gland organoid differentiation medium): (i) EGF at a concentration of about 100 ng/mL; (ii) Chiron at a concentration of about 5 μM; and/or (iii) SB-431452 at a concentration of about 10 μM.

In some embodiments of any of the aspects, the salivary gland organoid differentiation medium comprises Dulbecco's Modified Eagle Medium and Nutrient Mixture F12 (DMEM/F12™) medium.

In some embodiments of any of the aspects, the sufficient amount of time to promote differentiation of salivary epithelium into the salivary gland organoid culture is about 23-63 days.

In some embodiments of any of the aspects, the sufficient amount of time to promote differentiation of salivary epithelium into the salivary gland organoid culture is about 38 days.

In some embodiments of any of the aspects, the sufficient amount of time to promote differentiation of the population of pluripotent stem cells into the salivary gland organoid culture is about 53 days.

In some embodiments of any of the aspects, the sufficient amount of time to promote differentiation of the pluripotent stem cell clusters into the salivary gland organoid culture is about 50 days.

In some embodiments of any of the aspects, the sufficient amount of time to promote differentiation of the pluripotent stem cell clusters into the salivary gland organoid culture is about 30 days to 50 days.

In some embodiments of any of the aspects, the salivary gland organoid culture secretes salivary amylase after about day 35 after beginning the salivary epithelium differentiation medium.

In some embodiments of any of the aspects, the salivary gland organoid differentiation medium further comprises from at least about day 35 after beginning the salivary epithelium differentiation medium (or from at least about day 27 after beginning the salivary gland organoid differentiation medium): (a) a sugar or sugar alcohol; and/or (b) at least one proinflammatory cytokine.

In some embodiments of any of the aspects, the salivary gland organoid differentiation medium further comprises from at least about day 35 after beginning the salivary epithelium differentiation medium (or from at least about day 27 after beginning the salivary gland organoid differentiation medium): (a) a sugar or sugar alcohol at a concentration of about 53-70 mM; and/or (b) at least one proinflammatory cytokine at a concentration of about 1 ng/ml.

In some embodiments of any of the aspects, (a) the sugar is D-glucose; (b) the sugar alcohol is D-mannitol; and/or (c) the at least one proinflammatory cytokine is TNFα and/or IL-6.

In some embodiments of any of the aspects, the salivary gland organoid differentiation medium further comprises from at least about day 35 after beginning the salivary epithelium differentiation medium (or from at least about day 27 after beginning the salivary gland organoid differentiation medium): (a) D-glucose or D-mannitol at a concentration of about 53-70 mM; and/or (b) TNFα and/or IL-6 at a concentration of about 1 ng/ml.

In one aspect, described herein is a salivary gland organoid culture prepared according to the methods as described herein.

In one aspect, described herein is a salivary gland organoid culture derived from pluripotent stem cells.

In one aspect, described herein is a salivary gland organoid culture as described herein, implanted into the kidney capsule of a subject.

In one aspect, described herein is a use of the salivary gland organoid culture as described herein as a model for a salivary gland-associated disease.

In one aspect, described herein is a use of the salivary gland organoid culture as described herein to screen for an effective therapeutic for a salivary gland-associated disease.

In some embodiments of any of the aspects, the salivary gland-associated disease is selected from the group consisting of: (a) a metabolic disease; (b) an inflammatory disease; (c) an autoimmune disease; and/or (d) radiation damage.

In some embodiments of any of the aspects, (a) the metabolic disease is type 2 diabetes: (b) the inflammatory disease is Sjögren's syndrome; (c) the autoimmune disease is type 1 diabetes; and/or (d) the radiation damage is from radiation treatment for head or neck cancer.

In some embodiments of any of the aspects, the salivary gland organoid culture is implanted into the kidney capsule of a subject.

In one aspect, described herein is a method of screening for an effective therapeutic for a salivary gland-associated disease, the method comprising: (a) exposing the salivary gland organoid culture as described herein to a potential therapeutic for a salivary gland-associated disease; (b) determining whether the salivary gland organoid culture exhibits decreased levels of a disease-associated marker for the salivary gland-associated disease or increased levels of a health-associated marker compared to the salivary gland organoid culture not exposed to the potential therapeutic; and (c) determining that the potential therapeutic is an effective therapeutic for the salivary gland-associated disease if the salivary gland organoid culture exhibits decreased levels of a disease-associated marker for the salivary gland-associated disease or increased levels of a health-associated marker compared to the salivary gland organoid culture not exposed to the potential therapeutic; or (d) determining that the potential therapeutic is not an effective therapeutic for the salivary gland-associated disease if the salivary gland organoid culture did not exhibit decreased levels of a disease-associated marker for the salivary gland-associated disease or increased levels of a health-associated marker compared to the salivary gland organoid culture not exposed to the potential therapeutic.

In some embodiments of any of the aspects, the health-associated marker is selected from the group consisting of Amylase Alpha 1A (AMY1A), keratin 19 (KRT19), and Actin Alpha 2 (ACTA2).

### BRIEF DESCRIPTION OF THE DRAWINGS

FIG. 1A-1F: Cell clustering and pseudotime analysis reveal four distinct developmental trajectories for major tissues in the salivary gland. (FIG. 1A) Salivary gland development begins in humans at 6 weeks gestation and goes through several stages, the latter of which are the pseudoglandular stage and the canalicular stage. Fully mature salivary glands (not observed until after birth) exhibit 5 distinct types of adult cells: the functional acinar cell, the ID, the SD, the excretory duct, and the myoepithelial cell. (FIG. 1B) Human fetal tissue at 12 weeks resemble early pseudoglandular stage, wherein some limited branching can be observed and ducts are mostly non-humanized or have small lumens. From 14-16w, there are more obvious bifurcations and larger lumens. By 17-19w most ducts exhibit some kind of lumen and widespread branching is obvious. (FIG. 1C) Sci-sequencing and unbiased clustering of submandibular human salivary gland tissue from 12-19w gestation yielded 16 clusters. The salivary epithelium (left box comprising groups (a), (b), (c), (d), (g), (h), (i), (j), (k)) grouped along an obvious separation from support tissues (right box comprising groups (e), (f), (l), (in), (n)). Clusters of cells were identified from each ductal type using previously specified markers or functional markers, and though the tissue was yet too immature to have acinar cells, a small subpopulation of cells was observed that expressed genes associated with acinar identity, which were denoted as proacinar cells. (FIG. 1D) To facilitate trajectory analysis. the dataset was subset to only include the clusters associated with salivary epithelium (from the left box in FIG. 1C), resulting in 9 clusters. (FIG. 1E) Bioinformatic trajectory prediction and pseudotime analysis indicate that salivary gland development begins with a population of BEPs (Cluster 7) and over several bifurcations, yields distinct trajectories (FIG. 1F).

FIG. 2A-21: Bifurcation of ductal progenitors relies on differential regulation of secreted signals and cell-cell interaction. (FIG. 2A) Top gene expression analysis of striated ducts (SDs) and intercalated ducts (IDs) show that duct progenitors (DPs) share transcriptional overlap with both SD and ID, but much more so with ID. (FIG. 2B) Differential expression and ChEA3 analysis and ligand-receptor analysis (FIG. 2D-2F) indicate that TGFβ signaling can promote striated identity while Wnt promotes intercalated identity. Extracellular matrix (ECM)-cell interaction analysis (FIG. 2C-2H) indicates that interaction between laminin and different integrins can also contribute to this bifurcation. Analysis indicates that ELF3 and ELF5 mark intercalated and SDs respectively, and that TGFB2 from ID helps to drive TGFβ signaling in SD (FIG. 21).

FIG. 3A-3B: Bioinformatic analysis introduces new potential regulators of acinar differentiation. (FIG. 3A) MUC4, an acinar-associated mucin, was observed in tissues after 19w. (FIG. 3B) Transcriptional analysis shows that DT shares significant transcriptional overlap with ID, and some limited transcriptional overlap with proacinar cells. CHEA3 analysis based on top genes in each cluster show that TSHZ2 and EHF are responsible for transcription of many top expressed factors in the distal tip. while several AP-1 transcription factors drive top expression in the distal tip.

FIG. 4A-4W: sci-Seq-guided human induced pluripotent stem cell (hiPSC)-derived salivary gland organoids. (FIG. 4A) Salivary gland organoids are developed over a period of 53 days, going from iPSCs to oral epithelium and then toward salivary epithelium. During that time, organoids exhibit budding and branching, and by day 50 (FIG. 4B), they exhibit expression of acinar marker AMY1A, duct marker KRT19, and myoepithelial marker ACTA2. It also shows distinct organization of the salivary gland cell types, as well as lumenized duct like structures (arrows). By d27 (FIG. 4C), small vesicles were observed budding off the organoids, which are numerous by d50 (FIG. 4D), and which completely bud off and settle on the bottom of the well. Analysis of these vesicles in culture media via western blot demonstrated the presence of AMY1A, indicating they are secreting this critical salivary enzyme into their surroundings (see e.g., FIG. 5H). Additionally, by d50, organoids exhibit some expression of TUBB3, indicating that organoids can be manipulated to produce their own neuroepithelium (FIG. 4E). To evaluate viability and growth in vivo, organoids that had reached d35 were implanted in the kidney capsule of NOD SCID immune compromised mice (FIG. 4F), and they were allowed to continue to develop for 3 weeks, after which mice were sacrificed and kidneys harvested (FIG. 4G). There was robust survival of organoids, distinguishable from mouse tissue by expression of human nuclear antigen (HNA), as well as preservation of their 3D structure, and robust expression of AMY1A in the graft (FIG. 4H-41). Single cell combinatorial indexing RNA Seq of salivary gland organoids at d56 resulted in 5 clusters (FIG. 4J) with a distinct developmental trajectory (FIG. 4K), 4 of which exhibit KEGG analysis results consistent with functionality of cells in striated duct and acinar cells (Clusters 1-4, FIGS. 4L-O, respectively). Additionally, Cluster 1 expressed several salivary specific markers (FIG. 4P), including genes from the histatin (HST), cystatin (CST), and mucin (MUC) families. It also exhibited expression of some salivary gland enriched transporter proteins (Solute Carrier Family 12 Member 2 (SLC12A2), Solute Carrier Family 5 Member 5 (SLC5A5), Solute Carrier Family 26 Member 9 (SLC26A9)). Using bioinformatic tools to directly compare sequenced salivary gland organoids to the human fetal data presented in FIG. 1-3, it was found that cells from salivary gland organoids overlay aligned with cells from all clusters but were primarily localized to more immature cell types in the fetal data (FIG. 4Q), consistent with what is observed in organoid tissues at large. However, this shows that the organoids are progressing down a path of differentiation that has made them at least as mature as human fetal salivary gland tissue at 12 weeks gestational age, and more mature in some cases. Finally, to demonstrate functionality of these organoids, they were assayed for their ability to respond to the cholinergic agonist carbachol, mimicking the response produced by the facial nerve to stimulate saliva flow in vivo. Sham treatment with DMSO (FIG. 4R-4T) resulted in minimal signaling activity, while carbachol stimulation resulted in substantial stimulation (FIG. 4U-4W), indicating that these can be externally stimulated, like salivary glands to produce saliva.

FIG. 5A-5M: hiPSC salivary gland organoids as a diabetic model. After organoids begin to express AMY1a at d35 (FIG. 5A-5B) or d50 (FIG. 5C-5G), the organoids were exposed to osmotic control or 70 mM glucose with 1 ng/mL diabetic cytokines TNFa and IL-6 (FIG. 5B, 5F, 5G) for an additional 14 days. Diabetic conditions diminished expression of AMY1a, consistent with saliva in diabetic patients. Coomassie stain of control and diabetic culture media collected and concentrated showed an aggregation of protein at the same size as the positive control containing recombinant amylase (FIG. 5H). Western blot analysis (FIG. 5I) showed diminished presence of salivary amylase in diabetic media samples compared to controls. Bulk sequencing analysis showed that in diabetic samples, significantly upregulated genes (FIG. 5J) were limited and were associated with advanced glycation end products (AGE) and receptor for AGE (RAGE) signaling pathway, EGFR signaling, TGFbeta signaling, and other pathways commonly implicated in chronic diabetic and inflammatory environments. Significantly downregulated genes (FIG. 5K) were numerous and were associated with ribosomal function, oxidative phosphorylation, and proteasomal function, also consistent with phenotypes in other diabetic tissues. Seahorse Flux analysis was performed to measure the oxygen consumption to confirm that the defective oxidative phosphorylation was linked to mitochondrial stress (FIG. 5L, M). While there was a limited difference in the cells' ability to produce ATP, and the Spare Respiratory Capacity was unchanged, there were significant differences in both basal respiration as well as the cells' ability to reach maximal respiration when chemically provoked, indicating that the diabetic model recapitulates the mitochondrial dysfunction observed in many other diabetic systems.

### DETAILED DESCRIPTION

Embodiments of the technology described herein include compositions and methods for establishing salivary gland organoid cultures. Such salivary gland organoid cultures are derived in vitro from pluripotent stem cells, such as induced pluripotent stems. Also described herein are uses for the salivary gland organoid cultures, including as a model for a salivary gland-associated disease or to screen for an effective therapeutic for a salivary gland-associated disease.

### Salivary Gland Organoid Cultures

In multiple aspects, described herein are compositions comprising a salivary gland organoid culture. As used herein, the term “organoid” refers to a 3-dimensional growth of mammalian cells in culture that retains characteristics of the tissue in vivo, e.g. prolonged tissue expansion with proliferation, multilineage differentiation, and/or recapitulation of cellular and tissue ultrastructure, etc.

In multiple embodiments, the salivary gland organoid cultures described herein comprise salivary gland epithelial cells. In some embodiments, the salivary gland epithelial cells comprise acinar cells, duct cells, myoepithelial cells, and/or neuroepithelium. In some embodiments, at least one of the salivary gland epithelial cells in the salivary gland organoid culture expresses acinar marker Amylase Alpha 1A (AMY1A). In some embodiments, at least one of the salivary gland epithelial cells in the salivary gland organoid culture expresses duct marker keratin 19 (KRT19). In some embodiments, at least one of the salivary gland epithelial cells in the salivary gland organoid culture expresses myoepithelial marker Actin Alpha 2 (ACTA2). In some embodiments, at least one of the salivary gland epithelial cells in the salivary gland organoid culture expresses neuroepithelium marker Tubulin Beta 3 Class III (TUBB3).

In some embodiments, salivary gland epithelial cells in the salivary gland organoid culture express acinar marker Amylase Alpha 1A (AMY1A), duct marker keratin 19 (KRT19), myoepithelial marker Actin Alpha 2 (ACTA2), and neuroepithelium marker Tubulin Beta 3 Class III (TUBB3). Either the same or different individual salivary gland epithelial cells in the salivary gland organoid culture express each of the markers: AMY1A, KRT19, TUBB3, and/or ACTA2.

In some embodiments, the salivary gland organoid culture comprises salivary gland buds, branches, and/or lumenized ducts. In some embodiments, the salivary gland organoid culture comprises salivary gland buds. In some embodiments, the salivary gland organoid culture comprises salivary gland branches. In some embodiments, the salivary gland organoid culture comprises lumenized ducts. In some embodiments, the salivary gland organoid culture comprises salivary gland buds and branches. In some embodiments, the salivary gland organoid culture comprises salivary gland buds and lumenized ducts. In some embodiments, the salivary gland organoid culture comprises salivary gland branches and lunenized ducts. In some embodiments, the salivary gland organoid culture comprises salivary gland buds, branches, and lumenized ducts.

In some embodiments, the salivary gland organoid culture is substantially free of endothelium and/or mesenchyme. In some embodiments, the salivary gland organoid culture is substantially free of endothelium. In some embodiments, the salivary gland organoid culture is substantially free of mesenchyme. As used herein, the term “substantially free” refers to a state in which relatively little or no amount of an indicated substance is present. As a non-limiting example, “substantially free of endothelium and/or mesenchyme” means the salivary gland organoid culture contains less than approximately 5%, 4%, 3%, 2%, 1.0%, 0.9%, 0.8%, 0.7%, 0.6%, 0.5%, 0.4%, 0.3%, 0.2%, 0.1% or less (e.g., by cell number) of the endothelium and/or mesenchyme. In some embodiments, the presence of endothelium can be detected using endothelial cell markers, including but not limited to Von Willebrand Factor (VWF), Angiotensin I Converting Enzyme (ACE), P-selectin, or A Disintegrin And Metalloproteinase With Thrombospondin Motifs 13 (ADAMTS13). In some embodiments, the presence of mesenchyme can be detected using mesenchyme cell markers, including but not limited to CD73, CD90, or CD105.

In some embodiments, the salivary gland organoid culture is derived in culture from pluripotent stem cells. In some embodiments, the salivary gland organoid culture is not cultured from non-pluripotent stem cells present in or isolated or purified from a tissue sample.

In some embodiments, the salivary gland organoid culture is capable of producing saliva. In some embodiments, the salivary gland organoid culture is capable of producing saliva in response to a stimulus, such as a cholinergic agonist (e.g., carbachol).

In some embodiments, the composition comprising the salivary gland organoid culture further comprises a salivary gland organoid culture medium. Non-limiting examples of such salivary gland organoid culture media are described herein. In some embodiments, the salivary gland organoid culture is prepared according to one of the methods described further herein.

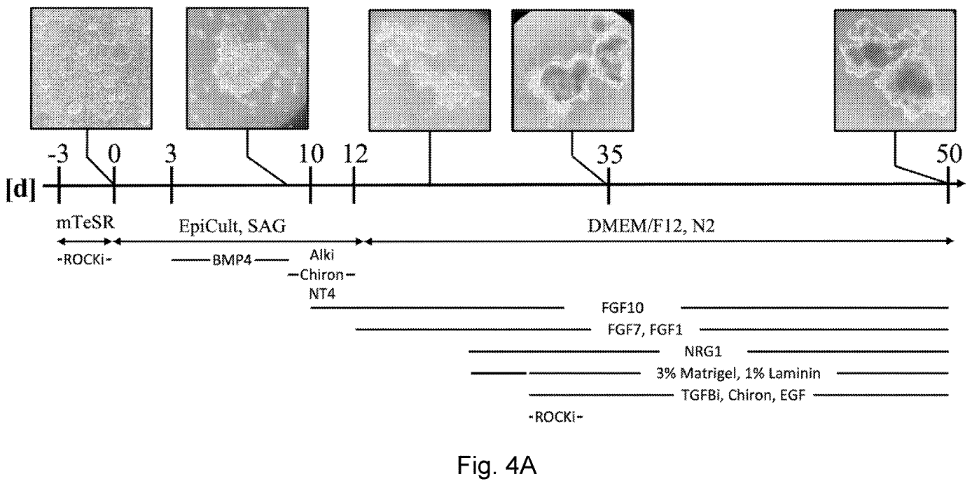

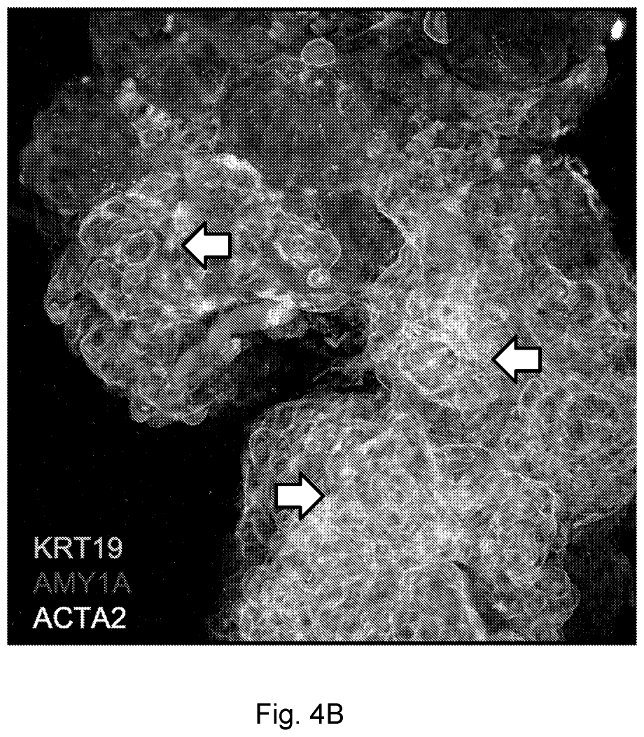

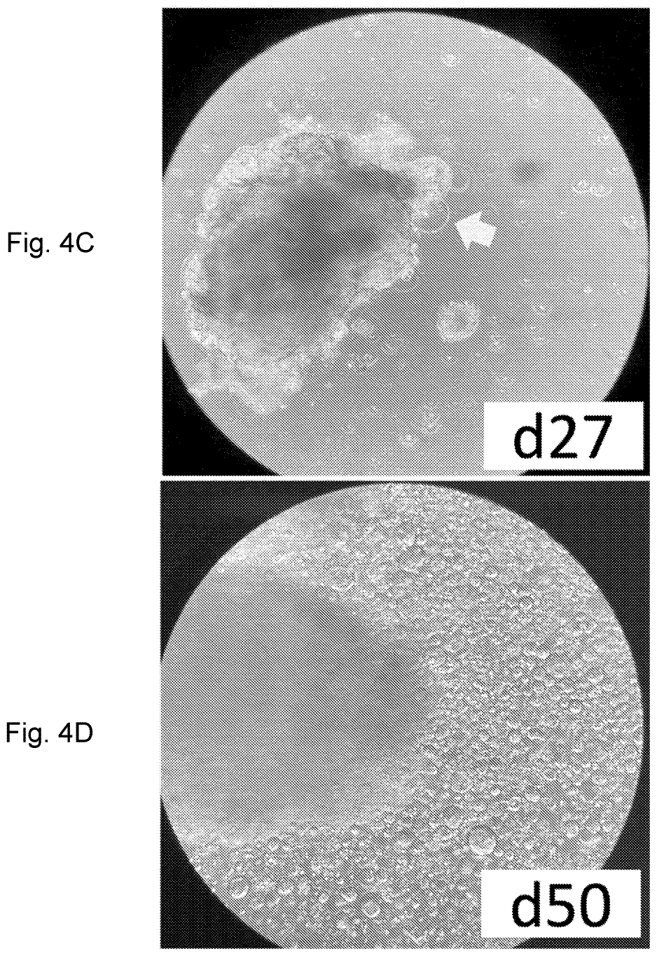

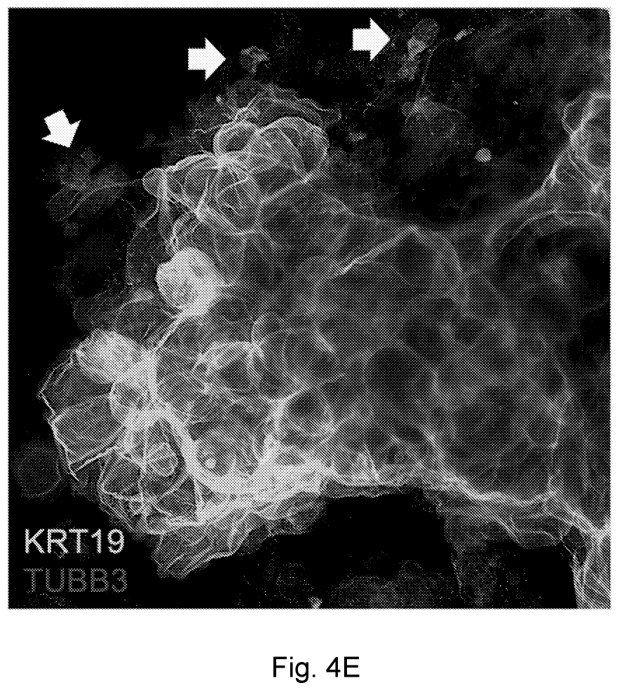

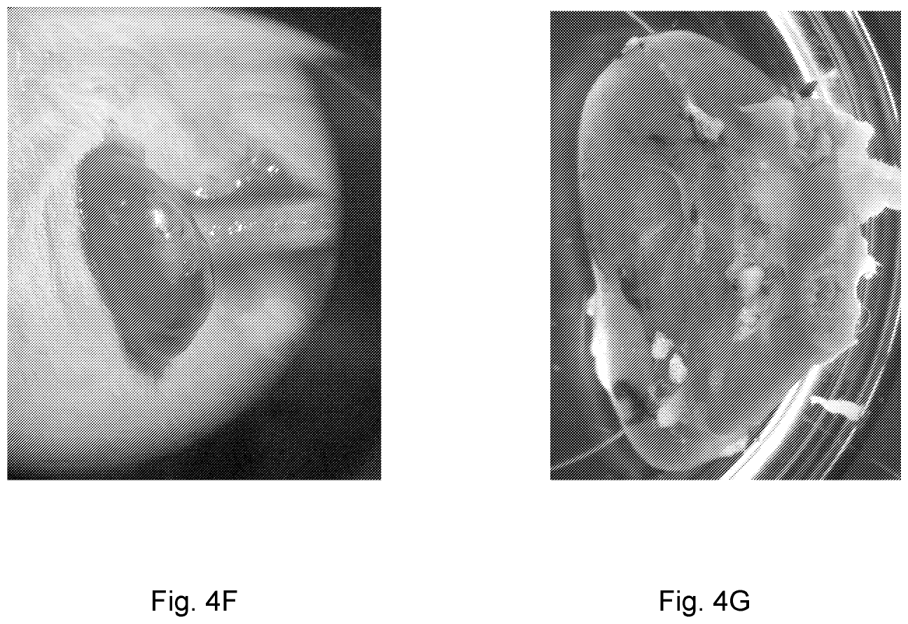

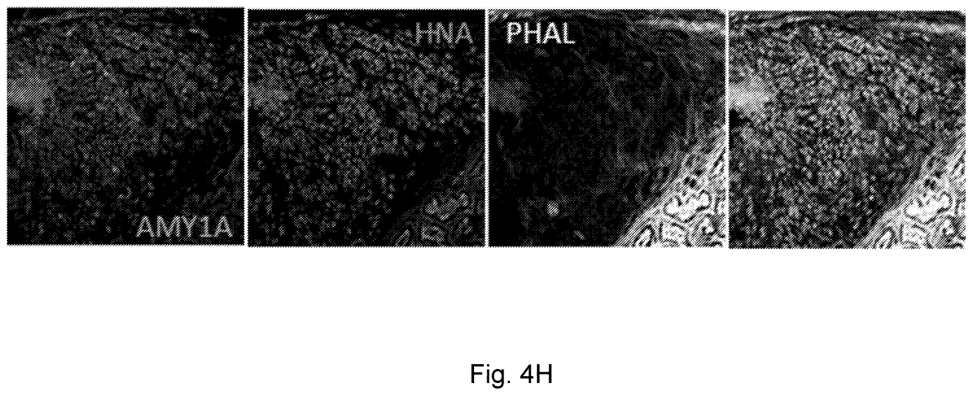

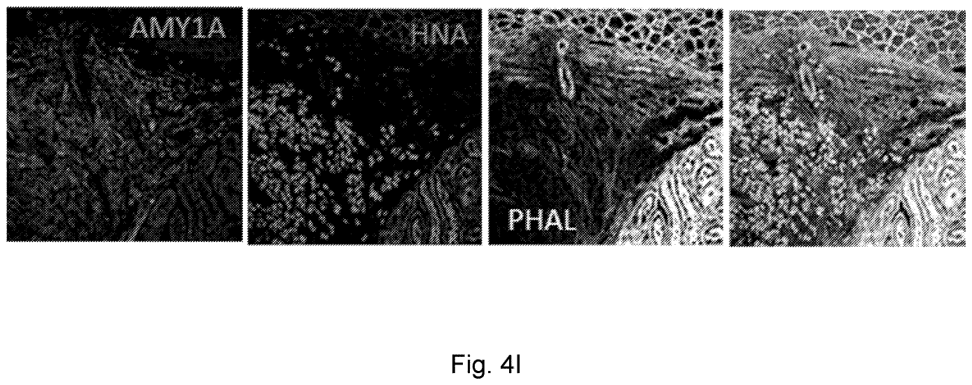

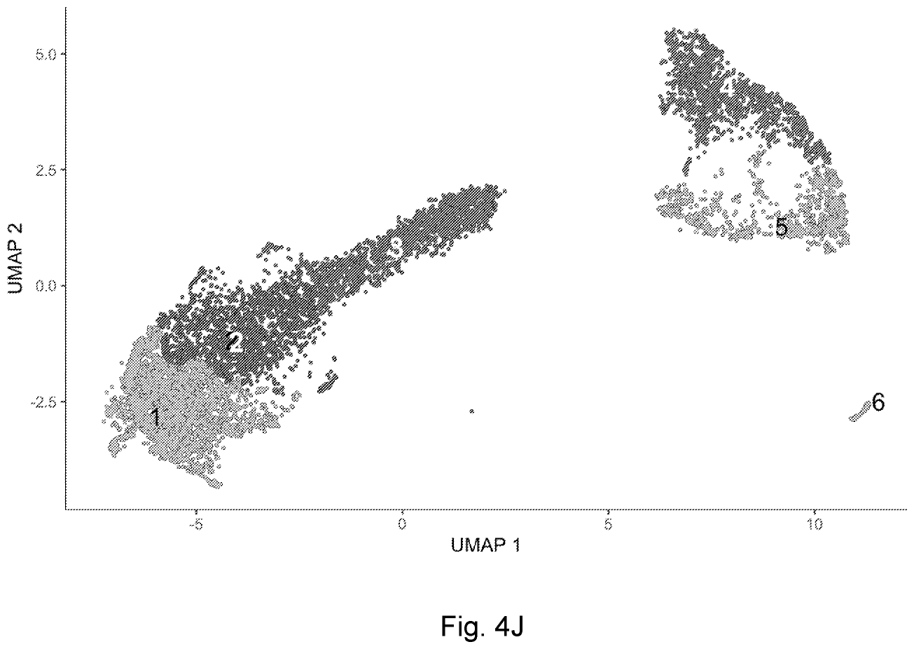

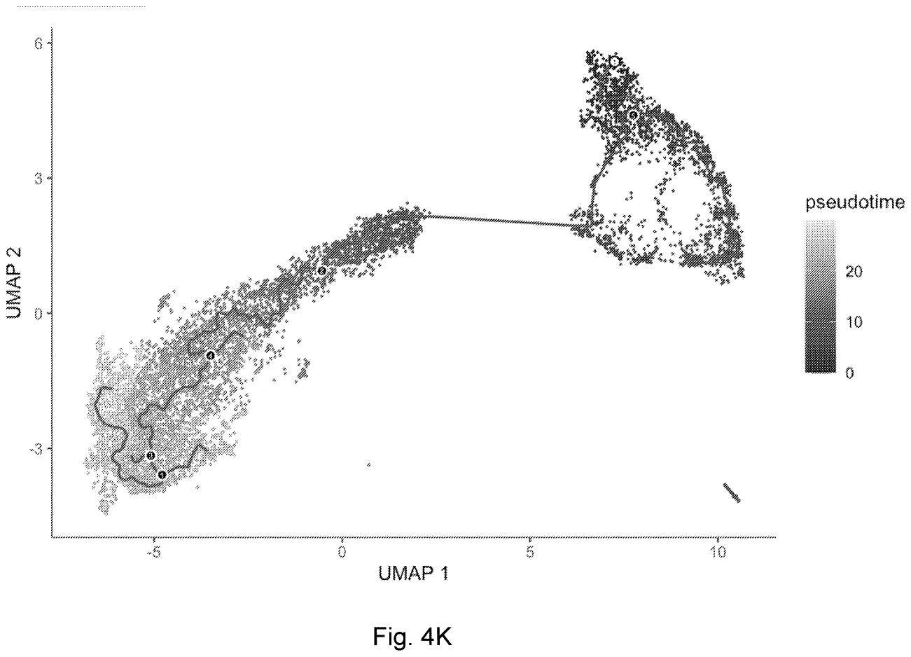

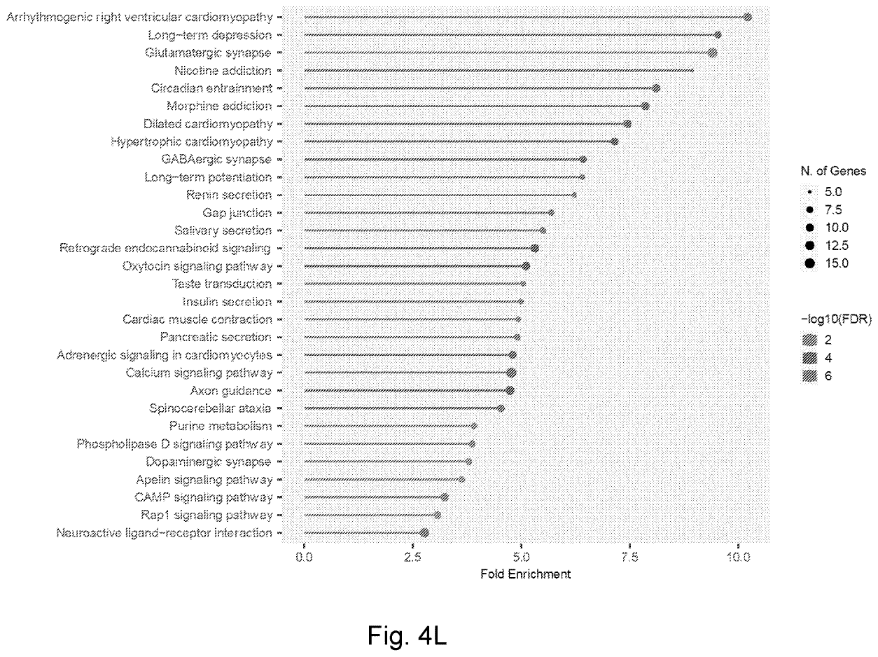

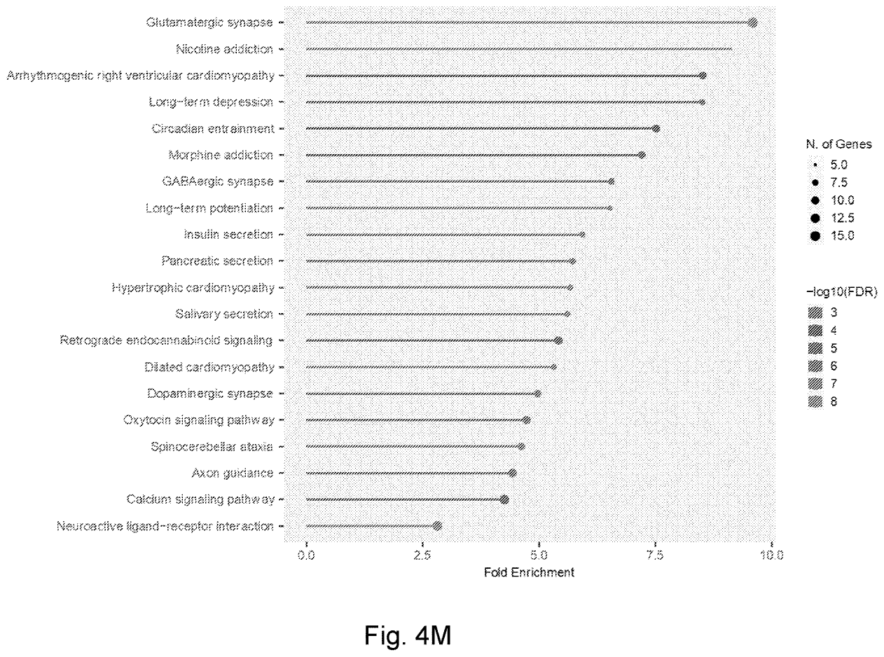

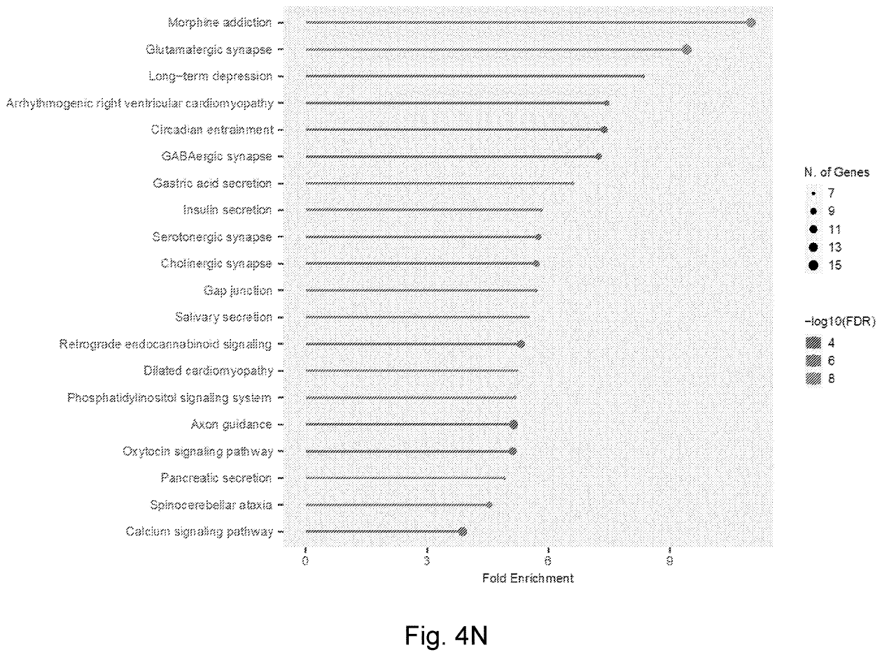

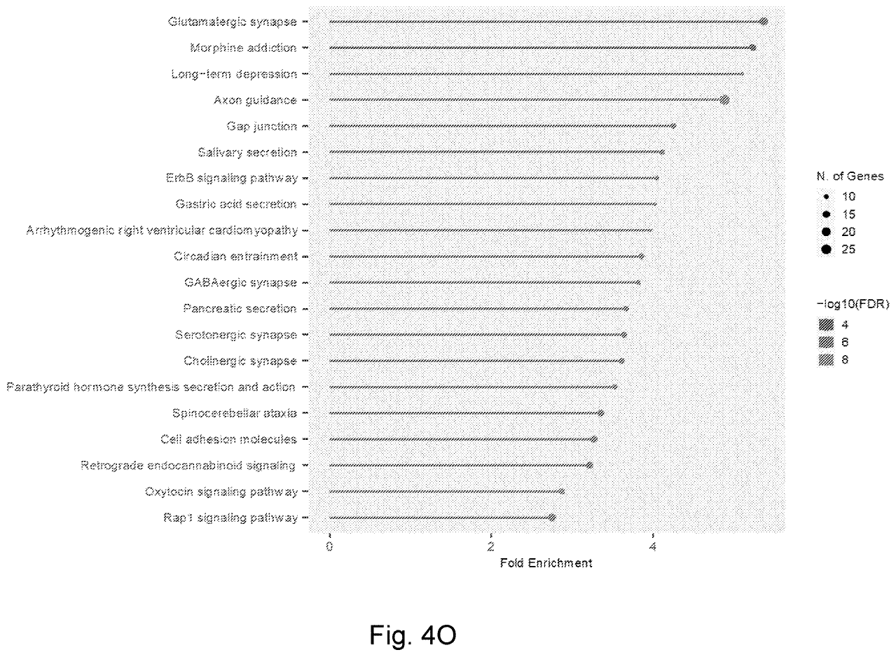

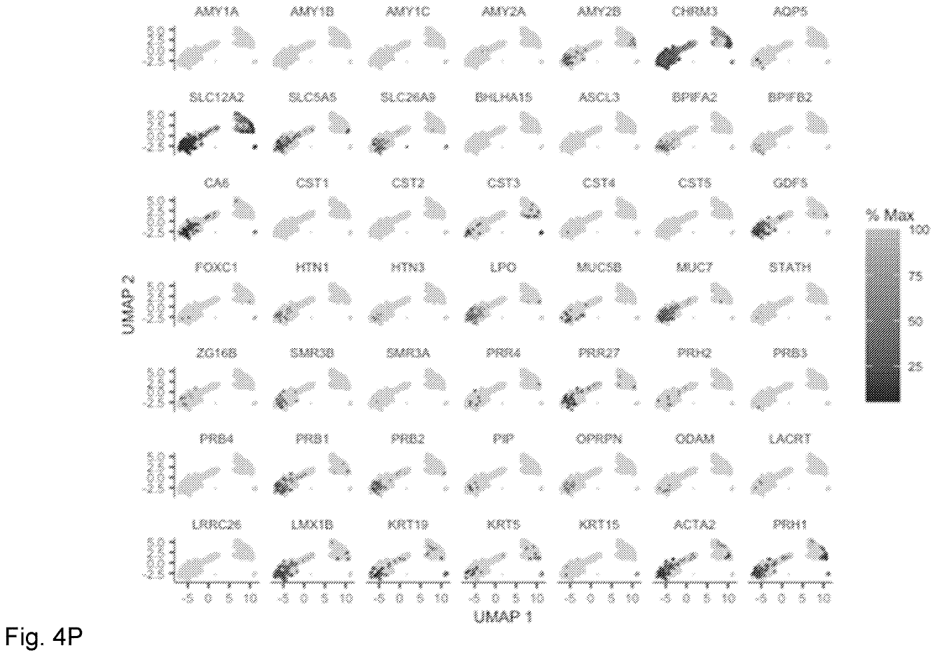

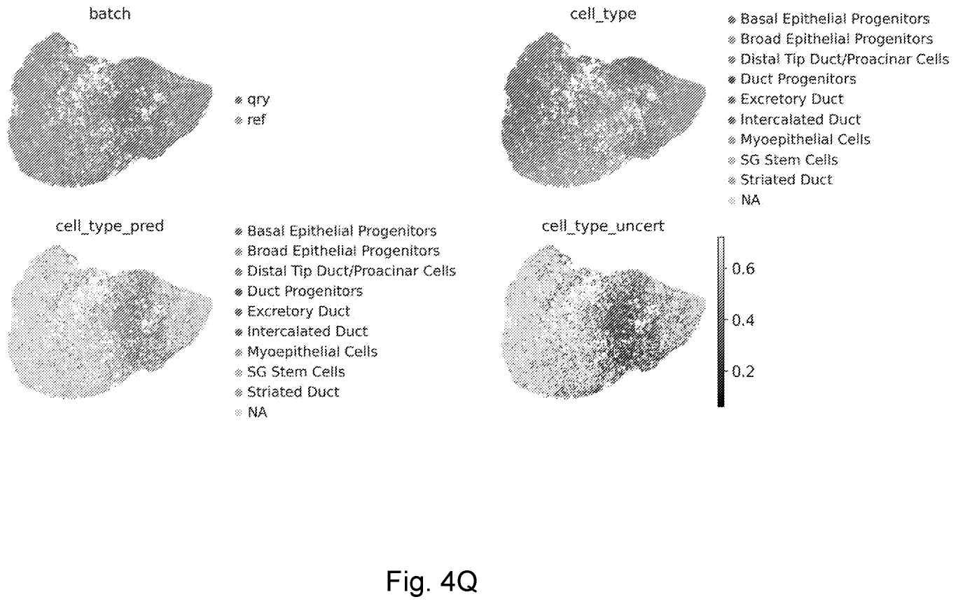

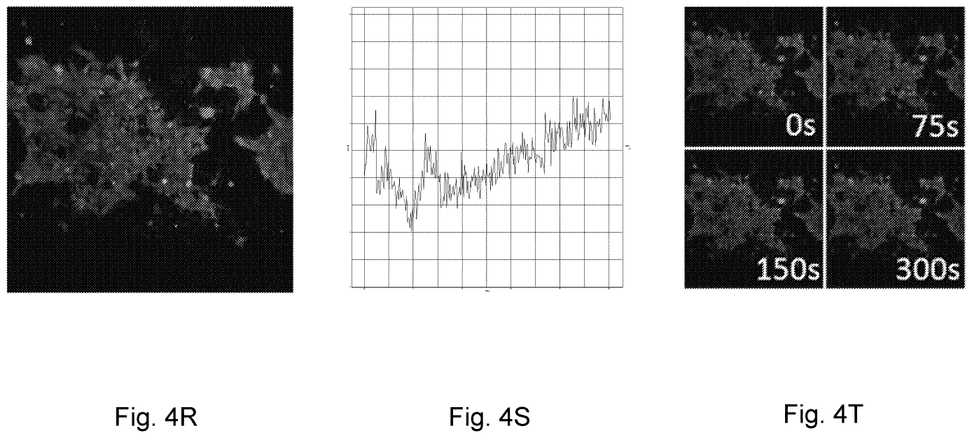

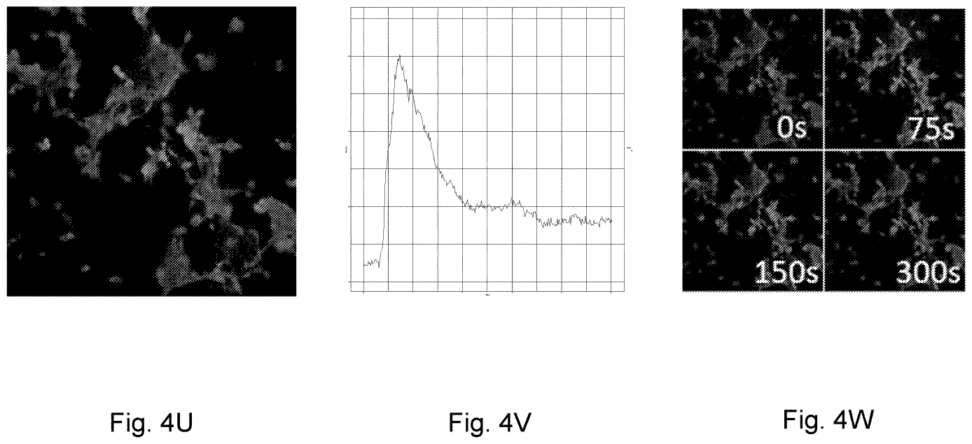

In some embodiments, the salivary gland organoid culture is implanted into the kidney capsule of a subject (see e.g., FIG. 4F-4G). Such implantation can be used to assess viability and growth in vivo. In some embodiments, the subject is an animal model, such as a mouse.

### Methods of Establishing Salivary Gland Organoid Cultures

In multiple aspects, described herein are methods of establishing a salivary gland organoid culture. In one aspect, the method comprises: (a) culturing a population of pluripotent stem cells in aggregation medium for a sufficient time to promote formation of pluripotent stem cell clusters; (b) culturing the resultant pluripotent stem cell clusters in a salivary epithelium differentiation medium for a sufficient time to promote differentiation into salivary epithelium; and (c) culturing the resultant salivary epithelium in a salivary gland organoid differentiation medium for a sufficient time to promote differentiation into a salivary gland organoid culture.

In some embodiments, the method comprises a step of culturing a population of pluripotent stem cells in aggregation medium for a sufficient time to promote formation of pluripotent stem cell clusters. Non-limiting examples of such aggregation media are described further herein.

In some embodiments, the method comprises a step of culturing the resultant pluripotent stem cell clusters in a salivary epithelium differentiation medium for a sufficient time to promote differentiation into salivary epithelium. Non-limiting examples of such salivary epithelium differentiation media are described further herein.

In some embodiments, the method comprises a step of culturing the resultant salivary epithelium in a salivary gland organoid differentiation medium for a sufficient time to promote differentiation into a salivary gland organoid culture. Non-limiting examples of such salivary gland organoid differentiation media are described further herein.

In some embodiments, a salivary gland organoid culture is maintained in a salivary gland organoid culture medium. Non-limiting examples of such salivary gland organoid culture media are described further herein.

In one aspect, the method comprises: (a) culturing pluripotent stem cell clusters in a salivary epithelium differentiation medium for a sufficient time to promote differentiation into salivary epithelium; and (b) culturing the resultant salivary epithelium in a salivary gland organoid differentiation medium for a sufficient time to promote differentiation into a salivary gland organoid culture.

In some embodiments, the pluripotent stem cells, pluripotent stem cell clusters, salivary epithelium, and/or salivary gland organoid culture are cultured within a 3-diminensal matrix, which can comprise a basement membrane matrix. A non-limiting example of such a basement membrane matrix is basement membrane matrix secreted by Engelbreth-Holm-Swann (EHS) mouse sarcoma cells (i.e., MATRIGEL®).

### Aggregation Media

In some embodiments, the method of establishing a salivary gland organoid culture comprises culturing a population of pluripotent stem cells in aggregation medium. In some embodiments, the population of pluripotent stem cells are cultured for a sufficient time to promote formation of pluripotent stem cell clusters.

In some embodiments, the pluripotent stem cells comprise induced pluripotent stem cells (iPSCs). In some embodiments, the pluripotent stem cells comprise mammalian induced pluripotent stem cells. In some embodiments, the pluripotent stem cells comprise human pluripotent stem cells (hiPSCs). In some embodiments, the iPSCs comprise cell line WTC-t 1 (CORIELL INSTITUTE, GM25256).

In some embodiments, the iPSCs are derived from a somatic cell sample from a subject. Non-limiting examples of a somatic cell include a fibroblast (e.g., a primary fibroblast), a muscle cell (e.g., a myocyte), a cumulus cell, a neural cell, a mammary cell, a hepatocyte and a pancreatic islet cell. In some embodiments, the somatic cell is a primary cell line or is the progeny of a primary or secondary cell line. In some embodiments, the somatic cell is obtained from a human sample, e.g. a hair follicle, a blood sample, a biopsy (e.g., a skin biopsy or an adipose biopsy), or a swab sample (e.g., an oral swab sample). Some further non-limiting examples of somatic cells include, but are not limited to. epithelial, endothelial, neuronal, adipose. cardiac, skeletal muscle, skin, immune cells, hepatic. splenic, lung, peripheral circulating blood cells, gastrointestinal, renal, bone marrow, and pancreatic cells. In some embodiments, a somatic cell can be a primary cell isolated from any somatic tissue including, but not limited to brain, liver, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In some embodiments, the somatic cell is a human somatic cell.

There are various methods for generating iPS cells. For example, any number of different somatic cell sources can be used to generate the iPS cells. Any somatic cell reprogramming approach known in the art can be used to generate the iPS cells used in the methods and compositions described herein. See e.g., Karami et al. “Induced pluripotent stem cells: Generation methods and new perspective in COVID-19 research, “Front Cell Dev Biol, 2022, 10: 1050856; Kumar et al. “Induced pluripotent stem cells: Mechanisms, achievements and perspectives in farm animals,” World J Stem Cells, 2015, 7(2): 315-328: Shi et al. “Induced pluripotent stem cell technology: a decade of progress,” Nature Reviews Drug Discovery 16, 115-130 (2017); the contents of each of which are incorporated herein by reference in their entireties.

In some embodiments, the iPS cell is derived from a subject with a disease or disorder that affects salivary gland function. Non-limiting examples of a disease or disorder that affects salivary gland function includes a metabolic disease, an inflammatory disease, an autoimmune disease, or radiation damage.

In some embodiments, the salivary gland organoid culture is cultured from induced pluripotent stem cells. In some embodiments, the salivary gland organoid culture is not cultured from non-pluripotent stem cells present in a tissue sample. The non-pluripotent stem cells can be tissue resident stem cells that are multipotent, oligopotent, or unipotent stem cells. In some embodiments, the salivary gland organoid culture is not cultured from non-pluripotent stem cells present in a salivary gland sample. In some embodiments, the salivary gland organoid culture is not cultured from non-pluripotent stem cells present in a pancreas sample. In some embodiments, the salivary gland organoid culture is not cultured from non-pluripotent stem cells present in a kidney sample. In some embodiments, the methods described herein do not comprise a step of dissecting a tissue sample (e.g., a sample from a salivary gland, pancreas, or kidney), such as to isolate or purify non-pluripotent stem cells from the tissue sample. In some embodiments, the methods described herein do not comprise a step of isolating or purifying pluripotent stem cells, pluripotent stem cell clusters, salivary epithelium, and/or salivary gland organoid cultures.

In some embodiments, the aggregation medium comprises an inhibitor of Rho Associated Coiled-Coil Containing Protein Kinase (ROCK). In some embodiments, the aggregation medium comprises an inhibitor of Rho Associated Coiled-Coil Containing Protein Kinase (ROCK) at a concentration of about 50 μM. In some embodiments, the inhibitor of ROCK is at a concentration of about 10 μM, about 20 μM, about 30 μM, about 40 μM, about 45 μM, about 50 μM. about 55 μM, about 60 μM, about 70 μM, about 80 μM about 90 μM, about 100 μM, about 45 μM to about 55 μM, about 40 μM to about 60 μM, about 35 μM to about 65 μM, about 30 μM to about 70 μM, about 25 μM to about 75 μM, about 20 μM to about 80 μM, about 15 μM to about 85 μM, about 10 μM to about 90 μM, about 5 μM to about 95 μM, or about 1 μM to about 100 μM. In some embodiments, the inhibitor of ROCK is at a concentration of at least 10 μM, at least 20 μM. at least 30 μM, at least 40 μM, at least 45 μM, at least 50 μM, at least 55 μM, at least 60 μM, at least 70 μM, at least 80 μM, at least 90 μM, or 100 μM. In some embodiments, the inhibitor of ROCK is at a concentration of at most 10 μM, at most 20 μM, at most 30 μM, at most 40 μM, at most 45 μM, at most 50 μM, at most 55 μM, at most 60 μM, at most 70 μM, at most 80 μM, at most 90 μM, or at most 100 μM. In some embodiments, the inhibitor of ROCK is Y-27632. In some embodiments, the inhibitor of ROCK is selected from the group consisting of AT-13148, BA-210, β-Elemene, Belumosudil. Chroman. DJ4, GSK-576371, GSK429286A (C21H16F4N402), H-1152, HA-1077 (Fasudil), Hydroxyfasudil (an active metabolite of fasudil), Ibuprofen. LX-7101. Netarsudil, RKI-1447. Ripasudil. TCS-7001, Thiazovivin, Verosudil (AR-12286), Y-27632, Y-30141, Y-33075. and Y-39983, or analogs or combinations thereof.

In some embodiments, the aggregation medium comprises mTeSR™1 medium. mTeSR™I medium is a complete, serum-free, defined formulation designed for the feeder-free maintenance and expansion of human embryonic stem (ES) cells or human induced pluripotent stem (iPS) cells in the undifferentiated state. In some embodiments, mTeSR™1 medium comprises DMEM/F12 (as described further herein), bovine serum albumin (BSA), basic fibroblast growth factor (bFGF), transforming growth factor beta (TGFβ), insulin, transferrin, cholesterol, lipids, pipecolic acid, gamma-aminobutyric acid (GABA), and/or β-mercaptoethanol.

In some embodiments, the sufficient amount of time to promote formation of pluripotent stem cell clusters is about 3 days. In some embodiments, the sufficient amount of time to promote formation of pluripotent stem cell clusters is about 1 day, about 2 days, about 3 days, about 4 days, about 5 days, about 6, or about 7 days. In some embodiments, the sufficient amount of time to promote formation of pluripotent stem cell clusters is at least 1 day, at least 2 days, at least 3 days, at least 4 days, at least 5 days, at least 6, or 7 days. In some embodiments, the sufficient amount of time to promote formation of pluripotent stem cell clusters is at most 1 day, at most 2 days, at most 3 days, at most 4 days, at most 5 days, at most 6, or at most 7 days. In some embodiments, the sufficient amount of time to promote formation of pluripotent stem cell clusters is 2-3 days, 3-4 days, 2-4 days, or 1-5 days.

In some embodiments, a pluripotent stem cell cluster is also referred to as a pluripotent stem cell aggregate or pluripotent stem cell colony. In some embodiments, a pluripotent stem cell cluster comprises about 5000 cells, about 6000 cells, about 7000 cells, about 8000 cells, about 9000 cells, about 10,000 cells, about 11,000 cells, about 12,000 cells, about 13.000 cells, about 14,000 cells, about 15,000 cells, about 16,000 cells, about 17,000 cells, about 18.000 cells, about 19,000 cells, about 20,000 cells, about 11,000-14,000 cells, about 10,000-15,000 cells, or more.

### Salivary Epithelium Differentiation Media

In some embodiments, the method of establishing a salivary gland organoid culture comprises culturing pluripotent stein cell clusters in a salivary epithelium differentiation medium. In some embodiments, the pluripotent stein cell clusters are cultured for a sufficient time to promote differentiation into salivary epithelium.

In some embodiments, salivary epithelium is characterized by the expression of salivary epithelium-specific markers, non-limiting examples of which are provided in Alghadeer et al., 2022, BioRxiv, available on the world wide web at biorxiv.org/content/10.101/2022.08.09.503399v1; Alghadeer et al., 2023. Dev Cell. 58(20): 2163-2180.e9; International Patent Publications WO2023129429A2, WO2024040050A2; the content of which is incorporated herein by reference in its entirety. For example, the salivary epithelium-specific marker can be Pituitary Homeobox 2 (PITX2).

In some embodiments, the salivary epithelium differentiation medium comprises: (a) an agonist of Smoothened; (b) a bone morphogenetic protein: (c) an inhibitor of a Glycogen Synthase Kinase (GSK) and/or an activator of a Wingless/Integrated (Wnt) and β-catenin pathway: (d) a growth factor for epithelium. (e) an inhibitor of a Bone Morphogenetic Protein type I (BMP I) receptor; (f) a neurotrophin; (g) a fibroblast growth factor; or any combinations thereof. Table 1 provides non-limiting examples of combinations of components in the salivary epithelium differentiation medium.

TABLE 1
Exemplary components of the salivary epithelium differentiation medium

| | | (c) GSK | | | | |
|---|---|---|---|---|---|---|
| | | inhibitor | | | | |
| | | and/or Wnt | | | | |
| | (b) bone | and β- | (d) growth | (e) BMP I | | (g) fibroblast |
| (a) agonist of | morphogenetic | catenin | factor for | receptor | | growth |
| Smoothened | protein | activator | epithelium | inhibitor | (f) neurotrophin | factor |
| X | | | | | | |
| | X | | | | | |
| X | X | | | | | |
| | | X | | | | |
| X | | X | | | | |
| | X | X | | | | |
| X | X | X | | | | |
| | | | X | | | |
| X | | | X | | | |
| | X | | X | | | |
| X | X | | X | | | |
| | | X | X | | | |
| X | | X | X | | | |
| | X | X | X | | | |
| X | X | X | X | | | |
| | | | | X | | |
| X | | | | X | | |
| | X | | | X | | |
| X | X | | | X | | |
| | | X | | X | | |
| X | | X | | X | | |
| | X | X | | X | | |
| X | X | X | | X | | |
| | | | X | X | | |
| X | | | X | X | | |
| | X | | X | X | | |
| X | X | | X | X | | |
| | | X | X | X | | |
| X | | X | X | X | | |
| | X | X | X | X | | |
| X | X | X | X | X | | |
| | | | | | X | |
| X | | | | | X | |
| | X | | | | X | |
| X | X | | | | X | |
| | | X | | | X | |
| X | | X | | | X | |
| | X | X | | | X | |
| X | X | X | | | X | |
| | | | X | | X | |
| X | | | X | | X | |
| | X | | X | | X | |
| X | X | | X | | X | |
| | | X | X | | X | |
| X | | X | X | | X | |
| | X | X | X | | X | |
| X | X | X | X | | X | |
| | | | | X | X | |
| X | | | | X | X | |
| | X | | | X | X | |
| × | X | | | X | X | |
| | | X | | X | X | |
| X | | X | | X | X | |
| | X | X | | X | X | |
| X | X | X | | X | X | |
| | | | X | X | X | |
| X | | | X | X | X | |
| | X | | X | X | X | |
| X | X | | X | X | X | |
| | | X | X | X | X | |
| X | | X | X | X | X | |
| | X | X | X | X | X | |
| X | X | X | X | X | X | |
| | | | | | | X |
| X | | | | | | X |
| | X | | | | | X |
| X | X | | | | | X |
| | | X | | | | X |
| X | | X | | | | X |
| | X | X | | | | X |
| X | X | X | | | | X |
| | | | X | | | X |
| X | | | X | | | X |
| | X | | X | | | X |
| X | X | | X | | | X |
| | | X | X | | | X |
| X | | X | X | | | X |
| | X | X | X | | | X |
| X | X | X | X | | | X |
| | | | | X | | X |
| X | | | | X | | X |
| | X | | | X | | X |
| X | X | | | X | | X |
| | | X | | X | | X |
| X | | X | | X | | X |
| | X | X | | X | | X |
| X | X | X | | X | | X |
| | | | X | X | | X |
| X | | | X | X | | X |
| | X | | X | X | | X |
| X | X | | X | X | | X |
| | | X | X | X | | X |
| X | | X | X | X | | X |
| | X | X | X | X | | X |
| X | X | X | X | X | | X |
| | | | | | X | X |
| X | | | | | X | X |
| | X | | | | X | X |
| X | X | | | | X | X |
| | | X | | | X | X |
| X | | X | | | X | X |
| | X | X | | | X | X |
| X | X | X | | | X | X |
| | | | X | | X | X |
| X | | | X | | X | X |
| | X | | X | | X | X |
| X | X | | X | | X | X |
| | | X | X | | X | X |
| X | | X | X | | X | X |
| | X | X | X | | X | X |
| X | X | X | X | | X | X |
| | | | | X | X | X |
| X | | | | X | X | X |
| | X | | | X | X | X |
| X | X | | | X | X | X |
| | | X | | X | X | X |
| X | | X | | X | X | X |
| | X | X | | X | X | X |
| X | X | X | | X | X | X |
| | | | X | X | X | X |
| X | | | X | X | X | X |
| | X | | X | X | X | X |
| X | X | | X | X | X | X |
| | | X | X | X | X | X |
| X | | X | X | X | X | X |
| | X | X | X | X | X | X |
| X | X | X | X | X | X | X |

In some embodiments, the salivary epithelium differentiation medium comprises an agonist of Smoothened from about day 0 to about day 8 after beginning the salivary epithelium differentiation medium. In some embodiments, the salivary epithelium differentiation medium comprises an agonist of Smoothened on day 0. day 1, day 2, day 3, day 4, day 5, day 6. day 7, day 8, or any combination thereof, after beginning the salivary epithelium differentiation medium. In some embodiments, the salivary epithelium differentiation medium comprises an agonist of Sinoothened at a concentration of about 400 nM. In some embodiments, the salivary epithelium differentiation medium comprises an agonist of Smoothened at a concentration of about 1 mM. In some embodiments, the salivary epithelium differentiation medium comprises an agonist of Sinoothened at a concentration of about 100 nM, about 200 nM, about 300 nM, about 350 nM, about 400 nM, about 450 nM, about 500 nM, about 600 nM, about 700 nM, about 800 nM, about 900 nM, about 1 μM, about 10 μM, about 200 μM, about 100 μM, about 300 μM, about 400 μM, about 500 μM, about 600 μM, about 700 μM, about 800 μM, about 900 μM, about 1 mM, about 2 mM, about 3 mM, about 4 mM, about 5 mM, about 6 mM, about 7 mM, about 8 mM, about 9 mM, about 10 mM, 100-500 nM, 200-600 nM, 350-450 nM, 0.9 mM-1.1 mM. 0.75-1.25 mM, or 0.5-1.5 mM. In some embodiments, the salivary epithelium differentiation medium comprises an agonist of Smoothened at a concentration of at least 100 nM, at least 200 nM, at least 300 nM, at least 350 nM, at least 400 nM, at least 450 nM, at least 500 nM, at least 600 nM, at least 700 nM, at least 800 nM, at least 900 nM, at least 1 μM, at least 10 μM, at least 200 μM, at least 100 μM, at least 300 μM, at least 400 μM, at least 500 μM, at least 600 μM, at least 700 μM, at least 800 μM, at least 900 μM, at least 1 mM, at least 2 mM, at least 3 mM, at least 4 mM, at least 5 mM, at least 6 mM, at least 7 mM, at least 8 mM, at least 9 mM, or 10 mM. In some embodiments, the salivary epithelium differentiation medium comprises an agonist of Smoothened at a concentration of at most 100 nM, at most 200 nM, at most 300 nM, at most 350 nM, at most 400 nM, at most 450 nM, at most 500 nM, at most 600 nM, at most 700 nM, at most 800 nM, at most 900 nM, at most 1 μM, at most 10 μM, at most 200 μM, at most 100 μM, at most 300 μM, at most 400 μM, at most 500 μM, at most 600 μM, at most 700 μM, at most 800 μM, at most 900 μM, at most 1 mM, at most 2 mM, at most 3 mM, at most 4 mM, at most 5 mM, at most 6 mM, at most 7 mM, at most 8 mM, at most 9 mM, or at most 10 mM. In some embodiments, the agonist of Smoothened is Smoothened Agonist (SAG). In some embodiments, the agonist of Smoothened is Smoothened agonist (SAG), purmorphamine, or an oxysterol (e.g., 20(S)-OHC and 20(S)-yne).

In some embodiments, the salivary epithelium differentiation medium comprises a bone morphogenetic protein from about day 3 to about day 8 after beginning the salivary epithelium differentiation medium. In some embodiments, the salivary epithelium differentiation medium comprises a bone morphogenetic protein on day 3, day 4, day 5, day 6, day 7, day 8, or any combination thereof, after beginning the salivary epithelium differentiation medium. In some embodiments, the salivary epithelium differentiation medium comprises a bone morphogenetic protein at a concentration of about 150 pM. In some embodiments, the salivary epithelium differentiation medium comprises a bone morphogenetic protein at a concentration of about 10 pM about 50 pM, about 100 pM, about 110 pM, about 120 pM, about 130 pM, about 140 pM, about 150 pM, about 160 pM, about 170 pM, about 180 pM, about 190 pM. about 200 pM, about 300 pM, about 400 pM, about 500 pM, 125-175 pM, 100-200 pM, or 50-250 pM. In some embodiments, the salivary epithelium differentiation medium comprises a bone morphogenetic protein at a concentration of at least 10 pM at least 50 pM, at least 100 pM, at least 110 pM, at least 120 pM, at least 130 pM, at least 140 pM, at least 150 pM, at least 160 pM, at least 170 pM, at least 180 pM, at least 190 pM, at least 200 pM, at least 300 pM, at least 400 pM, or 500 pM. In some embodiments, the salivary epithelium differentiation medium comprises a bone morphogenetic protein at a concentration of at most 10 pM at most 50 pM, at most 100 pM, at most 110 pM, at most 120 pM, at most 130 pM, at most 140 pM, at most 150 pM, at most 160 pM, at most 170 pM, at most 180 pM, at most 190 pM, at most 200 pM, at most 300 pM, at most 400 pM, or at most 500 pM. In some embodiments, the bone morphogenetic protein is bone morphogenetic protein 4 (BMP4). In some embodiments, the bone morphogenetic protein is mammalian bone morphogenetic protein 4 (BMP4). In some embodiments, the bone morphogenetic protein is human bone morphogenetic protein 4 (BMP4). In some embodiments, the bone morphogenetic protein (BMP) is selected from the group consisting of BMP1, BMP2, BMP3, BMP4, BMP5, BMP6, BMP7, BMP8A, BMP8B, BMP10, and BMP15, or combinations thereof.

In some embodiments, the salivary epithelium differentiation medium comprises an agonist of Smoothened, an inhibitor of a Glycogen Synthase Kinase (GSK), an activator of a Wingless/Integrated (Wnt) and D-catenin pathway, a growth factor for epithelium, a neurotrophin, or any combination thereof (see e.g., Table 1) from about day 8 to about day 12 after beginning the salivary epithelium differentiation medium, or on day 8, day 9, day 10, day 11, day 12, or any combination thereof after beginning the salivary epithelium differentiation medium.

In some embodiments, the salivary epithelium differentiation medium comprises an inhibitor of a Glycogen Synthase Kinase (GSK) and/or activator of a Wingless/Integrated (Wnt) and β-catenin pathway at a concentration of about 5 μM. In some embodiments, the salivary epithelium differentiation medium comprises an inhibitor of a Glycogen Synthase Kinase (GSK) and/or activator of a Wingless/Integrated (Wnt) and β-catenin pathway at a concentration of about 0.5 μM, about 1 μM, about 2 μM, about 3 μM, about 4 μM, about 5 μM, about 6 μM, about 7 μM, about 8 μM, about 9 μM, about 10 μM, 4.5-5.5 μM, 4-6 μM, 3-7 μM, 2-8 μM, 1-9 μM, or 0.5-10 μM. In some embodiments, the salivary epithelium differentiation medium comprises an inhibitor of a Glycogen Synthase Kinase (GSK) and/or activator of a Wingless/Integrated (Wnt) and D-catenin pathway at a concentration of at least 0.5 μM, at least 1 μM, at least 2 μM, at least 3 μM, at least 4 μM, at least 5 μM, at least 6 μM, at least 7 μM, at least 8 μM, at least 9 μM, or 10 μM. In some embodiments, the salivary epithelium differentiation medium comprises an inhibitor of a Glycogen Synthase Kinase (GSK) and/or activator of a Wingless/Integrated (Wnt) and β-catenin pathway at a concentration of at most 0.5 μM, at most 1 μM, at most 2 μM, at most 3 μM, at most 4 μM, at most 5 μM, at most 6 μM, at most 7 μM, at most 8 μM, at most 9 μM, or at most 10 μM. In some embodiments, the inhibitor of GSK and/or activator of the Wnt and β-catenin pathway is Chiron (Chir99021). In some embodiments, the inhibitor of GSK and/or activator of the Wnt and β-catenin pathway is selected from the group consisting of Chir99021, cromolyn sodium, lithium chloride, azakenpaullone (1-Akp), tideglusib, 6-bromoindirubin-3, Wnt-1, Wnt-2, Wnt-2b, Wnt-3a, Wnt-4, Wnt-5a, Wnt-5b, Wnt-6, Wnt-7a, Wnt-7a/b, R-spondin (RSPO), and Norrin, or analogs or combinations thereof.

In some embodiments, the salivary epithelium differentiation medium comprises a growth factor for epithelium at a concentration of about 15 ng/mL. In some embodiments, the salivary epithelium differentiation medium comprises a growth factor for epithelium at a concentration of about 1 ng/mL, about 2 ng/mL, about 3 ng/mL, about 4 ng/mL, about 5 ng/nL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 25 ng/mL, about 30 ng/mL, 14.5-15.5 ng/mL, 14-16 ng/mL, 13-17 ng/mL, 12-18 ng/mL, 11-19 ng/mL, 10-20 ng/mL, 5-25 ng/mL, or 1-30 ng/mL. In some embodiments, the salivary epithelium differentiation medium comprises a growth factor for epithelium at a concentration of at least 1 ng/mL, at least 2 ng/mL, at least 3 ng/mL, at least 4 ng/mL, at least 5 ng/mL, at least 6 ng/mL, at least 7 ng/mL, at least 8 ng/mL, at least 9 ng/mL, at least 10 ng/mL, at least 11 ng/mL, at least 12 ng/mL, at least 13 ng/mL, at least 14 ng/mL, at least 15 ng/mL, at least 16 ng/mL, at least 17 ng/mL, at least 18 ng/mL, at least 19 ng/mL, at least 20 ng/mL, at least 25 ng/mL, or 30 ng/mL. In some embodiments, the salivary epithelium differentiation medium comprises a growth factor for epithelium at a concentration of at most 1 ng/mL, at most 2 ng/mL, at most 3 ng/mL, at most 4 ng/mL, at most 5 ng/mL, at most 6 ng/mL, at most 7 ng/mL, at most 8 ng/mL, at most 9 ng/mL, at most 10 ng/mL, at most 11 ng/mL, at most 12 ng/mL, at most 13 ng/mL, at most 14 ng/mL, at most 15 ng/mL, at most 16 ng/mL, at most 17 ng/mL, at most 18 ng/mL, at most 19 ng/mL, at most 20 ng/mL, at most 25 ng/mL, or at most 30 ng/mL. In some embodiments, the growth factor for epithelium is epidermal growth factor (EGF), which can also be referred to interchangeably as epithelial growth factor. In some embodiments, the growth factor for epithelium is mammalian epidermal growth factor (EGF). In some embodiments, the growth factor for epithelium is human epidermal growth factor (EGF). In some embodiments, the growth factor for epithelium is selected from the group consisting of Heparin Binding EGF Like Growth Factor (HB-EGF), Transforming Growth Factor Alpha (TGF-α), epigen, neuregulin (e.g., NRG1, NRG2, NRG3, NRG4), amphiregulin, an EGF-Like Protein (e.g., EGFL6, EGFL7, EGFL8), betacellulin, and a tomoregulin (e.g., TMEFF1, TMEFF2).

In some embodiments, the salivary epithelium differentiation medium comprises an inhibitor of a Bone Morphogenetic Protein type I (BMP I) receptor at a concentration of about 1 μM. In some embodiments, the salivary epithelium differentiation medium comprises an inhibitor of a Bone Morphogenetic Protein type I (BMP I) receptor at a concentration of about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 2 μM, about 3 μM, about 4 μM, about 5 μM, about 6 μM, about 7 μM, about 8 μM, about 9 μM, about 10 μM, 0.9-1.1 μM, 0.75-1.25 μM, 0.5-1.5 μM, 0.1-2 μM, 0.1-5 μM, or 0.1-10 μM. In some embodiments, the salivary epithelium differentiation medium comprises an inhibitor of a Bone Morphogenetic Protein type I (BMP I) receptor at a concentration of at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, at least 1 μM, at least 2 μM, at least 3 μM, at least 4 μM, at least 5 μM, at least 6 μM, at least 7 μM, at least 8 μM, at least 9 μM, or 10 μM. In some embodiments, the salivary epithelium differentiation medium comprises an inhibitor of a Bone Morphogenetic Protein type I (BMP I) receptor at a concentration of at most 0.1 μM, at most 0.2 μM, at most 0.3 μM, at most 0.4 μM, at most 0.5 μM, at most 0.6 μM, at most 0.7 μM, at most 0.8 μM, at most 0.9 μM, at most 1 μM, at most 2 μM, at most 3 μM, at most 4 μM, at most 5 μM, at most 6 μM, at most 7 μM, at most 8 μM, at most 9 μM, or at most 10 μM. In some embodiments, the inhibitor of the BMP type I receptor is LDN-193189. In some embodiments, the inhibitor of the BMP type I receptor is selected from the group consisting of Dorsomorphin, LDN-193189, LDN-214117, LDN-212854, SB-431452, DMH1, and K02288a, or analogs or combinations thereof: see e.g.. Dinter et al. Methods Mol Biol. 2019:1891:221-233. the contents of which are incorporated herein by reference in their entirety.

In some embodiments, the salivary epithelium differentiation medium comprises a neurotrophin at a concentration of about 1 μM. In some embodiments, the salivary epithelium differentiation medium comprises a neurotrophin at a concentration of about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 2 μM, about 3 μM, about 4 μM, about 5 μM, about 6 μM, about 7 μM, about 8 μM, about 9 μM, about 10 μM, 0.9-1.1 μM, 0.75-1.25 μM, 0.5-1.5 μM, 0.1-2 μM, 0.1-5 μM, or 0.1-10 μM. In some embodiments, the salivary epithelium differentiation medium comprises a neurotrophin at a concentration of at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, at least 1 μM, at least 2 μM, at least 3 μM, at least 4 μM, at least 5 μM, at least 6 μM, at least 7 μM, at least 8 μM, at least 9 μM, or 10 μM. In some embodiments, the salivary epithelium differentiation medium comprises a neurotrophin at a concentration of at most 0.1 μM, at most 0.2 μM, at most 0.3 μM, at most 0.4 μM, at most 0.5 μM, at most 0.6 μM, at most 0.7 μM, at most 0.8 μM, at most 0.9 μM, at most 1 μM, at most 2 μM, at most 3 μM, at most 4 μM, at most 5 μM, at most 6 μM, at most 7 μM, at most 8 μM, at most 9 μM, or at most 10 μM. In some embodiments, the neurotrophin is Neurotrophin-4. In some embodiments, the neurotrophin is mammalian Neurotrophin-4. In some embodiments, the neurotrophin is human Neurotrophin-4. In some embodiments, the neurotrophin is Nerve growth factor (NGF), Brain-derived neurotrophic factor (BDNF), Neurotrophin 3 (NT-3), Neurotrophin 4 (NT-4), or Neurotrophin 5 (NT-5).

In some embodiments, the salivary epithelium differentiation medium comprises a fibroblast growth factor from about day 10 to about day 12 after beginning the salivary epithelium differentiation medium. In some embodiments, the salivary epithelium differentiation medium comprises a fibroblast growth factor on day 10, day 11, day 12, day 10-11, or day 11-12 after beginning the salivary epithelium differentiation medium. In some embodiments, the salivary epithelium differentiation medium comprises a fibroblast growth factor at a concentration of about 250 ng/mL. In some embodiments, the salivary epithelium differentiation medium comprises a fibroblast growth factor at a concentration of about 50 ng/mL, about 100 ng/mL, about 150 ng/mL, about 200 ng/mL, about 210 ng/mL, about 220 ng/mL, about 230 ng/mL, about 240 ng/mL, about 250 ng/mL, about 260 ng/mL, about 270 ng/mL, about 280 ng/mL, about 290 ng/mL, about 300 ng/mL, about 400 ng/mL, about 500 ng/mL, 240-260 ng/mL, 225-275 ng/mL, 200-300 ng/mL, 100-500 ng/mL, or 50-500 ng/mL. In some embodiments, the salivary epithelium differentiation medium comprises a fibroblast growth factor at a concentration of at least 50 ng/mL, at least 100 ng/mL, at least 150 ng/mL, at least 200 ng/mL, at least 210 ng/mL, at least 220 ng/mL, at least 230 ng/mL, at least 240 ng/mL, at least 250 ng/mL, at least 260 ng/mL, at least 270 ng/mL, at least 280 ng/mL, at least 290 ng/mL, at least 300 ng/mL, at least 400 ng/mL, or 500 ng/mL. In some embodiments, the salivary epithelium differentiation medium comprises a fibroblast growth factor at a concentration of at most 50 ng/mL, at most 100 ng/mL, at most 150 ng/mL, at most 200 ng/mL, at most 210 ng/mL, at most 220 ng/mL, at most 230 ng/mL, at most 240 ng/mL, at most 250 ng/mL, at most 260 ng/mL, at most 270 ng/mL, at most 280 ng/mL, at most 290 ng/mL, at most 300 ng/mL, at most 400 ng/mL, or at most 500 ng/mL. In some embodiments, the fibroblast growth factor is fibroblast growth factor 10 (FGF10). In some embodiments, the fibroblast growth factor is mammalian fibroblast growth factor 10 (FGF10). In some embodiments, the fibroblast growth factor is human fibroblast growth factor 10 (FGF10). In some embodiments, the fibroblast growth factor is fibroblast growth factor 10 (FGF10), fibroblast growth factor 7 (FGF7), and/or fibroblast growth factor 1 (FGF1). In some embodiments, the fibroblast growth factor (FGF) is selected from the group consisting of FGF1, FGF2, FGF3, FGF4, FGF5, FGF6, FGF7, FGF8, FGF9, FGF10, FGF16, FGF17, FGF18, FGF19, FGF20, FGF21, FGF22, and FGF23, or any combination thereof.

In some embodiments, the salivary epithelium differentiation medium comprises EPICULT™-C medium. EPICULT™-C Medium is a serum-free culture medium optimized for the short-term culture of mammary luminal or myoepithelial cells, e.g., human mammary luminal or human myoepithelial cells.

In some embodiments, the sufficient amount of time to promote differentiation of pluripotent stem cell clusters into salivary epithelium is about 12 days. In some embodiments, the sufficient amount of time to promote differentiation of pluripotent stem cell clusters into salivary epithelium is about 10 days, about 11 days, about 12 days, about 13 days, about 14 days, about 15 days, about 16, or about 17 days. In some embodiments, the sufficient amount of time to promote differentiation of pluripotent stem cell clusters into salivary epithelium is at least 10 days, at least 11 days, at least 12 days, at least 13 days, at least 14 days, at least 15 days. at least 16, or 17 days. In some embodiments, the sufficient amount of time to promote differentiation of pluripotent stem cell clusters into salivary epithelium is at most 10 days, at most 11 days, at most 12 days, at most 13 days, at most 14 days, at most 15 days, at most 16, or at most 17 days. In some embodiments, the sufficient amount of time to promote differentiation of pluripotent stem cell clusters into salivary epithelium is 11-12 days, 12-13 days, 11-13 days, 10-14 days, or 10-15 days.

### Salivary Gland Organoid Differentiation Media

In some embodiments, the method of establishing a salivary gland organoid culture comprises culturing salivary epithelium in a salivary gland organoid differentiation medium. In some embodiments, the salivary epithelium is cultured for a sufficient time to promote differentiation into a salivary gland organoid culture.

In some embodiments, the salivary gland organoid differentiation medium comprises: (a) a medium supplement; (b) a fibroblast growth factor: (c) a neuregulin; (d) an extracellular matrix glycoprotein and/or a basement membrane matrix: (e) an inhibitor of Rho Associated Coiled-Coil Containing Protein Kinase (ROCK); (f) a growth factor for epithelium; (g) an inhibitor of a Glycogen Synthase Kinase (GSK) and/or activator of a Wingless/Integrated (Wnt) and β-catenin pathway; (h) an inhibitor of transforming growth factor-β (TGF-β) type I receptor; or any combinations thereof.

In some embodiments, the salivary gland organoid differentiation medium comprises: (a) a medium supplement and at least one of the following: (b) a fibroblast growth factor; (c) a neuregulin; (d) an extracellular matrix glycoprotein and/or a basement membrane matrix; (e) an inhibitor of Rho Associated Coiled-Coil Containing Protein Kinase (ROCK); (f) a growth factor for epithelium; (g) an inhibitor of a Glycogen Synthase Kinase (GSK) and/or activator of a Wingless/Integrated (Wnt) and β-catenin pathway; (h) an inhibitor of transforming growth factor-β (TGF-β) type I receptor; or any combinations thereof. Table 2 provides non-limiting examples of combinations of components in the salivary gland organoid differentiation medium.

TABLE 2
Exemplary components of the salivary gland organoid differentiation medium (each can be
in combination with (a) a medium supplement as described herein, see e.g., Table 3)

| | | (d) extracellular | | | | |
|---|---|---|---|---|---|---|
| | | matrix | | | (g) GSK | |
| | | glycoprotein | | | inhibitor | |
| | | and/or | | | and/or Wnt | (h) TGF-β |
| (b) fibroblast | | basement | | (f) growth | and β- | type I |
| growth | | membrane | (e) ROCK | factor for | catenin | receptor |
| factor | (c) neuregulin | matrix | inhibitor | epithelium | activator | inhibitor |
| X | | | | | | |
| | X | | | | | |
| X | X | | | | | |
| | | X | | | | |
| X | | X | | | | |
| | X | X | | | | |
| X | X | X | | | | |
| | | | X | | | |
| X | | | X | | | |
| | X | | X | | | |
| X | X | | X | | | |
| | | X | X | | | |
| X | | X | X | | | |
| | X | X | X | | | |
| X | X | X | X | | | |
| | | | | X | | |
| X | | | | X | | |
| | X | | | X | | |
| X | X | | | X | | |
| | | X | | X | | |
| X | | X | | X | | |
| | X | X | | X | | |
| X | X | X | | X | | |
| | | | X | X | | |
| X | | | X | X | | |
| | X | | X | X | | |
| X | X | | X | X | | |
| | | X | X | X | | |
| X | | X | X | X | | |
| | X | X | X | X | | |
| X | X | X | X | X | | |
| | | | | | X | |
| X | | | | | X | |
| | X | | | | X | |
| X | X | | | | X | |
| | | X | | | X | |
| X | | X | | | X | |
| | X | X | | | X | |
| X | X | X | | | X | |
| | | | X | | X | |
| X | | | X | | X | |
| | X | | X | | X | |
| | X | | X | | X | |
| | | X | X | | X | |
| X | | X | X | | X | |
| | X | X | X | | X | |
| X | X | X | X | | X | |
| | | | | X | X | |
| X | | | | X | X | |
| | X | | | X | X | |
| X | X | | | X | X | |
| | | X | | X | X | |
| X | | X | | X | X | |
| | X | X | | X | X | |
| X | X | X | | X | X | |
| | | | X | X | X | |
| X | | | X | X | X | |
| | X | | X | X | X | |
| X | X | | X | X | X | |
| | | X | X | X | X | |
| X | | X | X | X | X | |
| | X | X | X | X | X | |
| X | X | X | X | X | X | |
| | | | | | | X |
| X | | | | | | X |
| | X | | | | | X |
| X | X | | | | | X |
| | | X | | | | X |
| X | | X | | | | X |
| | X | X | | | | X |
| X | X | X | | | | X |
| | | | X | | | X |
| X | | | X | | | X |
| | X | | X | | | X |
| X | X | | X | | | X |
| | | X | X | | | X |
| X | | X | X | | | X |
| | X | X | X | | | X |
| X | X | X | X | | | X |
| | | | | X | | X |
| X | | | | X | | X |
| | X | | | X | | X |
| X | X | | | X | | X |
| | | X | | X | | X |
| X | | X | | X | | X |
| | X | X | | X | | X |
| X | X | X | | X | | X |
| | | | X | X | | X |
| X | | | X | X | | X |
| | X | | X | X | | X |
| X | X | | X | X | | X |
| | | X | X | X | | X |
| X | | X | X | X | | X |
| | X | X | X | X | | X |
| X | X | X | X | X | | X |
| | | | | | X | X |
| X | | | | | X | X |
| | X | | | | X | X |
| X | X | | | | X | X |
| | | X | | | X | X |
| X | | X | | | X | X |
| | X | X | | | X | X |
| X | X | X | | | X | X |
| | | | X | | X | X |
| X | | | X | | X | X |
| | X | | X | | X | X |
| X | X | | X | | X | X |
| | | X | X | | X | X |
| X | | X | X | | X | X |
| | X | X | X | | X | X |
| X | X | X | X | | X | X |
| | | | | X | X | X |
| X | | | | X | X | X |
| | X | | | X | X | X |
| X | X | | | X | X | X |
| | | X | | X | X | X |
| X | | X | | X | X | X |
| | X | X | | X | X | X |
| X | X | X | | X | X | X |
| | | | X | X | X | X |
| X | | | X | X | X | X |
| | X | | X | X | X | X |
| X | X | | X | X | X | X |
| | | X | X | X | X | X |
| X | | X | X | X | X | X |
| | X | X | X | X | X | X |
| X | X | X | X | X | X | X |

In some embodiments, the medium supplement comprises (1) transferrin, (2) insulin, (3) progesterone, (4) putrescine, (5) selenite, or any combination thereof. Table 3 provides non-limiting examples of combinations of components in the medium supplement.

TABLE 3
Exemplary components of the medium supplement

| (1) | (2) | (3) | (4) | (5) |
|---|---|---|---|---|
| transferrin | insulin | progesterone | putrescine | selenite |
| X | | | | |
| | X | | | |
| X | X | | | |
| | | X | | |
| X | | X | | |
| | X | X | | |
| X | X | X | | |
| | | | X | |
| X | | | X | |
| | X | | X | |
| X | X | | X | |
| | | X | X | |
| X | | X | X | |
| | X | X | X | |
| X | X | X | X | |
| | | | | X |
| X | | | | X |
| | X | | | X |
| X | X | | | X |
| | | X | | X |
| X | | X | | X |
| | X | X | | X |
| X | X | X | | X |
| | | | X | X |
| X | | | X | X |
| | X | | X | X |
| X | X | | X | X |
| | | X | X | X |
| X | | X | X | X |
| | X | X | X | X |
| X | X | X | X | X |

In some embodiments, the salivary gland organoid differentiation medium comprises a medium supplement (see e.g., Table 3) and/or at least one fibroblast growth factor from about day 12 to about day 35-75 after beginning the salivary epithelium differentiation medium (or from about day 0 to about day 23-63 after beginning the salivary gland organoid differentiation medium). In some embodiments, the salivary gland organoid differentiation medium comprises a medium supplement (see e.g., Table 3) and/or at least one fibroblast growth factor on day 12, day 13, day 14, day 15, day 16, day 17, day 18, day 19. day 20, day 21, day 22, day 23, day 24, day 25, day 26, day 27, day 28. day 29, day 30, day 31, day 32. day 33, day 34, day 35, day 36, day 37, day 38, day 39, day 40, day 41, day 42, day 43, day 44. day 45, day 46, day 47, day 48, day 49, day 50, day 51, day 52, day 53. day 54, day 55, day 56, day 57. day 58, day 59, day 60, day 61, day 62. day 63, day 64, day 65, day 66, day 67, day 68, day 69. day 70, day 71, day 72, day 73, day 74. day 75, days 12-35, days 12-45, days 12-55, days 12-65, days 12-75, or any combination thereof, after beginning the salivary epithelium differentiation medium. In some embodiments, the salivary gland organoid differentiation medium comprises a medium supplement (see e.g., Table 3) and/or at least one fibroblast growth factor on day 0, day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, day 31, day 32, day 33, day 34, day 35, day 36, day 37, day 38, day 39, day 40, day 41, day 42, day 43, day 44, day 45, day 46, day 47, day 48, day 49, day 50, day 51, day 52, day 53, day 54, day 55, day 56, day 57, day 58, day 59, day 60, day 61, day 62, day 63, days 0-23, days 0-33, days 0-43, days 0-53, days 0-63, or any combination thereof, after beginning the salivary gland organoid differentiation medium.

In some embodiments, the salivary gland organoid differentiation medium comprises at least one fibroblast growth factor at a concentration from about 125 ng/mL to about 250 ng/mL. In some embodiments, the salivary gland organoid differentiation medium comprises at least one fibroblast growth factor at a concentration of about 50 ng/mL, about 100 ng/mL, about 150 ng/mL, about 200 ng/mL, about 210 ng/mL, about 220 ng/mL, about 230 ng/mL, about 240 ng/mL, about 250 ng/mL, about 260 ng/mL, about 270 ng/mL, about 280 ng/mL, about 290 ng/mL, about 300 ng/mL, about 400 ng/mL, about 500 ng/mL, 240-260 ng/mL, 225-275 ng/mL, 200-300 ng/mL, 125-250 ng/mL, 100-500 ng/mL, or 50-500 ng/mL. In some embodiments, the salivary gland organoid differentiation medium comprises at least one fibroblast growth factor at a concentration of at least 50 ng/mL, at least 100 ng/mL. at least 150 ng/mL, at least 200 ng/mL, at least 210 ng/mL, at least 220 ng/mL, at least 230 ng/mL. at least 240 ng/mL, at least 250 ng/mL, at least 260 ng/mL, at least 270 ng/mL, at least 280 ng/mL. at least 290 ng/mL, at least 300 ng/mL, at least 400 ng/mL, or 500 ng/mL. In some embodiments, the salivary gland organoid differentiation medium comprises at most one fibroblast growth factor at a concentration of at most 50 ng/mL, at most 100 ng/mL, at most 150 ng/mL, at most 200 ng/mL, at most 210 ng/mL, at most 220 ng/mL, at most 230 ng/mL, at most 240 ng/mL, at most 250 ng/mL, at most 260 ng/mL, at most 270 ng/mL, at most 280 ng/mL, at most 290 ng/mL, at most 300 ng/mL, at most 400 ng/mL, or at most 500 ng/mL. In some embodiments, the salivary gland organoid differentiation medium comprises fibroblast growth factor 10 (FGF10) at a concentration of about 250 ng/mL. In some embodiments, the salivary gland organoid differentiation medium comprises fibroblast growth factor 7 (FGF7) at a concentration of about 125 ng/mL. In some embodiments, the salivary gland organoid differentiation medium comprises fibroblast growth factor 1 (FGF1) at a concentration of about 125 ng/mL.

In some embodiments, the at least one fibroblast growth factor is selected from the group consisting of: fibroblast growth factor 10 (FGF10), fibroblast growth factor 7 (FGF7), and fibroblast growth factor 1 (FGF1). In some embodiments, the at least one fibroblast growth factor (FGF) is selected from the group consisting of FGF1, FGF2, FGF3, FGF4, FGF5, FGF6, FGF7, FGF8, FGF9, FGF10, FGF16, FGF17, FGF18, FGF19, FGF20, FGF21, FGF22, and FGF23, or any combination thereof. In some embodiments, the at least one fibroblast growth factor (FGF) is a mammalian fibroblast growth factor (e.g., mammalian FGF1, mammalian FGF7, mammalian FGF10). In some embodiments, the at least one fibroblast growth factor (FGF) is a human fibroblast growth factor (e.g., human FGF1, human FGF7, human FGF10).

In some embodiments, the media supplement comprises an N-2 medium supplement. In some embodiments, the media supplement comprises transferrin at a concentration of about 1000 mg/L (e.g., at a 100× concentration before being diluted to 1× in the salivary gland organoid differentiation medium). In some embodiments, the media supplement comprises transferrin at a concentration of about 10 mg/L (e.g., at a 1× concentration in the salivary gland organoid differentiation medium). In some embodiments, the media supplement comprises transferrin at a concentration of about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L, about 20 mg/L, 9.5-10.5 mg/L, 9-11 mg/L, 7.5-12.5 mg/L, 5-15 mg/L, 1-20 mg/L. In some embodiments, the media supplement comprises transferrin at a concentration of at least 1 mg/L, at least 2 mg/L, at least 3 mg/L. at least 4 mg/L, at least 5 mg/L, at least 6 mg/L, at least 7 mg/L, at least 8 mg/L, at least 9 mg/L, at least 10 mg/L, at least 11 mg/L. at least 12 mg/L, at least 13 mg/L, at least 14 mg/L, at least 15 mg/L, at least 16 mg/L, at least 17 mg/L, at least 18 mg/L, at least 19 mg/L, or 20 mg/L. In some embodiments, the media supplement comprises transferrin at a concentration of at most 1 mg/L, at most 2 mg/L, at most 3 mg/L, at most 4 mg/L, at most 5 mg/L, at most 6 mg/L, at most 7 mg/L, at most 8 mg/L, at most 9 mg/L, at most 10 mg/L, at most 11 mg/L, at most 12 mg/L, at most 13 mg/L, at most 14 mg/L, at most 15 mg/L, at most 16 mg/L, at most 17 mg/L, at most 18 mg/L, at most 19 mg/L, or at most 20 mg/L.

In some embodiments, the media supplement comprises insulin at a concentration of about 500 mg/L (e.g., at a 100× concentration before being diluted to 1× in the salivary gland organoid differentiation medium). In some embodiments, the media supplement comprises insulin at a concentration of about 5 mg/L (e.g., at a 1× concentration in the salivary gland organoid differentiation medium). In some embodiments, the media supplement comprises insulin at a concentration of about 1 mg/L, about 2 mg/L, about 3 mg/L, about 4 mg/L, about 5 mg/L, about 6 mg/L. about 7 mg/L, about 8 mg/L, about 9 mg/L, about 10 mg/L, 4.5-5.5 mg/L, 4-6 mg/L, 2.5-7.5 mg/L. or 1-10 mg/L. In some embodiments, the media supplement comprises insulin at a concentration of at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, or 10 mg/L. In some embodiments, the media supplement comprises insulin at a concentration of at most 1 mg/L, at most 2 mg/L, at most 3 mg/L, at most 4 mg/L, at most 5 mg/L, at most 6 mg/L, at most 7 mg/L, at most 8 mg/L, at most 9 mg/L, or at most 10 mg/L.

In some embodiments, the media supplement comprises progesterone at a concentration of about 0.63 mg/L (e.g., at a 1 00× concentration before being diluted to 1× in the salivary gland organoid differentiation medium). In some embodiments, the media supplement comprises progesterone at a concentration of about 6.3 μg/L (e.g., at a 1× concentration in the salivary gland organoid differentiation medium). In some embodiments, the media supplement comprises progesterone at a concentration of about 1 μg/L, about 2 μg/L, about 3 μg/L, about 4 μg/L, about 5 μg/L, about 6 μg/L, about 7 μg/L, about 8 μg/L, about 9 μg/L, about 10 μg/L, 6-7 μg/L, 5.5-6.5 μg/L, 5-7 μg/L, 2.5-7.5 μg/L, or 1-10 μg/L. In some embodiments, the media supplement comprises progesterone at a concentration of at least 6.3 μg/L. In some embodiments, the media supplement comprises progesterone at a concentration of at least 1 μg/L, at least 2 μg/L, at least 3 μg/L, at least 4 μg/L, at least 5 μg/L, at least 6 μg/L, at least 7 μg/L, at least 8 μg/L, at least 9 μg/L, or 10 μg/L. In some embodiments, the media supplement comprises progesterone at a concentration of at most 6.3 μg/L. In some embodiments, the media supplement comprises progesterone at a concentration of at most 1 μg/L, at most 2 μg/L, at most 3 μg/L, at most 4 μg/L, at most 5 μg/L, at most 6 μg/L, at most 7 μg/L, at most 8 μg/L, at most 9 μg/L, or at most 10 μg/L.

In some embodiments, the media supplement comprises putrescine at a concentration of about 1611 mg/L (e.g.. at a 100× concentration before being diluted to 1× in the salivary gland organoid differentiation medium). (e.g.. at a 1× concentration in the salivary gland organoid differentiation medium). In some embodiments, the media supplement comprises putrescine at a concentration of about 16.11 mg/L. In some embodiments, the media supplement comprises putrescine at a concentration of about 10 mg/L, about 11 mg/L, about 12 mg/L, about 13 mg/L, about 14 mg/L, about 15 mg/L, about 16 mg/L, about 16.1 mg/L, about 16.2 mg/L, about 16.5 mg/L, about 17 mg/L, about 18 mg/L, about 19 mg/L, about 20 mg/L, 16-16.5 mg/L, 15.5-16.5 mg/L, 15-17 mg/L, or 10-20 mg/L. In some embodiments, the media supplement comprises putrescine at a concentration of at least 16.11 mg/L. In some embodiments, the media supplement comprises putrescine at a concentration of at least 10 mg/L, at least 11 mg/L, at least 12 mg/L, at least 13 mg/L, at least 14 mg/L. at least 15 mg/L, at least 16 mg/L, at least 16.1 mg/L, at least 16.2 mg/L, at least 16.5 mg/L, at least 17 mg/L. at least 18 mg/L, at least 19 mg/L, or 20 mg/L. In some embodiments, the media supplement comprises putrescine at a concentration of at most 16.11 mg/L. In some embodiments, the media supplement comprises putrescine at a concentration of at most 10 mg/L, at most 11 mg/L, at most 12 mg/L, at most 13 mg/L, at most 14 mg/L. at most 15 mg/L, at most 16 mg/L, at most 16.1 mg/L. at most 16.2 mg/L, at most 16.5 mg/L, at most 17 mg/L. at most 18 mg/L, at most 19 mg/L, or at most 20 mg/L.

In some embodiments, the media supplement comprises selenite at a concentration of about 0.52 mg/L (e.g., at a 100× concentration before being diluted to 1× in the salivary gland organoid differentiation medium). In some embodiments, the media supplement comprises selenite at a concentration of about 5.2 μg/L (e.g., at a 1× concentration in the salivary gland organoid differentiation medium). In some embodiments, the media supplement comprises selenite at a concentration of about 1 μg/L, about 2 μg/L, about 3 μg/L, about 4 μg/L, about 5 μg/L, about 6 μg/L, about 7 μg/L, about 8 μg/L, about 9 μg/L, about 10 μg/L, 5-6 μg/L. 5.5-6.5 μg/L, 5-7 μg/L, 2.5-7.5 μg/L, or 1-10 μg/L. In some embodiments, the media supplement comprises selenite at a concentration of at least 5.2 μg/L. In some embodiments, the media supplement comprises selenite at a concentration of at least 1 μg/L, at least 2 μg/L, at least 3 μg/L, at least 4 μg/L, at least 5 μg/L, at least 6 μg/L, at least 7 μg/L, at least 8 μg/L, at least 9 μg/L, or 10 pg/L. In some embodiments, the media supplement comprises selenite at a concentration of at most 5.2 μg/L. In some embodiments, the media supplement comprises selenite at a concentration of at most 1 pg/L, at most 2 μg/L, at most 3 μg/L, at most 4 μg/L, at most 5 μg/L, at most 6 μg/L, at most 7 pg/L, at most 8 μg/L, at most 9 gg/L, or at most 10 μg/L.

In some embodiments, the salivary gland organoid differentiation medium comprises a neuregulin from about day 18 to about day 35-75 after beginning the salivary epithelium differentiation medium (or from about day 6 to about day 23-63 after beginning the salivary gland organoid differentiation medium). In some embodiments, the salivary gland organoid differentiation medium comprises a neuregulin on day 18, day 19, day 20, day 21. day 22, day 23, day 24, day 25. day 26, day 27, day 28, day 29. day 30, day 31, day 32, day 33, day 34. day 35, day 36, day 37, day 38, day 39, day 40, day 41. day 42, day 43, day 44, day 45, day 46. day 47, day 48, day 49, day 50. day 51, day 52, day 53, day 54. day 55. day 56, day 57, day 58, day 59. day 60, day 61, day 62, day 63, day 64, day 65, day 66. day 67. day 68, day 69, day 70, day 71. day 72, day 73, day 74, day 75. days 18-35, days 18-45, days 18-55, days 18-65, days 18-75, or any combination thereof, after beginning the salivary epithelium differentiation medium. In some embodiments, the salivary gland organoid differentiation medium comprises a neuregulin on day 6, day 7, day 8, day 9, day 10, day 11, day 12, day 13, day 14, day 15, day 16, day 17, day 18, day 19, day 20, day 21, day 22, day 23, day 24, day 25, day 26, day 27. day 28, day 29, day 30, day 31, day 32, day 33, day 34, day 35, day 36. day 37, day 38, day 39, day 40, day 41, day 42, day 43, day 44, day 45, day 46, day 47, day 48, day 49, day 50, day 51, day 52, day 53, day 54, day 55, day 56, day 57, day 58, day 59, day 60, day 61, day 62, day 63, days 6-23, days 6-33, days 6-43, days 6-53, days 6-63, or any combination thereof, after beginning the salivary gland organoid differentiation medium.

In some embodiments, the salivary gland organoid differentiation medium comprises a neuregulin at a concentration of about 250 ng/mL. In some embodiments, the salivary gland organoid differentiation medium comprises a neuregulin at a concentration of about 50 ng/mL, about 100 ng/mL, about 150 ng/mL, about 200 ng/mL, about 210 ng/mL, about 220 ng/mL, about 230 ng/mL, about 240 ng/mL, about 250 ng/mL, about 260 ng/mL, about 270 ng/mL, about 280 ng/mL, about 290 ng/mL, about 300 ng/mL, about 400 ng/mL, about 500 ng/mL, 240-260 ng/mL, 225-275 ng/mL, 200-300 ng/mL, 100-500 ng/mL, or 50-500 ng/mL. In some embodiments, the salivary gland organoid differentiation medium comprises a neuregulin at a concentration of at least 50 ng/mL, at least 100 ng/mL, at least 150 ng/mL, at least 200 ng/mL, at least 210 ng/mL, at least 220 ng/mL, at least 230 ng/mL, at least 240 ng/mL, at least 250 ng/mL, at least 260 ng/mL, at least 270 ng/mL, at least 280 ng/mL, at least 290 ng/mL, at least 300 ng/mL, at least 400 ng/mL, or 500 ng/mL. In some embodiments, the salivary gland organoid differentiation medium comprises a neuregulin at a concentration of at most 50 ng/mL, at most 100 ng/mL, at most 150 ng/mL, at most 200 ng/nL, at most 210 ng/mL, at most 220 ng/mL, at most 230 ng/mL, at most 240 ng/mL, at most 250 ng/mL, at most 260 ng/mL, at most 270 ng/mL, at most 280 ng/mL, at most 290 ng/mL, at most 300 ng/mL, at most 400 ng/mL, or at most 500 ng/mL. In some embodiments, the neuregulin is Neuregulin 1 (NRG1). In some embodiments, the neuregulin is mammalian Neuregulin 1 (NRG1). In some embodiments, the neuregulin is human Neuregulin 1 (NRG1). In some embodiments, the neuregulin is NRG1, NRG2, NRG3, and/or NRG4.

In some embodiments, the salivary gland organoid differentiation medium comprises an extracellular matrix glycoprotein and/or a basement membrane matrix from about day 18 to about day 24-75 after beginning the salivary epithelium differentiation medium (or from about day 6 to about day 12-63 after beginning the salivary gland organoid differentiation medium). In some embodiments, the salivary gland organoid differentiation medium comprises an extracellular matrix glycoprotein and/or a basement membrane matrix on day 18, day 19, day 20, day 21, day 22, day 23, day 24, day 25, day 26, day 27, day 28. day 29. day 30, day 31, day 32, day 33, day 34, day 35, day 36, day 37, day 38, day 39, day 40, day 41, day 42, day 43, day 44, day 45, day 46, day 47, day 48, day 49, day 50, day 51, day 52, day 53, day 54, day 55, day 56, day 57, day 58, day 59, day 60, day 61, day 62, day 63, day 64, day 65, day 66, day 67, day 68, day 69, day 70, day 71, day 72, day 73, day 74, day 75, days 18-24, days 18-25, days 18-35, days 18-45, days 18-55, days 18-65, days 18-75, or any combination thereof, after beginning the salivary epithelium differentiation medium. In some embodiments, the salivary gland organoid differentiation medium comprises an extracellular matrix glycoprotein and/or a basement membrane matrix on day 6, day 7, day 8, day 9, day 10, day 11, day 12, day 13, day 14, day 15. day 16, day 17, day 18, day 19, day 20, day 21, day 22, day 23, day 24. day 25, day 26, day 27, day 28. day 29, day 30, day 31, day 32, day 33. day 34, day 35, day 36, day 37, day 38, day 39, day 40. day 41, day 42, day 43, day 44, day 45. day 46, day 47, day 48, day 49. day 50, day 51, day 52, day 53. day 54, day 55, day 56, day 57, day 58. day 59, day 60, day 61, day 62, day 63, days 6-12, days 6-13. days 6-23, days 6-33, days 6-43, days 6-53. days 6-63. or any combination thereof, after beginning the salivary gland organoid differentiation medium.

In some embodiments, the salivary gland organoid differentiation medium comprises an extracellular matrix glycoprotein at a concentration of about 1%. In some embodiments, the salivary gland organoid differentiation medium comprises an extracellular matrix glycoprotein at a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%. 0.9-1.1%. 0.5-1.5%, 0.1-2%, or 0.1-5%. In some embodiments, the salivary gland organoid differentiation medium comprises an extracellular matrix glycoprotein at a concentration of at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or 5%. In some embodiments, the salivary gland organoid differentiation medium comprises an extracellular matrix glycoprotein at a concentration of at most 0.1%, at most 0.2%, at most 0.3%, at most 0.4%, at most 0.5%, at most 0.6%, at most 0.7%, at most 0.8%, at most 0.9%, at most 1%, at most 2%, at most 3%, at most 4%, or at most 5%. In some embodiments, the extracellular matrix glycoprotein is a laminin. In some embodiments, the extracellular matrix glycoprotein is a mammalian laminin. In some embodiments, the extracellular matrix glycoprotein is a human laminin. In some embodiments, the laminin is selected from the group consisting of Laminin-111. Laminin-211, Laminin-121, Laminin-221, Laminin-332/Laminin-3A32, Laminin-3B32, Laminin-311/Laminin-3A11, Laminin-321/Laminin-3A21, Laminin-411, Laminin-421, Laminin-511. Laminin-521, Laminin-213, Laminin-423, Laminin-522, and Laminin-523, or any combinations thereof. In some embodiments, the extracellular matrix glycoprotein is selected from the group consisting of a fibronectin. a laminin, and a matricellular glycoprotein (e.g., Tenascin. Osteopontin, Fibulin).

In some embodiments, the salivary gland organoid differentiation medium comprises a basement membrane matrix at a concentration of about 3%. In some embodiments, the salivary gland organoid differentiation medium comprises a basement membrane matrix at a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, 0.9-1.1%, 0.5-1.5%, 0.1-2%, or 0.1-5%. In some embodiments, the salivary gland organoid differentiation medium comprises a basement membrane matrix at a concentration of at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or 5%. In some embodiments, the salivary gland organoid differentiation medium comprises a basement membrane matrix at a concentration of at most 0.1%, at most 0.2%, at most 0.3%, at most 0.4%, at most 0.5%, at most 0.6%, at most 0.7%, at most 0.8%, at most 0.9%, at most 1%, at most 2%, at most 3%, at most 4%, or at most 5%. In some embodiments, the basement membrane matrix is secreted by Engelbreth-Holm-Swarm (EHS) mouse sarcoma cells (MATRIGEL®). In some embodiments, the basement membrane matrix comprises collagen (e.g., type IV), laminin, heparan sulfate, entactin, and/or fibronectin, or any combinations thereof. In some embodiments, the basement membrane matrix is growth factor reduced (GFR). In some embodiments, the basement membrane matrix comprises essentially no growth factors, non-limiting examples of which include vascular endothelial growth factor (VEGF), epidermal growth factor (EGF), and/or platelet-derived growth factor (PDGF).

In some embodiments, the salivary gland organoid differentiation medium comprises an inhibitor of Rho Associated Coiled-Coil Containing Protein Kinase (ROCK) from about day 24 to about day 27 after beginning the salivary epithelium differentiation medium (or from about day 12 to about day 15 after beginning the salivary gland organoid differentiation medium). In some embodiments, the salivary gland organoid differentiation medium comprises an inhibitor of Rho Associated Coiled-Coil Containing Protein Kinase (ROCK) on day 24, day 25, day 26, day 27, days 24-25, days 25-26, days 26-27, day 24-26, day 25-27, days 24-27, or any combination thereof, after beginning the salivary epithelium differentiation medium. In some embodiments, the salivary gland organoid differentiation medium comprises an inhibitor of Rho Associated Coiled-Coil Containing Protein Kinase (ROCK) on day 12, day 13, day 14, day 15, days 12-13, days 13-14, days 14-15, days 12-14, days 13-15, days 12-15, or any combination thereof, after beginning the salivary gland organoid differentiation medium.

In some embodiments, the salivary gland organoid differentiation medium comprises an inhibitor of Rho Associated Coiled-Coil Containing Protein Kinase (ROCK) at a concentration of about 50 μM. In some embodiments, the inhibitor of ROCK is at a concentration of about 10 μM, about 20 μM, about 30 μM, about 40 μM, about 45 μM, about 50 μM, about 55 μM, about 60 μM, about 70 μM, about 80 μM, about 90 μM, about 100 μM, about 45 μM to about 55 μM, about 40 μM to about 60 μM, about 35 μM to about 65 μM. about 30 μM to about 70 μM, about 25 μM to about 75 μM, about 20 μM to about 80 μM, about 15 μM to about 85 μM, about 10 μM to about 90 μM about 5 μM to about 95 μM, or about 1 μM to about 100 μM. In some embodiments, the inhibitor of ROCK is at a concentration of at least 10 μM, at least 20 μM, at least 30 μM, at least 40 μM, at least 45 μM, at least 50 μM, at least 55 μM, at least 60 μM, at least 70 μM, at least 80 μM, at least 90 μM, or 100 μM. In some embodiments, the inhibitor of ROCK is at a concentration of at most 10 μM, at most 20 μM, at most 30 μM, at most 40 μM, at most 45 μM, at most 50 μM, at most 55 μM, at most 60 μM, at most 70 μM, at most 80 μM, at most 90 μM, or at most 100 μM. In some embodiments, the inhibitor of ROCK is Y-27632. In some embodiments, the inhibitor of ROCK is selected from the group consisting of AT-13148, BA-210,β-Elemene, Belumosudil, Chroman, DJ4. GSK-576371. GSK429286A (C21H16F4N402), H-1152, HA-1077 (Fasudil), Hydroxyfasudil (an active metabolite of fasudil), Ibuprofen, LX-7101, Netarsudil, RKI-1447, Ripasudil, TCS-7001, Thiazovivin, Verosudil (AR-12286), Y-27632. Y-30141. Y-33075, and Y-39983, or analogs or combinations thereof.

In some embodiments, the salivary gland organoid differentiation medium comprises a growth factor for epithelium: an inhibitor of a Glycogen Synthase Kinase (GSK) and/or activator of a Wingless/Integrated (Wnt) and D-catenin pathway; and/or an inhibitor of transforming growth factor-D (TGF-β) type I receptor from about day 24 to about day 35-75 after beginning the salivary epithelium differentiation medium (or from about day 12 to about day 23-63 after beginning the salivary gland organoid differentiation medium). In some embodiments, the salivary gland organoid differentiation medium comprises a growth factor for epithelium; an inhibitor of a Glycogen Synthase Kinase (GSK) and/or activator of a Wingless/Integrated (Wnt) and β-catenin pathway; and/or an inhibitor of transforming growth factor-β (TGF-β) type I receptor on day 24, day 25, day 26, day 27, day 28, day 29, day 30, day 31, day 32, day 33, day 34, day 35, day 36, day 37, day 38, day 39, day 40, day 41, day 42, day 43, day 44, day 45, day 46, day 47, day 48, day 49, day 50, day 51, day 52, day 53, day 54, day 55, day 56, day 57, day 58, day 59, day 60, day 61, day 62, day 63, day 64, day 65, day 66, day 67, day 68, day 69, day 70, day 71, day 72, day 73, day 74, day 75, days 24-35, days 24-45, days 24-55, days 24-65, days 24-75, or any combination thereof, after beginning the salivary epithelium differentiation medium. In some embodiments, the salivary gland organoid differentiation medium comprises a growth factor for epithelium; an inhibitor of a Glycogen Synthase Kinase (GSK) and/or activator of a Wingless/Integrated (Wnt) and β-catenin pathway; and/or an inhibitor of transforming growth factor-β (TGF-β) type I receptor on day 12, day 13, day 14, day 15, day 16, day 17, day 18, day 19, day 20, day 21, day 22. day 23, day 24, day 25, day 26, day 27, day 28, day 29, day 30, day 31, day 32, day 33, day 34. day 35, day 36, day 37, day 38, day 39, day 40, day 41, day 42, day 43. day 44, day 45, day 46, day 47. day 48, day 49, day 50, day 51, day 52, day 53, day 54, day 55, day 56, day 57, day 58, day 59. day 60, day 61, day 62, day 63, days 12-23. days 12-33, days 12-43, days 12-53, days 12-63. or any combination thereof, after beginning the salivary gland organoid differentiation medium.

In some embodiments, the salivary gland organoid differentiation medium comprises a growth factor for epithelium at a concentration of about 100 ng/mL. In some embodiments, the salivary gland organoid differentiation medium comprises a growth factor for epithelium at a concentration of about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, about 200 ng/mL, about 300 ng/mL, about 400 ng/mL, about 500 ng/mL, 90-110 ng/mL, 75-125 ng/mL, 50-150 ng/mL, 10-200 ng/mL, or 10-500 ng/mL. In some embodiments, the salivary gland organoid differentiation medium comprises a growth factor for epithelium at a concentration of at least 10 ng/mL, at least 20 ng/mL, at least 30 ng/mL, at least 40 ng/mL, at least 50 ng/mL, at least 60 ng/mL, at least 70 ng/mL, at least 80 ng/mL, at least 90 ng/mL, at least 100 ng/mL. at least 110 ng/mL, at least 120 ng/mL, at least 130 ng/mL, at least 140 ng/mL, at least 150 ng/mL. at least 160 ng/mL, at least 170 ng/mL, at least 180 ng/mL, at least 190 ng/mL, at least 200 ng/mL. at least 300 ng/mL, at least 400 ng/mL, or 500 ng/mL. In some embodiments, the salivary gland organoid differentiation medium comprises a growth factor for epithelium at a concentration of at most 10 ng/mL, at most 20 ng/mL, at most 30 ng/mL, at most 40 ng/mL, at most 50 ng/mL, at most 60 ng/mL, at most 70 ng/mL, at most 80 ng/mL, at most 90 ng/mL, at most 100 ng/mL, at most 110 ng/mL, at most 120 ng/mL, at most 130 ng/mL, at most 140 ng/mL, at most 150 ng/mL, at most 160 ng/mL, at most 170 ng/mL, at most 180 ng/mL, at most 190 ng/mL, at most 200 ng/mL, at most 300 ng/mL, at most 400 ng/mL, or at most 500 ng/mL. In some embodiments, the growth factor for epithelium is epithelial epidermal growth factor (EGF). In some embodiments, the growth factor for epithelium is mammalian epidermal growth factor (EGF). In some embodiments, the growth factor for epithelium is human epidermal growth factor (EGF). In some embodiments, the growth factor for epithelium is selected from the group consisting of Heparin Binding EGF Like Growth Factor (HB-EGF), Transforming Growth Factor Alpha (TGF-α), epigen, neuregulin (e.g., NRG1, NRG2, NRG3, NRG4), amphiregulin, an EGF-Like Protein (e.g., EGFL6, EGFL7, EGFL8), betacellulin, and a tomoregulin (e.g., TMEFF1, TMEFF2).

In some embodiments, the salivary gland organoid differentiation medium comprises an inhibitor of a Glycogen Synthase Kinase (GSK) and/or activator of a Wingless/Integrated (Wnt) and β-catenin pathway at a concentration of about 5 μM. In some embodiments, the salivary gland organoid differentiation medium comprises an inhibitor of a Glycogen Synthase Kinase (GSK) and/or activator of a Wingless/Integrated (Wnt) and β-catenin pathway at a concentration of about 0.5 μM, about 1 μM, about 2 μM, about 3 μM, about 4 μM, about 5 μM, about 6 μM, about 7 μM, about 8 μM, about 9 μM, about 10 μM, 4.5-5.5 μM, 4-6 μM, 3-7 pM, 2-8 μM, 1-9 μM, or 0.5-10 μM. In some embodiments, the salivary gland organoid differentiation medium comprises an inhibitor of a Glycogen Synthase Kinase (GSK) and/or activator of a Wingless/Integrated (Wnt) and β-catenin pathway at a concentration of at least 0.5 μM, at least 1 μM, at least 2 μM, at least 3 μM, at least 4 μM, at least 5 μM, at least 6 μM, at least 7 μM, at least 8 μM, at least 9 μM, or 10 μM. In some embodiments, the salivary gland organoid differentiation medium comprises an inhibitor of a Glycogen Synthase Kinase (GSK) and/or activator of a Wingless/Integrated (Wnt) and β-catenin pathway at a concentration of at most 0.5 μM, at most 1 μM, at most 2 μM, at most 3 μM, at most 4 μM, at most 5 μM, at most 6 μM, at most 7 μM, at most 8 μM, at most 9 μM, or at most 10 μM. In some embodiments, the inhibitor of GSK and/or activator of the Wnt and β-catenin pathway is Chiron (Chir99021). In some embodiments, the inhibitor of GSK and/or activator of the Wnt and β-catenin pathway is selected from the group consisting of Chir99021, cromolyn sodium, lithium chloride, azakenpaullone (1-Akp), tideglusib, 6-bromoindirubin-3, Wnt-1, Wnt-2, Wnt-2b, Wnt-3a, Wnt-4, Wnt-5a. Wnt-5b, Wnt-6, Wnt-7a. Wnt-7a/b. R-spondin (RSPO), and Norrin, or analogs or combinations thereof.

In some embodiments, the salivary gland organoid differentiation medium comprises an inhibitor of transforming growth factor-(3 (TGF-β) type I receptor at a concentration of about 10 μM. In some embodiments, the salivary gland organoid differentiation medium comprises an inhibitor of transforming growth factor-β (TGF-β) type I receptor at a concentration of about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, 9-11 μM, 7.5-12.5 μM, 5-15 μM, or 1-20 μM. In some embodiments, the salivary gland organoid differentiation medium comprises an inhibitor of transforming growth factor-β (TGF-β) type I receptor at a concentration of at least 1 μM, at least 2 μM, at least 3 μM, at least 4 μM, at least 5 μM, at least 6 μM, at least 7 μM, at least 8 μM, at least 9 μM, at least 10 μM, at least 11 μM, at least 12 μM, at least 13 μM, at least 14 μM, at least 15 μM, at least 16 μM, at least 17 μM, at least 18 μM, at least 19 μM, or 20 μM. In some embodiments, the salivary gland organoid differentiation medium comprises an inhibitor of transforming growth factor-β (TGF-β) type I receptor at a concentration of at most 1 μM, at most 2 μM, at most 3 μM, at most 4 μM, at most 5 μM, at most 6 μM, at most 7 μM, at most 8 μM, at most 9 μM, at most 10 μM, at most 11 μM, at most 12 μM, at most 13 μM, at most 14 μM, at most 15 μM, at most 16 μM, at most 17 μM, at most 18 μM, at most 19 μM, or at most 20 μM. In some embodiments, the inhibitor of the TGF-β type I receptor is SB-431452. In some embodiments, the inhibitor of the TGF-β type I receptor is selected from the group consisting of vactosertib, GW788388, RepSox (E-616452), SB-431542, Galunisertib (LY2157299), and LY364947.

In some embodiments, the salivary gland organoid differentiation medium comprises Dulbecco's Modified Eagle Medium and Nutrient Mixture F12 (DMEM/F12™) medium. The components of DMEM/F12 medium include, but are not limited to: Amino Acids including but not limited to: Glycine, L-Alanine. L-Arginine hydrochloride, L-Asparagine-H₂O, L-Aspartic acid, L-Cysteine hydrochloride-H₂O, L-Cystine 2HCl, L-Glutamic Acid, L-Glutamine. L-Histidine hydrochloride-H₂O. L-Isoleucine, L-Leucine. L-Lysine hydrochloride. L-Methionine. L-Phenylalanine, L-Proline, L-Serine, L-Threonine, L-Tryptophan, L-Tyrosine disodium salt dihydrate, and/or L-Valine, and Vitamins including but not limited to: Biotin, Choline chloride, D-Calcium pantothenate, and/or Folic Acid.

In some embodiments, the sufficient amount of time to promote differentiation of salivary epithelium into the salivary gland organoid culture is about 23-63 days. In some embodiments, the sufficient amount of time to promote differentiation of salivary epithelium into the salivary gland organoid culture is about 38 days. In some embodiments, the sufficient amount of time to promote differentiation of salivary epithelium into the salivary gland organoid culture is about 23 days, about 24 days, about 25 days, about 26 days, about 27 days, about 28 days, about 29 days, about 30 days, about 31 days. about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days. about 52 days, about 53 days, about 54 days, about 55 days, about 56 days, about 57 days, about 58 days, about 59 days, about 60 days, about 61 days, about 62 days, or about 63 days. In some embodiments, the sufficient amount of time to promote differentiation of salivary epithelium into the salivary gland organoid culture is at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, at least 51 days, at least 52 days, at least 53 days, at least 54 days, at least 55 days, at least 56 days, at least 57 days, at least 58 days, at least 59 days, at least 60 days, at least 61 days, at least 62 days, or 63 days. In some embodiments, the sufficient amount of time to promote differentiation of salivary epithelium into the salivary gland organoid culture is at most 23 days, at most 24 days, at most 25 days, at most 26 days, at most 27 days, at most 28 days, at most 29 days, at most 30 days, at most 31 days, at most 32 days, at most 33 days, at most 34 days, at most 35 days, at most 36 days, at most 37 days, at most 38 days, at most 39 days, at most 40 days, at most 41 days, at most 42 days, at most 43 days, at most 44 days, at most 45 days, at most 46 days, at most 47 days, at most 48 days, at most 49 days, at most 50 days, at most 51 days, at most 52 days, at most 53 days, at most 54 days, at most 55 days, at most 56 days, at most 57 days, at most 58 days, at most 59 days, at most 60 days, at most 61 days, at most 62 days, or at most 63 days.

In some embodiments, the sufficient amount of time to promote differentiation of the population of pluripotent stem cells into the salivary gland organoid culture is about 53 days. In some embodiments, the sufficient amount of time to promote differentiation of the pluripotent stem cell clusters into the salivary gland organoid culture is about 50 days. In some embodiments, the sufficient amount of time to promote differentiation of the pluripotent stem cell clusters into the salivary gland organoid culture is about 45 days, about 46 days, about 47 days, about 48 days, about 49 days, about 50 days, about 51 days, about 52 days, about 53 days, about 54 days, or about 55 days. In some embodiments, the sufficient amount of time to promote differentiation of the pluripotent stem cell clusters into the salivary gland organoid culture is at least 45 days, at least 46 days, at least 47 days, at least 48 days, at least 49 days, at least 50 days, at least 51 days, at least 52 days, at least 53 days, at least 54 days, or 55 days. In some embodiments, the sufficient amount of time to promote differentiation of the pluripotent stem cell clusters into the salivary gland organoid culture is at most 45 days, at most 46 days, at most 47 days, at most 48 days, at most 49 days, at most 50 days. at most 51 days, at most 52 days, at most 53 days, at most 54 days, or at most 55 days.

In some embodiments, the sufficient amount of time to promote differentiation of the pluripotent stem cell clusters into the salivary gland organoid culture is about 30 days to 50 days. In some embodiments, the sufficient amount of time to promote differentiation of the pluripotent stem cell clusters into the salivary gland organoid culture is about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, or about 50 days. In some embodiments, the sufficient amount of time to promote differentiation of the pluripotent stem cell clusters into the salivary gland organoid culture is at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, or 50 days. In some embodiments, the sufficient amount of time to promote differentiation of the pluripotent stem cell clusters into the salivary gland organoid culture is at most 30 days, at most 31 days, at most 32 days, at most 33 days, at most 34 days, at most 35 days, at most 36 days, at most 37 days, at most 38 days, at most 39 days, at most 40 days, at most 41 days, at most 42 days, at most 43 days, at most 44 days, at most 45 days, at most 46 days, at most 47 days, at most 48 days, at most 49 days, or at most 50 days.

In some embodiments, the salivary gland organoid culture secretes salivary amylase after about day 35 after beginning the salivary epithelium differentiation medium. In some embodiments, the salivary gland organoid culture secretes salivary amylase after about day 24, day 25, day 26, day 27, day 28, day 29, day 30. day 31, day 32, day 33, day 34, day 35, day 36, day 37, day 38, day 39. day 40, day 41, day 42, day 43. day 44, day 45, day 46, day 47, day 48, day 49, day 50, days 25-35, days 25-45, days 25-50, days 35-45, days 35-50, or any combination thereof, after beginning the salivary epithelium differentiation medium.

In some embodiments, the salivary gland organoid differentiation medium further comprises a sugar or sugar alcohol and/or at least one proinflammatory cytokine from at least about day 35 after beginning the salivary epithelium differentiation medium (or from at least about day 27 after beginning the salivary gland organoid differentiation medium). In some embodiments, the salivary gland organoid differentiation medium further comprises a sugar or sugar alcohol and/or at least one proinflammatory cytokine on day 35, day 36, day 37, day 38, day 39, day 40, day 41, day 42, day 43, day 44, day 45, day 46, day 47, day 48, day 49, day 50, day 51, day 52, day 53, day 54, day 55, day 56, day 57, day 58. day 59, day 60, day 61, day 62, day 63, day 64, day 65, day 66, day 67, day 68, day 69, day 70. day 71, day 72, day 73, day 74, day 75, days 35-45, days 35-55, days 35-65, days 35-75, or any combination thereof, after beginning the salivary epithelium differentiation medium. In some embodiments, the salivary gland organoid differentiation medium further comprises a sugar or sugar alcohol and/or at least one proinflammatory cytokine on day 27, day 28, day 29. day 30, day 31, day 32, day 33. day 34, day 35, day 36, day 37, day 38. day 39, day 40, day 41, day 42. day 43, day 44, day 45, day 46. day 47. day 48, day 49, day 50, day 51. day 52, day 53, day 54, day 55, day 56, day 57, day 58. day 59. day 60, day 61, day 62, day 63. days 27-33, days 27-43, days 27-53, days 27-63, or any combination thereof, after beginning the salivary gland organoid differentiation medium.

In some embodiments, the salivary gland organoid differentiation medium comprises a sugar or sugar alcohol at a concentration of about 53-70 mM. In some embodiments, the salivary gland organoid differentiation medium comprises a sugar or sugar alcohol at a physiological concentration, e.g., the level in a sample (e.g., blood, plasma, serum, saliva) from a healthy, non-diabetic subject. In some embodiments, the salivary gland organoid differentiation medium comprises a sugar or sugar alcohol at a concentration of about 1.0 g/L or about 9.25 mM. In some embodiments, the salivary gland organoid differentiation medium comprises a sugar or sugar alcohol at a concentration of about 17.5 mM. In some embodiments, the salivary gland organoid differentiation medium comprises a sugar or sugar alcohol at a concentration of about 4.5 g/L or about 41.6 mM. In some embodiments, the salivary gland organoid differentiation medium comprises a sugar or sugar alcohol at a concentration of about 9 mM, about 10 mM, about 15 mM, about 17.5 mM, about 20 mM, about 30 mM, about 40 mM, about 50 mM, about 55 mM, about 60 mM, about 65 mM, about 70 mM, 9-10 mM, 9-20 mM, 35-45 mM, 50-70 mM, 55-70 mM, 50-60 mM, 55-70 mM, or 60-70 mM. In some embodiments, the salivary gland organoid differentiation medium comprises a sugar or sugar alcohol at a concentration of at least 9 mM, at least 10 mM, at least 15 mM, at least 17.5 mM, at least 20 mM, at least 30 mM, at least 40 mM, at least 50 mM, at least 55 mM, at least 60 mM, at least 65 mM, or 70 mM. In some embodiments, the salivary gland organoid differentiation medium comprises a sugar or sugar alcohol at a concentration of at most 9 mM, at most 10 mM, at most 15 mM, at most 17.5 mM, at most 20 mM, at most 30 mM, at most 40 mM, at most 50 mM, at most 55 mM, at most 60 mM, at most 65 mM, or at most 70 mM. In some embodiments, the sugar comprises glucose. In some embodiments, the sugar comprises D-glucose. In some embodiments, the sugar alcohol comprises D-mannitol. In some embodiments, the sugar alcohol comprises mannitol.

In some embodiments, the salivary gland organoid differentiation medium comprises at least one proinflammatory cytokine at a concentration of about 1 ng/nl. In some embodiments, the salivary gland organoid differentiation medium comprises at least one proinflammatory cytokine at a concentration of about 0.1 ng/mL, about 0.2 ng/mL, about 0.3 ng/mL, about 0.4 ng/mL, about 0.5 ng/mL, about 0.6 ng/mL, about 0.7 ng/mL, about 0.8 ng/mL, about 0.9 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, 0.9-1.1 ng/mL, 0.75-1.25 ng/mL, 0.5-1.5 ng/mL, 0.1-2 ng/mL, 0.1-5 ng/mL, or 0.1-10 ng/mL. In some embodiments, the salivary gland organoid differentiation medium comprises at least one proinflammatory cytokine at a concentration of at least 0.1 ng/mL, at least 0.2 ng/mL, at least 0.3 ng/mL, at least 0.4 ng/mL, at least 0.5 ng/mL, at least 0.6 ng/mL, at least 0.7 ng/mL, at least 0.8 ng/mL, at least 0.9 ng/mL, at least 1 ng/mL, at least 2 ng/mL, at least 3 ng/mL, at least 4 ng/mL, at least 5 ng/mL, at least 6 ng/mL, at least 7 ng/mL, at least 8 ng/mL, at least 9 ng/mL, or 10 ng/mL. In some embodiments, the salivary gland organoid differentiation medium comprises at most one proinflammatory cytokine at a concentration of at most 0.1 ng/mL, at most 0.2 ng/mL, at most 0.3 ng/mL, at most 0.4 ng/mL, at most 0.5 ng/mL, at most 0.6 ng/mL, at most 0.7 ng/mL, at most 0.8 ng/mL. at most 0.9 ng/mL, at most 1 ng/mL, at most 2 ng/mL. at most 3 ng/mL, at most 4 ng/mL, at most 5 ng/mL, at most 6 ng/mL, at most 7 ng/mL, at most 8 ng/mL, at most 9 ng/mL, or at most 10 ng/mL.

In some embodiments, the at least one proinflammatory cytokine is TNFa and/or IL-6. In some embodiments, the at least one proinflammatory cytokine is selected from the group consisting of IL-1, IL-2, IL-6, IL-12, IL-17, IL-18, IFN-γ, and TNF-α. In some embodiments, the at least one proinflammatory cytokine is a mammalian proinflammatory cytokine. In some embodiments, the at least one proinflammatory cytokine is a human proinflammatory cytokine.

### Salivary Gland Organoid Culture Media

In one aspect, described herein are salivary gland organoid media. In some aspects and embodiments, a salivary gland organoid culture as described herein is in combination with such a salivary gland organoid culture.

In some embodiments, the salivary gland organoid medium comprises: (a) medium supplement: (b) at least one fibroblast growth factor; (c) a neuregulin; (d) an extracellular matrix glycoprotein; (e) a basement membrane matrix: (f) a growth factor for epithelium: (g) an inhibitor of a Glycogen Synthase Kinase (GSK) and/or activator of a Wingless/Integrated (Wnt) and β-catenin pathway: (h) an inhibitor of transforming growth factor-β (TGF-β) type I receptor: or any combination thereof. In some embodiments, the salivary gland organoid medium comprises (a) a medium supplement and at least one of the following: (b) at least one fibroblast growth factor; (c) a neuregulin; (d) an extracellular matrix glycoprotein; (e) a basement membrane matrix; (f) a growth factor for epithelium; (g) an inhibitor of a Glycogen Synthase Kinase (GSK and/or activator of a Wingless/Integrated (Wnt) and d-catenin pathway; (h) an inhibitor of transforming growth factor-β (TGF-β) type I receptor; or any combinations thereof. Table 4 provides non-limiting examples of combinations of components in salivary gland organoid medium. The concentrations of the components of the salivary gland organoid medium can be the same or different as those described in the salivary gland organoid differentiation media.

TABLE 4
Exemplary components of the salivary gland organoid medium (each can be
in combination with (a) a medium supplement as described herein, see e.g., Table 3)

| | | | | | (g) GSK | |
|---|---|---|---|---|---|---|
| (b) at | | | | | inhibitor | |
| least one | | | | | and/or Wnt | (h) TGF-β |
| fibroblast | | (d) extracellular | (e) basement | (f) growth | and β- | type I |
| growth | | matrix | membrane | factor for | catenin | receptor |
| factor | (c) neuregulin | glycoprotein | matrix | epithelium | activator | inhibitor |
| X | | | | | | |
| | X | | | | | |
| X | X | | | | | |
| | | X | | | | |
| X | | X | | | | |
| | X | X | | | | |
| X | X | X | | | | |
| | | | X | | | |
| X | | | X | | | |
| | X | | X | | | |
| X | X | | X | | | |
| | | X | X | | | |
| X | | X | X | | | |
| | X | X | X | | | |
| X | X | X | X | | | |
| | | | | X | | |
| X | | | | X | | |
| | X | | | X | | |
| X | X | | | X | | |
| | | X | | X | | |
| X | | X | | X | | |
| | X | X | | X | | |
| X | X | X | | X | | |
| | | | X | X | | |
| X | | | X | X | | |
| | X | | X | X | | |
| X | X | | X | X | | |
| | | X | X | X | | |
| X | | X | X | X | | |
| | X | X | X | X | | |
| X | X | X | X | X | | |
| | | | | | X | |
| X | | | | | X | |
| | X | | | | X | |
| X | X | | | | X | |
| | | X | | | X | |
| X | | X | | | X | |
| | X | X | | | X | |
| X | X | X | | | X | |
| | | | X | | X | |
| X | | | X | | X | |
| | X | | X | | X | |
| X | X | | X | | X | |
| | | X | X | | X | |
| X | | X | X | | X | |
| | X | X | X | | X | |
| X | X | X | X | | X | |
| | | | | X | X | |
| X | | | | X | X | |
| | X | | | X | X | |
| X | X | | | X | X | |
| | | X | | X | X | |
| X | | X | | X | X | |
| | X | X | | X | X | |
| X | X | X | | X | X | |
| | | | X | X | X | |
| X | | | X | X | X | |
| | X | | X | X | X | |
| X | X | | X | X | X | |
| | | X | X | X | X | |
| X | | X | X | X | X | |
| | X | X | X | X | X | |
| X | X | X | X | X | X | |
| | | | | | | X |
| X | | | | | | X |
| | X | | | | | X |
| X | X | | | | | X |
| | | X | | | | X |
| X | | X | | | | X |
| | X | X | | | | X |
| X | X | X | | | | X |
| | | | X | | | X |
| X | | | X | | | X |
| | X | | X | | | X |
| X | X | | X | | | X |
| | | X | X | | | X |
| X | | X | X | | | X |
| | X | X | X | | | X |
| X | X | X | X | | | X |
| | | | | X | | X |
| X | | | | X | | X |
| | X | | | X | | X |
| X | X | | | X | | X |
| | | X | | X | | X |
| X | | X | | X | | X |
| | X | X | | X | | X |
| X | X | X | | X | | X |
| | | | X | X | | X |
| X | | | X | X | | X |
| | X | | X | X | | X |
| X | X | | X | X | | X |
| | | X | X | X | | X |
| X | | X | X | X | | X |
| | X | X | X | X | | X |
| X | X | X | X | X | | X |
| | | | | | X | X |
| X | | | | | X | X |
| | X | | | | X | X |
| X | X | | | | X | X |
| | | X | | | X | X |
| X | | X | | | X | X |
| | X | X | | | X | X |
| X | X | X | | | X | X |
| | | | X | | X | X |
| X | | | X | | X | X |
| | X | | X | | X | X |
| X | X | | X | | X | X |
| | | X | X | | X | X |
| X | | X | X | | X | X |
| | X | X | X | | X | X |
| X | X | X | X | | X | X |
| | | | | X | X | X |
| X | | | | X | X | X |
| | X | | | X | X | X |
| X | X | | | X | X | X |
| | | X | | X | X | X |
| X | | X | | X | X | X |
| | X | X | | X | X | X |
| X | X | X | | X | X | X |
| | | | X | X | X | X |
| X | | | X | X | X | X |
| | X | | X | X | X | X |
| X | X | | X | X | X | X |
| | | X | X | X | X | X |
| X | | X | X | X | X | X |
| | X | X | X | X | X | X |
| X | X | X | X | X | X | X |

In some embodiments, the medium supplement comprises: (i) transferrin; (ii) insulin; (iii) progesterone; (iv) putrescine: (v) selenite: or any combination thereof. Table 3 provides non-limiting examples of combinations of components in the medium supplement. The concentrations of the components of the medium supplement in the salivary gland organoid medium can be the same or different as those described in the medium supplement in the salivary gland organoid differentiation media.

### Uses of Salivary Gland Organoid Cultures

In multiple aspects, described herein are uses of the salivary gland organoid described herein as a model for a salivary gland-associated disease. In other aspects, described herein are uses of the salivary gland organoid culture as described herein to screen for an effective therapeutic for a salivary gland-associated disease.

In one aspect, described herein is a method of screening for an effective therapeutic for a salivary gland-associated disease. In some embodiments, the method comprises: (a) exposing a salivary gland organoid culture as described herein to a potential therapeutic for a salivary gland-associated disease; and (b) determining whether the salivary gland organoid culture exhibits decreased levels of a disease-associated marker for the salivary gland-associated disease or increased levels of a health-associated marker compared to the salivary gland organoid culture not exposed to the potential therapeutic. In some embodiments, the method comprises: (c) determining that the potential therapeutic is an effective therapeutic for the salivary gland-associated disease if the salivary gland organoid culture exhibits decreased levels of a disease-associated marker for the salivary gland-associated disease or increased levels of a health-associated marker compared to the salivary gland organoid culture not exposed to the potential therapeutic. In some embodiments, the method comprises: (d) determining that the potential therapeutic is not an effective therapeutic for the salivary gland-associated disease if the salivary gland organoid culture did not exhibit decreased levels of a disease-associated marker for the salivary gland-associated disease or increased levels of a health-associated marker compared to the salivary gland organoid culture not exposed to the potential therapeutic. In some embodiments, the health-associated marker is selected from the group consisting of: Amylase Alpha 1A (AMY1A), keratin 19 (KRT19), and Actin Alpha 2 (ACTA2).

In some embodiments, the salivary gland-associated disease is a metabolic disease. In some embodiments, the metabolic disease is type 2 diabetes. In some embodiments, the salivary gland-associated disease is an inflammatory disease. In some embodiments, the inflammatory disease is Sjögren's syndrome. In some embodiments, the salivary gland-associated disease is an autoimmune disease. In some embodiments, autoimmune disease is type 1 diabetes. In some embodiments, the salivary gland-associated disease is radiation damage. In some embodiments, the radiation damage is from radiation treatment for a cancer, such as a head or neck cancer.

In some embodiments of any one of the methods described, the autoimmune disease or disorder is selected from the group consisting of thyroiditis, type 1 diabetes mellitus, Hashimoto's thyroidits, Graves' disease, multiple sclerosis, Guillain-Barre syndrome, Addison's disease, and Raynaud's phenomenon, Goodpasture's disease, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, guttate psoriasis, pustular psoriasis, and psoriasis of the nails, atopy including atopic diseases such as hay fever and Job's syndrome, dennatitis including contact dennatitis, chronic contact dermatitis, exfoliative dennatitis, allergic dermatitis, allergic contact dermatitis, dennatitis herpetifonnis, nummular dermatitis, seborrheic dennatitis, non-specific dermatitis, primary irritant contact dermatitis, and atopic dermatitis, x-linked hyper IgM syndrome. allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidennal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyodenna gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritus scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, erythema multiforme, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B—O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, diabetic large-artery disorder, immune responses associated with acute and delayed hypersensitivity mediated by cytokines and T-lymphocytes, sarcoidosis, granulomatosis including lymphomatoid granulomatosis. Wegener's granulomatosis, agranulocytosis, vasculitides, including vasculitis, large-vessel vasculitis (including polymyalgia rheumatica and giant-cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis. and ANCA-associated vasculitis, such as Churg-Strauss vasculitis or syndrome (CSS) and ANCA-associated small-vessel vasculitis, temporal arteritis, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis. CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome. allergic neuritis. Behcet's disease/syndrome, Castleman's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, an immune complex disorder such as immune complex nephritis, antibody-mediated nephritis, polyneuropathies, chronic neuropathy such as IgM polyneuropathies or IgM-mediated neuropathy, and autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, idiopathic hypothyroidism, polyglandular syndromes such as autoimmune polyglandular syndromes (or polvglandular endocrinopathy syndromes), parancoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE). myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, lupoid hepatitis, giant-cell hepatitis, autoimmune chronic active hepatitis, lymphoid interstitial pneumonitis (LIP), bronchiolitis obliterans (non-transplant) vs NSIP. Berger's disease (IgA nephropathy), idiopathic IgA nephropathy. linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amyotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g.. benign monoclonal gammopathy and monoclonal gammopathy of undetermined significance, MGUS), peripheral neuropathy, paraneoplastic syndrome, channelopathies including channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder. fibromyalgia, multiple endocrine failure, Schmidt's syndrome, adrenalitis, gastric atrophy, presenile dementia. demyelinating diseases such as autoimmune demyelinating diseases and chronic inflammatory demyelinating polyneuropathy. Dressler's syndrome, alopecia areata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, Sampter's syndrome, Caplan's syndrome, endocarditis, endomyocardial fibrosis, diffuse interstitial pulmonary fibrosis, interstitial hmg fibrosis, pulmonary fibrosis, idiopathic pulmonary fibrosis, cystic fibrosis, endophthalmitis, erythema elevatum et diutinum, erythroblastosis fetalis, eosinophilic fasciitis, Shulman's syndrome, Felty's syndrome, cyclitis such as chronic cyclitis, heterochromic cyclitis, iridocyclitis (acute or chronic), or Fuch's cyclitis, Henoch-Schonlein purpura, SCID, sepsis, endotoxemia, post-vaccination syndromes, Evan's syndrome, autoimmune gonadal failure, Sydenham's chorea, post-streptococcal nephritis, thromboangiitis obliterans, thyrotoxicosis, tabes dorsalis, chorioiditis, giant-cell polymyalgia, chronic hypersensitivity pneumonitis, keratoconjunctivitis sicca, idiopathic nephritic syndrome, minimal change nephropathy, benign familial and ischemia-reperfusion injury, transplant organ reperfusion, retinal autoimmunity, aphthae, aphthous stomatitis, arteriosclerotic disorders, aspermiogenesis, autoimmune hemolysis. Boeck's disease, enteritis allergica, erythema nodosum leprosum, idiopathic facial paralysis, chronic fatigue syndrome, febris rheumatica. Hamman-Rich's disease, sensoneural hearing loss, ileitis regionalis, leucopenia, transverse myelitis, primary idiopathic myxedema, ophthalmia symphatica, polyradiculitis acuta, pyoderma gangrenosum, acquired splenic atrophy, vitiligo, toxic-shock syndrome, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, rheumatic diseases, mixed connective tissue disease, nephrotic syndrome, insulitis, polyendocrine failure, autoimmune polyglandular syndrome type I, adult-onset idiopathic hypoparathyroidism (AOIH), myocarditis, nephrotic syndrome, primary sclerosing cholangitis. acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, granulomas containing eosinophils, seronegative spondyloarthritides, polyendocrine autoimmune disease, sclerosing cholangitis, sclera, episclera, Bruton's syndrome, transient hypogammaglobulinemia of infancy. Wiskott-Aldrich syndrome, ataxia telangiectasia syndrome, angiectasis, autoimmune disorders associated with collagen disease, rheumatism, allergic hypersensitivity disorders. glomerulonephritides, reperfusion injury, ischemic re-perfusion disorder, lymphomatous tracheobronchitis, inflammatory dermatoses, dermatoses with acute inflammatory components, and autoimmune uveoretinitis (AUR).

### DEFINITIONS

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all techmical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

The terms “decrease”, “reduced”, “reduction”, or “inhibit” are all used herein to mean a decrease by a statistically significant amount. In some embodiments. “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g.. Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a salivary gland-associated disease. A subject can be male or female.

A subject can be one who has been previously diagnosed with or identified as suffering from or having a condition in need of treatment (e.g. salivary gland-associated disease or one or more complications related to such a condition, and optionally, have already undergone treatment for a salivary gland-associated disease or the one or more complications related to a salivary gland-associated disease. Alternatively, a subject can also be one who has not been previously diagnosed as having a salivary gland-associated disease or one or more complications related to a salivary gland-associated disease. For example, a subject can be one who exhibits one or more risk factors for a salivary gland-associated disease or one or more complications related to a salivary gland-associated disease or a subject who does not exhibit risk factors.

A “subject in need” of treatment for a particular condition can be a subject having that condition, diagnosed as having that condition, or at risk of developing that condition.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (e.g., mRNA) or antisense RNA derived from a nucleic acid fragment or fragments and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” refers to the nucleic acid sequence which is transcribed (DNA) to RNA in vitro or in vivo when operably linked to appropriate regulatory sequences. The gene may or may not include regions preceding and following a coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g.. nucleic acid or polypeptide which is differentially present in a sample taken from subjects having a salivary gland-associated disease, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g., a salivary gland-associated disease, e.g., a metabolic disease, an inflammatory disease, an autoimmune disease, and/or radiation damage. The term “treating” includes reducing or alleviating at least one adverse effect or symptom of a condition, disease or disorder associated with a salivary gland-associated disease. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

In some embodiments of any of the aspects, the cells can be maintained in culture. As used herein, “maintaining” refers to continuing the viability of a cell or population of cells. A maintained population of cells will have at least a subpopulation of metabolically active cells.

As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean 1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company. 2016 (ISBN 0815345054, 978-0815345053): Lewin's Genes XI. published by Jones & Bartlett Publishers. 2014 (ISBN-1449659055); Michael Richard Green and Joseph Sambrook, Molecular Cloning: A Laboratory Manual, 4th ed.. Cold Spring Harbor Laboratory Press, Cold Spring Harbor, N.Y., USA (2012) (ISBN 1936113414); Davis et al., Basic Methods in Molecular Biology. Elsevier Science Publishing, Inc., New York, USA (2012) (ISBN 044460149X); Laboratory Methods in Enzymology: DNA, Jon Lorsch (ed.) Elsevier, 2013 (ISBN 0124199542): Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (Join E. Coligan, ADA M Kruisbeek, David H Margulies, Ethan M Shevach, Warren Strobe, (eds.) Join Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references. issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore. while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A composition comprising a salivary gland organoid culture.
2. The composition of paragraph 1, wherein the salivary gland organoid culture comprises salivary gland epithelial cells.
3. The composition of paragraph 2, wherein the salivary gland epithelial cells express acinar marker Amylase Alpha 1A (AMY1A), duct marker keratin 19 (KRT19), myoepithelial marker Actin Alpha 2 (ACTA2), and/or neuroepithelium marker Tubulin Beta 3 Class III (TUBB3).
4. The composition of any one of paragraphs 1-3, wherein the salivary gland organoid culture comprises salivary gland buds, branches, and/or lumenized ducts.
5. The composition of any one of paragraphs 1-4, wherein the salivary gland organoid culture is substantially free of endothelium and mesenchyme.
6. The composition of any one of paragraphs 1-5, wherein the salivary gland organoid culture is derived from pluripotent stem cells.
7. The composition of any one of paragraphs 1-6, wherein the salivary gland organoid culture can produce saliva.
8. The composition of any one of paragraphs 1-7, wherein the composition further comprises a salivary gland organoid culture medium.
9. The composition of paragraph 8, wherein the salivary gland organoid medium comprises:
   a) a medium supplement comprising:
      i) transferrin;
      ii) insulin;
      iii) progesterone;
      iv) putrescine; and/or
      v) selenite;
   b) at least one fibroblast growth factor;
   c) a neuregulin;
   d) an extracellular matrix glycoprotein;
   e) a basement membrane matrix;
   f) a growth factor for epithelium;
   g) an inhibitor of a Glycogen Synthase Kinase (GSK) and/or activator of a Wingless/Integrated (Wnt) and β-catenin pathway; and/or
   h) an inhibitor of transforming growth factor-β (TGF-β) type I receptor.
10. A method of establishing a salivary gland organoid culture, the method comprising:
   a) culturing a population of pluripotent stem cells in aggregation medium for a sufficient time to promote formation of pluripotent stem cell clusters;
   b) culturing the resultant pluripotent stem cell clusters in a salivary epithelium differentiation medium for a sufficient time to promote differentiation into salivary epithelium; and
   c) culturing the resultant salivary epithelium in a salivary gland organoid differentiation medium for a sufficient time to promote differentiation into a salivary gland organoid culture.
11. The method of paragraph 10, wherein the pluripotent stem cells comprise induced pluripotent stem cells (iPSCs).
12. The method of paragraph 11. wherein the iPSCs are derived from a somatic cell sample from a subject.
13. The method of paragraph 11 or paragraph 12, wherein the iPS cell is derived from a subject with a disease or disorder that affects salivary gland function.
14. The method of any one of paragraphs 11-13, wherein the iPSCs comprise cell line WTC-11.
15. The method of any one of paragraphs 10-14, wherein the aggregation medium comprises an inhibitor of Rho Associated Coiled-Coil Containing Protein Kinase (ROCK).
16. The method of paragraph 15, wherein the inhibitor of ROCK is Y-27632.
17. The method of paragraph 15 or 16, wherein the inhibitor of ROCK is at a concentration of about 50 μM.
18. The method of any one of paragraphs 10-17, wherein the aggregation medium comprises mTeSR™1 medium.
19. The method of any one of paragraphs 10-18, wherein the sufficient amount of time to promote formation of pluripotent stem cell clusters is about 3 days.
20. The method of any one of paragraphs 10-19, wherein the salivary epithelium differentiation medium comprises at least one of the following:
   a) an agonist of Smoothened from about day 0 to about day 8 after beginning the salivary epithelium differentiation medium;
   b) a bone morphogenetic protein from about day 3 to about day 8 after beginning the salivary epithelium differentiation medium;
   c) from about day 8 to about day 12 after beginning the salivary epithelium differentiation medium:
      i) an agonist of Smoothened;
      ii) an inhibitor of a Glycogen Synthase Kinase (GSK) and/or activator of a Wingless/Integrated (Wnt) and β-catenin pathway:
      iii) a growth factor for epithelium;
      iv) an inhibitor of a Bone Morphogenetic Protein type I (BMP I) receptor; and/or
      v) a neurotrophin; and/or
   d) a fibroblast growth factor from about day 10 to about day 12 after beginning the salivary epithelium differentiation medium.
21. The method of any one of paragraphs 10-20, wherein the salivary epithelium differentiation medium comprises at least one of the following:
   a) an agonist of Smoothened at a concentration of about 400 nM from about day 0 to about day 8 after beginning the salivary epithelium differentiation medium;
   b) a bone morphogenetic protein at a concentration of about 150 pM from about day 3 to about day 8 after beginning the salivary epithelium differentiation medium;
   c) from about day 8 to about day 12 after beginning the salivary epithelium differentiation medium:
      i) an agonist of Smoothened at a concentration of about 1 mM:
      ii) an inhibitor of a Glycogen Synthase Kinase (GSK) and/or activator of a Wingless/Integrated (Wnt) and β-catenin pathway at a concentration of about 5 μM;
      iii) a growth factor for epithelium at a concentration of about 15 ng/mL;
      iv) an inhibitor of a Bone Morphogenetic Protein type I (BMP I) receptor at a concentration of about 1 μM; and/or
      v) a neurotrophin at a concentration of about 1 μM; and/or
   d) a fibroblast growth factor at a concentration of about 250 ng/mL from about day 10 to about day 12 after beginning the salivary epithelium differentiation medium.
22. The method of paragraph 20 or 21, wherein:
   a) the agonist of Smoothened is Smoothened Agonist (SAG);
   b) the bone morphogenetic protein is bone morphogenetic protein 4 (BMP4);
   c) the inhibitor of GSK and/or activator of the Wnt and β-catenin pathway is Chiron (Chir99021);
   d) the growth factor for epithelium is epidermal growth factor (EGF);
   e) the inhibitor of the BMP type I receptor is LDN-193189;
   f) the neurotrophin is Neurotrophin-4; and/or
   g) the fibroblast growth factor is fibroblast growth factor 10 (FGF10).
23. The method of any one of paragraphs 10-22, wherein the salivary epithelium differentiation medium comprises at least one of the following:
   a) Smoothened Agonist (SAG) at a concentration of about 400 nM from about day 0 to about day 8 after beginning the salivary epithelium differentiation medium;
   b) bone morphogenetic protein 4 (BMP4) at a concentration of about 150 pM from about day 3 to about day 8 after beginning the salivary epithelium differentiation medium;
   c) from about day 8 to about day 12 after beginning the salivary epithelium differentiation medium:
      i) SAG at a concentration of about 1 mM;
      ii) Chiron (Chir99021) at a concentration of about 5 μM;
      iii) epidennal growth factor (EGF) at a concentration of about 15 ng/mL;
      iv) an inhibitor of a Bone Morphogenetic Protein type I receptor (BMP I) LDN-193189 at a concentration of about 1 μM; and/or
      v) Neurotrophin-4 at a concentration of about 1 μM; and/or
   d) fibroblast growth factor 10 (FGF10) at a concentration of about 250 ng/mL from about day 10 to about day 12 after beginning the salivary epithelium differentiation medium.
24. The method of any one of paragraphs 10-23. wherein the salivary epithelium differentiation medium comprises EPICULT™-C medium.
25. The method of any one of paragraphs 10-24, wherein the sufficient amount of time to promote differentiation of pluripotent stem cell clusters into salivary epithelium is about 12 days.
26. The method of any one of paragraphs 10-25, wherein the salivary gland organoid differentiation medium comprises at least one of the following:
   a) from about day 12 to about day 35-75 after beginning the salivary epithelium differentiation medium (or from about day 0 to about day 23-63 after beginning the salivary gland organoid differentiation medium):
      i) a medium supplement comprising:
         A) transferrin;
         B) insulin;
         C) progesterone;
         D) putrescine; and/or
         E) selenite; and/or
      ii) at least one fibroblast growth factor;
   b) a neuregulin from about day 18 to about day 35-75 after beginning the salivary epithelium differentiation medium (or from about day 6 to about day 23-63 after beginning the salivary gland organoid differentiation medium);
   c) from about day 18 to about day 24-75 after beginning the salivary epithelium differentiation medium (or from about day 6 to about day 12-63 after beginning the salivary gland organoid differentiation medium):
      i) an extracellular matrix glycoprotein; and/or
      ii) a basement membrane matrix:
   d) an inhibitor of Rho Associated Coiled-Coil Containing Protein Kinase (ROCK) from about day 24 to about day 27 after beginning the salivary epithelium differentiation medium (or from about day 12 to about day 15 after beginning the salivary gland organoid differentiation medium); and/or
   e) from about day 24 to about day 35-75 after beginning the salivary epithelium differentiation medium (or from about day 12 to about day 23-63 after beginning the salivary gland organoid differentiation medium):
      i) a growth factor for epithelium;
      ii) an inhibitor of a Glycogen Synthase Kinase (GSK) and/or activator of a Wingless/Integrated (Wnt) and β-catenin pathway; and/or
      iii) an inhibitor of transforming growth factor-β (TGF-β) type I receptor.
27. The method of any one of paragraphs 10-26, wherein the salivary gland organoid differentiation medium comprises at least one of the following:
   a) from about day 12 to about day 35-75 after beginning the salivary epithelium differentiation medium (or from about day 0 to about day 23-63 after beginning the salivary gland organoid differentiation medium):
      i) a medium supplement comprising:
         A) transferrin at a concentration of about 10 mg/L;
         B) insulin at a concentration of about 5 mg/L;
         C) progesterone at a concentration of about 6.3 μg/L;
         D) putrescine at a concentration of about 16.11 mg/L; and/or
         E) selenite at a concentration of about 5.2 μg/L; and/or
      ii) at least one fibroblast growth factor at a concentration from about 125 ng/mL to about 250 ng/mL;
   b) a neuregulin at a concentration of about 250 ng/mL from about day 18 to about day 35-75 after beginning the salivary epithelium differentiation medium (or from about day 6 to about day 23-63 after beginning the salivary gland organoid differentiation medium);
   c) from about day 18 to about day 24-75 after beginning the salivary epithelium differentiation medium (or from about day 6 to about day 12-63 after beginning the salivary gland organoid differentiation medium):
      i) an extracellular matrix glycoprotein at a concentration of about 1%; and/or
      ii) a basement membrane matrix at a concentration of about 3%;
   d) an inhibitor of Rho Associated Coiled-Coil Containing Protein Kinase (ROCK) at a concentration of about 50 μM from about day 24 to about day 27 after beginning the salivary epithelium differentiation medium (or from about day 12 to about day 15 after beginning the salivary gland organoid differentiation medium); and/or
   e) from about day 24 to about day 35-75 after beginning the salivary epithelium differentiation medium (or from about day 12 to about day 23-63 after beginning the salivary gland organoid differentiation medium):
      i) a growth factor for epithelium at a concentration of about 100 ng/mL;
      ii) an inhibitor of a Glycogen Synthase Kinase (GSK) and/or activator of a Wingless/Integrated (Wnt) and β-catenin pathway at a concentration of about 5 μM; and/or
      iii) an inhibitor of transforming growth factor-Q (TGF-3) type I receptor at a concentration of about 10 μM.
28. The method of paragraph 26 or 27, wherein:
   a) the media supplement is N-2 supplement;
   b) the neuregulin is Neuregulin 1 (NRG1);
   c) the at least one fibroblast growth factor is selected from the group consisting of: fibroblast growth factor 10 (FGF10), fibroblast growth factor 7 (FGF7), and fibroblast growth factor 1 (FGF1);
   d) the extracellular matrix glycoprotein is a laminin;
   e) the basement membrane matrix is secreted by Engelbreth-Holm-Swarm (EHS) mouse sarcoma cells (MATRIGEL®);
   f) the inhibitor of ROCK is Y-27632;
   g) the growth factor for epithelium is epidermal growth factor (EGF);
   h) the inhibitor of GSK and/or activator of the Wnt and β-catenin pathway is Chiron (Chir99021); and/or
   i) the inhibitor of the TGF-β type I receptor is SB-431452.
29. The method of paragraph 27 or 28, wherein the basement membrane matrix is growth factor reduced (GFR).
30. The method of any one of paragraphs 10-29, wherein the salivary gland organoid differentiation medium comprises at least one of the following:
   a) from about day 12 to about day 35-75 after beginning the salivary epithelium differentiation medium (or from about day 0 to about day 23-63 after beginning the salivary gland organoid differentiation medium):
      i) an N-2 medium supplement comprising:
         A) transferrin at a concentration of about 10 mg/L;
         B) insulin at a concentration of about 5 mg/L;
         C) progesterone at a concentration of about 6.3 μg/L;
         D) putrescine at a concentration of about 16.11 mg/L; and/or
         E) selenite at a concentration of about 5.2 μg/L;
      ii) fibroblast growth factor 10 (FGF10) at a concentration of about 250 ng/mL;
      iii) fibroblast growth factor 7 (FGF7) at a concentration of about 125 ng/mL; and/or
      iv) fibroblast growth factor 1 (FGF1) at a concentration of about 125 ng/mL;
   b) NRG1 at a concentration of about 250 ng/mL from about day 18 to about day 35-75 after beginning the salivary epithelium differentiation medium (or from about day 6 to about day 23-63 after beginning the salivary gland organoid differentiation medium);
   c) from about day 18 to about day 24-75 after beginning the salivary epithelium differentiation medium (or from about day 6 to about day 12-63 after beginning the salivary gland organoid differentiation medium):
      i) laminin at a concentration of about 1%; and/or
      ii) MATRIGEL® at a concentration of about 3%;
   d) Y-27632 at a concentration of about 50 μM from about day 24 to about day 27 after beginning the salivary epithelium differentiation medium (or from about day 12 to about day 15 after beginning the salivary gland organoid differentiation medium); and/or
   e) from about day 24 to about day 35-75 after beginning the salivary epithelium differentiation medium (or from about day 12 to about day 23-63 after beginning the salivary gland organoid differentiation medium):
      i) EGF at a concentration of about 100 ng/mL;
      ii) Chiron at a concentration of about 5 μM; and/or
      iii) SB-431452 at a concentration of about 10 μM.
31. The method of any one of paragraphs 10-30, wherein the salivary gland organoid differentiation medium comprises Dulbecco's Modified Eagle Medium and Nutrient Mixture F12 (DMEM/F12™) medium.
32. The method of any one of paragraphs 10-31, wherein the sufficient amount of time to promote differentiation of salivary epithelium into the salivary gland organoid culture is about 23-63 days.
33. The method of any one of paragraphs 10-32, wherein the sufficient amount of time to promote differentiation of salivary epithelium into the salivary gland organoid culture is about 38 days.
34. The method of any one of paragraphs 10-33, wherein the sufficient amount of time to promote differentiation of the population of pluripotent stem cells into the salivary gland organoid culture is about 53 days.
35. The method of any one of paragraphs 10-34, wherein the sufficient amount of time to promote differentiation of the pluripotent stem cell clusters into the salivary gland organoid culture is about 50 days.
36. The method of any one of paragraphs 10-35, wherein the sufficient amount of time to promote differentiation of the pluripotent stem cell clusters into the salivary gland organoid culture is about 30 days to 50 days.
37. The method of any one of paragraphs 10-36, wherein the salivary gland organoid culture secretes salivary amylase after about day 35 after beginning the salivary epithelium differentiation medium.
38. The method of any one of paragraphs 10-37, wherein the salivary gland organoid differentiation medium further comprises from at least about day 35 after beginning the salivary epithelium differentiation medium (or from at least about day 27 after beginning the salivary gland organoid differentiation medium):
   a) a sugar or sugar alcohol; and/or
   b) at least one proinflammatory cytokine.
39. The method of any one of paragraphs 10-38, wherein the salivary gland organoid differentiation medium further comprises from at least about day 35 after beginning the salivary epithelium differentiation medium (or from at least about day 27 after beginning the salivary gland organoid differentiation medium):
   a) a sugar or sugar alcohol at a concentration of about 53-70 mM; and/or
   b) at least one proinflammatory cytokine at a concentration of about 1 ng/ml.
40. The method of paragraph 38 or 39, wherein:
   a) the sugar is D-glucose;
   b) the sugar alcohol is D-mannitol; and/or
   c) the at least one proinflammatory cytokine is TNFa and/or IL-6.
41. The method of any one of paragraphs 10-40, wherein the salivary gland organoid differentiation medium further comprises from at least about day 35 after beginning the salivary epithelium differentiation medium (or from at least about day 27 after beginning the salivary gland organoid differentiation medium):
   a) D-glucose or D-mannitol at a concentration of about 53-70 mM; and/or
   b) TNFa and/or IL-6 at a concentration of about 1 ng/ml.
42. A salivary gland organoid culture prepared according to the method of any one of paragraphs 10-41.
43. A salivary gland organoid culture derived from pluripotent stem cells.
44. A salivary gland organoid culture of any of paragraphs 10-41, implanted into the kidney capsule of a subject.
45. Use of the salivary gland organoid culture of any one of paragraphs 1-9 or 42-44 as a model for a salivary gland-associated disease.
46. Use of the salivary gland organoid culture of any one of paragraphs 1-9 or 42-44 to screen for an effective therapeutic for a salivary gland-associated disease.
47. The use of paragraph 45 or 46, wherein the salivary gland-associated disease is selected from the group consisting of
   a) a metabolic disease;
   b) an inflammatory disease;
   c) an autoimmune disease; and/or
   d) radiation damage.
48. The use of paragraph 47, wherein:
   a) the metabolic disease is type 2 diabetes;
   b) the inflammatory disease is Sjögren's syndrome;
   c) the autoimmune disease is type 1 diabetes; and/or
   d) the radiation damage is from radiation treatment for head or neck cancer.
49. The use of any one of paragraphs 45-48, wherein the salivary gland organoid culture is implanted into the kidney capsule of a subject.
50. A method of screening for an effective therapeutic for a salivary gland-associated disease, the method comprising:
   a) exposing the salivary gland organoid culture of any one of paragraphs 1-9 or 42-44 to a potential therapeutic for a salivary gland-associated disease;
   b) determining whether the salivary gland organoid culture exhibits decreased levels of a disease-associated marker for the salivary gland-associated disease or increased levels of a health-associated marker compared to the salivary gland organoid culture not exposed to the potential therapeutic; and
   c) determining that the potential therapeutic is an effective therapeutic for the salivary gland-associated disease if the salivary gland organoid culture exhibits decreased levels of a disease-associated marker for the salivary gland-associated disease or increased levels of a health-associated marker compared to the salivary gland organoid culture not exposed to the potential therapeutic: or
   d) determining that the potential therapeutic is not an effective therapeutic for the salivary gland-associated disease if the salivary gland organoid culture did not exhibit decreased levels of a disease-associated marker for the salivary gland-associated disease or increased levels of a health-associated marker compared to the salivary gland organoid culture not exposed to the potential therapeutic.
51. The method of paragraph 50, wherein the health-associated marker is selected from the group consisting of: Amylase Alpha 1A (AMY1A), keratin 19 (KRT19), and Actin Alpha 2 (ACTA2).

The technology described herein is further illustrated by the following examples which in no way should be construed as being further limiting.

### EXAMPLES

### Example 1: Rapid Sci-Seq-Guided iPSC-Derived Salivary Gland Organoids

### 1. Introduction:

Exocrine glands, including mammary, prostate, sweat, lacrimal, and salivary, are epithelial tissues comprised of duct systems through which they secrete various factors onto adjacent surfaces. Epithelial carcinomas account for majority of the cancer cases (90%). However, identifying the tissue origin of the carcinoma is hampered by the fact that while the cancer cells are reverting to a fetal stage, human fetal development of many exocrine glands is still poorly understood.

General exocrine gland development shares a complex stepwise process including epithelial ingrowth to form a bud, branching morphogenesis and ductal elongation, and further, secretory cell differentiation. It has been well established that epithelial-mesenchymal interactions, integrin-, and FGF10-signaling are involved in exocrine gland morphogenesis and development. However, the mechanism of action of signaling pathways, as well as specific transcription factors that drive cell fate decisions during different exocrine gland development have remained largely elusive. Increased accessibility to single cell sequencing technologies can remedy this.

The salivary gland is an exocrine gland that produces and secretes saliva. In humans, there are three major types of glands, the parotid, the submandibular, and the sublingual glands, which produce approximately 90% of all saliva, and several minor salivary glands dispersed throughout the oral submucosa make up the other 10 percent. Saliva plays roles, including but not limited to tissue repair, oral lubrication, tooth mineralization and protection, and taste. Several factors can perturb the proper function of salivary glands, including Sjögren's syndrome, as well as cancer and resultant radiation therapy. Compounding this issue is that salivary glands have been shown to have poor regenerative capacity following injury despite reports of the existence of stem-like cells in adult tissues.

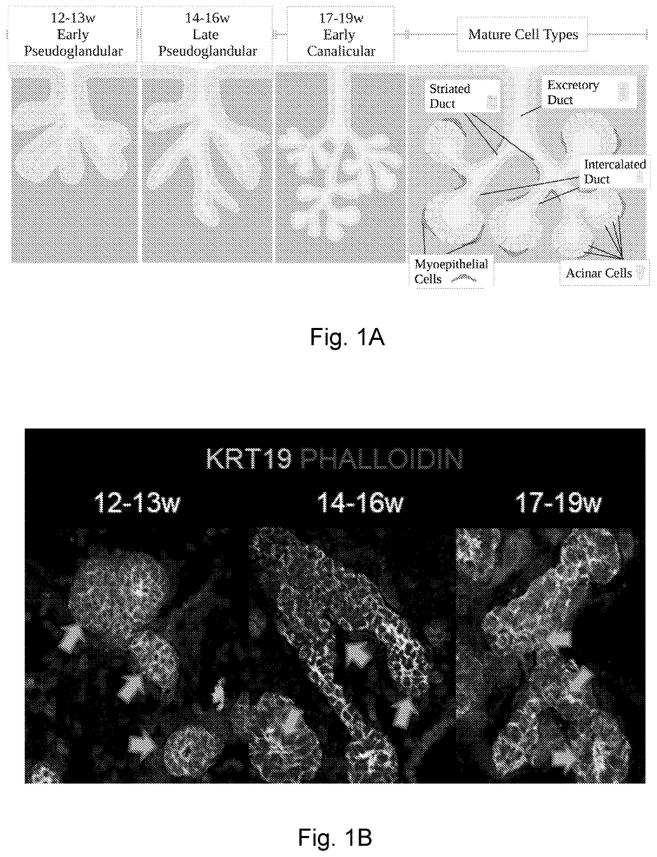

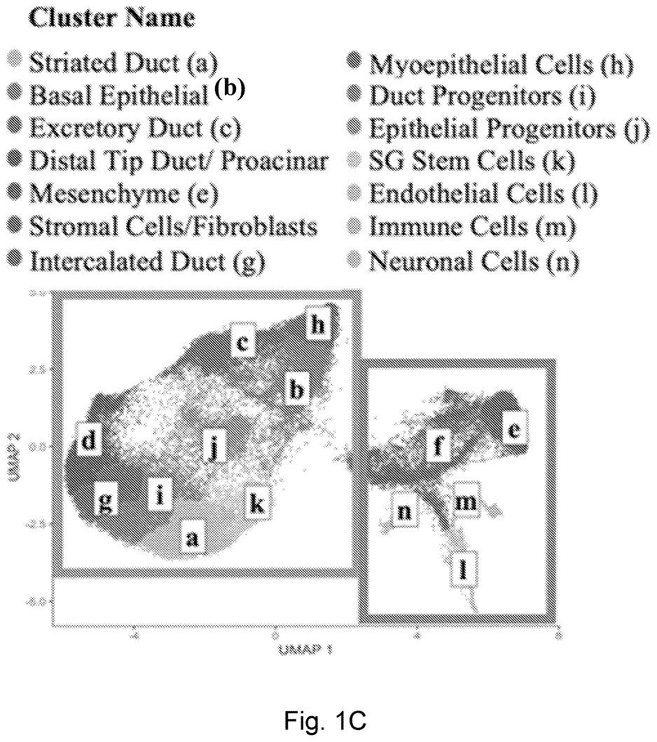

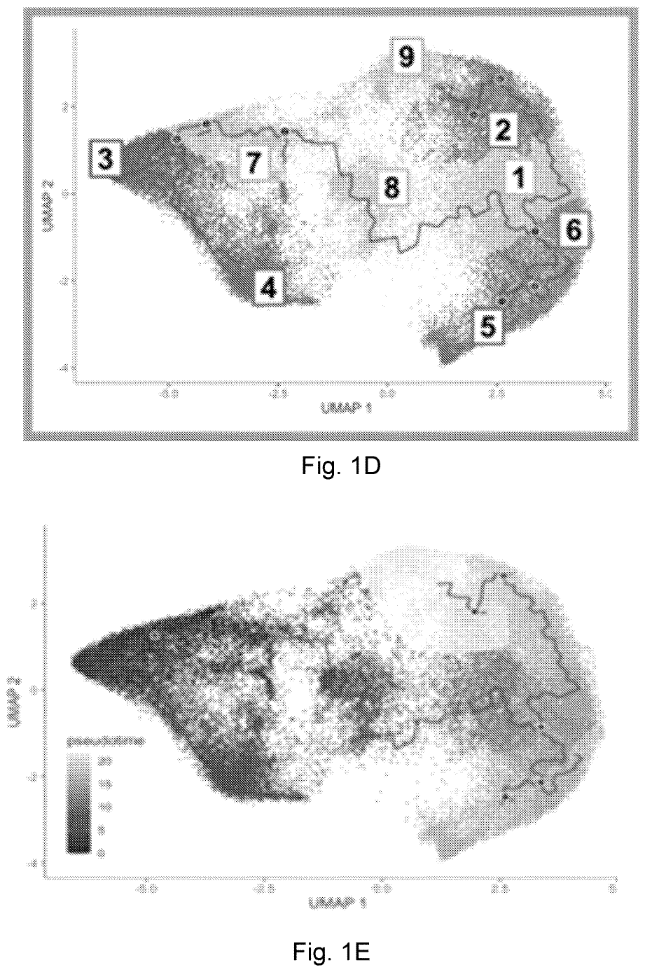

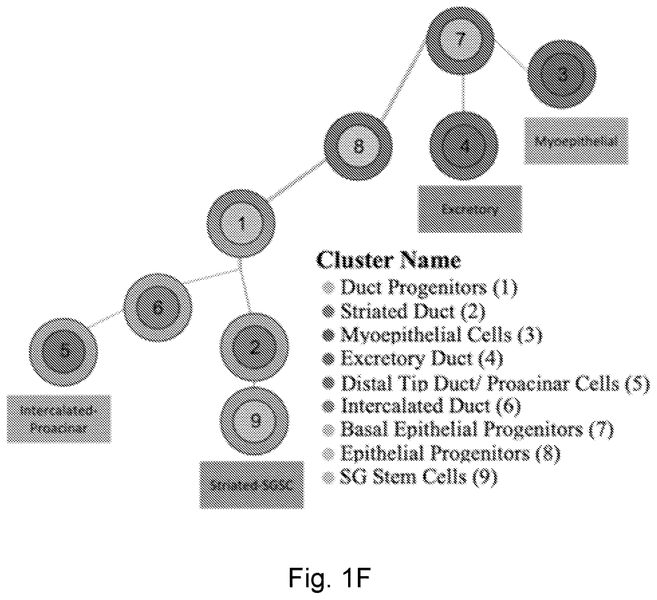

Salivary gland development begins in humans between 6-8 weeks gestation and continues developing after birth. Because of the early stage at which salivary glands develop in humans, the overwhelming majority of what is currently known about salivary gland development has been learned through molecular, cytological, and morphological studies using murine models. In mice, salivary gland development begins with the invagination of the thickened epithelium into the underlying condensed mesenchyme to yield the initial bud stage (E12.5). Branching morphogenesis and tubulogenesis yield ducts (E13.5-E14.5), and lumenization proceeds through the canalicular stage at E16. Lumenization is a stepwise process whereby lumens form first in the distal end of the main cord and the branch cords, followed by the proximal end of the main cords, and finally the central portion of the main cord. Terminal differentiation results in secretory acini (E17.5) that are regulated by nerve stimulation. The salivary gland is comprised of a network of branched ducts that terminate in saliva-producing acini. The acini produce primary saliva, which is an isotonic solution containing amylases, mucins, and extracellular fluid. They are connected to intercalated ducts (IDs) which receive the primary saliva and serve as a transition between the acini and the functional striated duct (SD), performing minimal ion exchange into the primary saliva. SDs perform a significant amount active transport to drive ion exchange and water reabsorption to yield hypotonic secondary saliva. This is then transported to the excretory duct, which opens into the oral cavity (see e.g., FIG. 1A-1C).

Several major signaling pathways have been identified in salivary gland development in murine models. For example, signaling via FGFR and ERBB3/EGFR are involved in early salivary gland morphogenesis and development. Moreover, crosstalk between FGF and WNT signals have been shown to distinguish between the expanding endbud and the differentiating duct, and that Wnt-related transcription factor Tfcp211 is required for patterning of salivary gland ducts. However, while previous studies have implicated these factors as regulators of early morphogenetic properties like proliferation and branching morphogenesis, thus far, the pathways that regulate individual cell fate choices were largely unknown. Recently, several groups have undertaken single cell sequencing in mouse salivary glands at various ages. These studies underscore the complexity of salivary gland development, highlighting the heterogeneity between glands and identifying factors involved in early development. However, human salivary gland signaling pathways and cell-cell interactions that drive individual lineages and cell fate decisions were still to be dissected. To understand the fate and lineage decisions in human salivary gland development, single cell sequencing was conducted on human fetal salivary tissue and the resulting information was used to develop a 3D hiPSC-derived salivary gland organoid able to self-organize into 3D duct-like structures with lumens that then branch outward, expressing KRT19, which marks duct tissue during human fetal salivary gland development, and acinar marker AMY1A by day 35. The 3D in vitro organoid was further developed as a diabetic model, which showed reduced expression of AMY1A.

### 2. Materials and Methods:

### 2.1 Tissue Collection

This study is approved by an Institutional Review Boards (IRB) for the use of human fetal tissues. Tissue was collected and dissected as described in Alghadeer et al., 2022, BioRxiv, available on the world wide web at biorxiv.org/content/10.1101/2022.08.09.503399v1; Alghadeer et al., 2023, Dev Cell. 58(20): 2163-2180.e9: the contents of each of which are incorporated herein by reference in their entireties. Briefly, tissues from 12-22 weeks gestation were acquired from a Birth Defects Research Laboratory within 6 hours of initial dissection. Salivary glands from 12 weeks gestation and older were isolated from surrounding jaw tissue, and all tissues were snap frozen in liquid nitrogen-cooled 2-Methylbutane and stored at −80 °C until further use. Tissues for sequencing (12-19 weeks) underwent nuclei extraction (see e.g., Alghadeer et al., 2022, Alghadeer et al., 2023). Tissues that were meant for immunofluorescent staining or RNASCOPE were embedded in O.C.T. Compound.

### 2.2 Data Analysis

### 2.2.1 Unbiased Clustering and Cluster Identification

Low quality reads were removed from each sample by excluding cells with a unique molecular identifier (UMI) less than 200 and all cells with a high proportion of mitochondrial UMIs. Quality preprocessed datasets were combined to create a single dataset with all age groups and taken through all subsequent analyses. To prevent obfuscation of cell identity in actively cycling cells. SEURAT was used to regress out common cell cycle-associated genes from consideration for clustering. Cells were clustered using the MONOCLE3 workflow. Briefly, data was normalized by size factor, preprocessed using principal component analysis, dimensions were reduced using the UMAP algorithm, and clustered using unsupervised graph-based clustering analysis via the LEIDEN algorithm. In all computational analyses, the statistical significance is assessed by default calculation of a p-value, a q-value, or a False Discovery Rate set to 0.05 or less; results that fall outside that cutoff are excluded.

Clusters were identified through a combination of literature-derived marker genes from salivary gland and other glandular epithelium and functional characterization of highly expressed genes.

Clusters from the initial analysis were isolated and sub-clustered, following the above procedure with more stringent parameters.

### 2.2.2 Pseudotime Analysis

Pseudotime analysis was performed with the MONOCLE3 default workflow. Briefly, a machine learning technique known as reversed graph embedding is used to “learn” the principal graph and branchpoints that represent the predicted developmental trajectory and embed it back into the graph that represents the single cell dataset (the cluster plot). Cells are then assigned a pseudotime value based on their projection along the predicted trajectory in relation to the root node and plotted in UMAP with coloring indicative of where a given cell falls in the biological process.

### 2.2.3 Differential Expression Analysis, Top Gene Analysis, and ChEA3 Analysis

To identify differentially expressed genes, the R package DE Single was used, which identifies differentially expressed genes between two clusters using a Zero-Inflated Negative Binomial model to faithfully depict significantly expressed genes despite the stochastic nature of transcription at the single cell level. To ensure the program only returned highly significant results. the false discovery rate was set to <0.05 for all analyses.

To identify top expressed genes per cluster, MONOCLE3's top markers function was used. This function classifies genes based on multiple parameters including expression level as well as how unique its expression is to a given cluster and its pseudo R² value and assigns them a “marker score”. The program only considered genes that were expressed in at least 25% of the cells and asked to return the top 300 ranked genes per cluster.

ChEA3 is an accessible programming interface that allows users to submit gene sets for analysis and compares them to six separate databases of various types of ChIP experiments to identify the most likely active transcription factors yielding a given list. This analysis was conducted on both the top 300 genes per cluster and the gene list output from DE single to identify transcription factors driving both cell identity and cell fate change along a trajectory.

### 2.2.4 FeatureScatter Analysis for Gene Co-Expression

To demonstrate co-expression (or lack thereof) of genes in single cells within a cluster, SEURAT's FeatureScatter vignette was used, which allows any quantifiable parameter that can be retrieved from the matrix columns to be plotted in a scatter plot against any other feature. To achieve this, the MONOCLE3 cell data set (cds) file was converted to a SEURAT Object. Since the feature of interest was gene expression, Feature1 was set to one gene and Feature 2 to a second gene. The output was a scatter plot where each axis was the normalized expression of one of the genes, and each dot represents a single cell.

### 2.3 Immunofluorescent Staining and Confocal Imaging

### 2.3.1 Tissue Section Staining

Salivary glands intended for immunofluorescent staining were snap frozen as described above and embedded in O.C.T. compound (TISSUE-TEK. #4583). Using a LEICA CM1850 Cryostat, embedded tissues were cryosectioned into 10 μm cuts and mounted on SUPERFROST PLUS slides (FISHER SCIENTIFIC #12-550-15). Prior to staining. slides were washed in 1× PBS for 5 minutes to remove O.C.T compound, then overlaid with 4% paraformaldehyde for 10 minutes to fix. After fixation, slides were washed three times for 5 minutes, then overlaid in blocking solution containing 5% bovine serum albumin, 1% normal goat serum, and 0.1% TRITON-X and left to incubate at room temperature for 90 minutes. After blocking, slides were overlaid with primary antibody diluted in blocking solution and placed in a humidity box in 4 °C overnight. The next day, slides were washed three times in 1×PBS for 5 minutes and overlaid with ALEXAFLUOR-conjugated secondary antibodies (LIFE TECHNOLOGIES, 1:200) or preconjugated primary antibodies diluted in blocking solution according to manufacturer recommendation and incubated at room temperature for 75 minutes. Slides were then washed four times for 5 minutes and overlaid with DAPI diluted in 1×PBS and incubated at room temperature for 20 minutes. Slides were washed a final time in 1×PBS for 15 minutes, then mounted with VECTASHIELD Hardset Antifade Mounting Medium (VECTOR LABORATORIES #H-1400) and allowed to set overnight in the dark at 4 °C. After staining, slides were stored at 4 °C in the dark. Slides were imaged on an inverted NIKON ECLIPSE TI inverted microscope equipped with an AIR point scanning confocal system with alkali photomultiplier tubes for blue and far-red detection, and GaAsP photomultiplier tubes for green and red detection. Images were taken at 40×-60× magnification and 1024×1024 resolution and processed using NIS Elements Advanced Research imaging software (Version 5.11.01) and FIJI IMAGEJ (Version 2.0.0-rc-69/1.52p).

### 2.3.2 Organoid Staining

Organoids were fixed by mixing 8% paraformaldehyde (PFA) 1:1 into organoid culture media and incubating for 15 minutes at room temperature. PFA was removed and organoids were washed three times for 10 minutes in 1× phosphate-buffered saline (PBS) while nutating. To stain, organoids were blocked overnight at 4 °C in buffer containing 5% normal goat serum and 0.3% TRITON-X 100 in PBS. Cells were incubated overnight at 4 °C with primary antibodies diluted in antibody dilution buffer containing 1% BSA and 0.3% TRITON-X 100 in PBS. The next day, organoids were washed 3×5 minutes in 1×PBS while nutating at room temperature and were then incubated overnight at 4 °C with ALEXAFLUOR-conjugated secondary antibodies (1:200) and 0.1 mg/mL 4′,6-diamidino-2-phenylindole (DAPI) nuclear counterstain diluted in the above antibody dilution buffer. The next day, organoids were washed 3×5 minutes in 1×PBS while nutating at room temperature and mounted in 3D well slides in VECTASHIELD Hardset Antifade Mounting Medium. Organoids were imaged as described above.

### 2.4 Cell Culture and Differentiation

### 2.4.1 Pluripotent Cell Culture

The human induced pluripotent stem cell (iPSC) line WTC-11 (CORIELL INSTITUTE, GM25256) were cultured on MATRIGEL growth factor-reduced basement membrane matrix (CORNING) in mTeSR™1 media (STEMCELL TECHNOLOGIES: medium was used according to manufacturer instructions; see e.g., cdn.stemcell.com/mnedia/files/pis/10000003789-PIS_05.pdf?_ga=2.222029009.836134404.1686215811-1920030582.1686215811). Media was changed daily and cell were passaged once cells reached ~70% confluence using ACCUTASE (STEM CELL TECHNOLOGIES), about once every 3-4 days. 2.4.2 Differentiation of Salivary, Gland Organoids

To begin differentiation, WTC-11 iPSCs were passaged, counted. and plated in COSTAR Ultra Low Attachment 6 well plates (CORNING) at a density of 31,250 cell/cm² (e.g.. 350,000 cells per well of a 6 well plate, 9.6 cm² surface area) in mTeSR™1 supplemented with 50 μM Y-27632 (CALBIOCHEM) for a period of 3 days to allow for cluster formation. After 3 days, iPSCs form multiple small aggregates, around 25-30 per well, with some limited cell death. The doubling time of pluripotent human cells can be about 18-20 hours, but when pluripotent cells are grown in dense conditions such doubling time can decrease. It is thus estimated that the number or cells per aggregate during their beginning pluripotent phase can be about 11,000-14.000 cells per aggregate.

On d0, cell clusters were then switched to EPICULT™-C Media (STEM CELL TECHNOLOGIES: medium was used according to manufacturer instructions: see e.g.. cdn.stemcell.com/media/files/pis/29967-PIS_2_0_1.pdf?_ga=2.251883072.836134404.1686215811-1920030582.1686215811) supplemented with 400 nM Smoothened agonist (SAG) (SELLEKCHEM).

On d3 and d6, cells were fed with EPICULT™-C Media containing 400 nM SAG and 150 pM bone morphogenetic protein 4 (BMP4) (PEPROTECH). On d8, cells were fed with EPICULT™-C media supplemented with 1 mM SAG, 5 μM Chiron (MEDCHEM EXPRESS: also referred to herein interchangeably as Chir99021), 15 ng/mL epidermal growth factor (EGF) (PEPROTECH), 1 μM LDN-193189 (CAYMAN CHEMICAL) and 1 μM Neurotrophin-4 (FISHERSCI).

On d10, to push oral epithelium toward salivary epithelium, fresh d8 media was supplemented with 250 ng/nL fibroblast growth factor 10 (FGF10) (PEPROTECH).

On day 12 and d15, cells were fed with Dulbecco's Modified Eagle Medium/Nutrient Mixture F-12 (DMEM/F12) (GIBCO; medium was used according to manufacturer instructions) supplemented with 1×N2 supplement, 250 ng/mL FGF10, 125 ng/mL fibroblast growth factor 1 (FGF1) (PEPROTECH) and 125 ng/mL fibroblast growth factor 7 (FGF7) (PEPROTECH).

The components of DMEM/F12 basal medium include, but are not limited to: Amino Acids including but not limited to: Glycine, L-Alanine, L-Arginine hydrochloride, L-Asparagine-H₂O, L-Aspartic acid, L-Cysteine hydrochloride-H₂O, L-Cystine 2HCl, L-Glutamic Acid. L-Glutamine, L-Histidine hydrochloride-H₂O, L-Isoleucine, L-Leucine, L-Lysine hydrochloride, L-Methionine. L-Phenylalanine, L-Proline, L-Serine, L-Threonine, L-Tryptophan, L-Tyrosine disodium salt dihydrate, and/or L-Valine. and Vitamins including but not limited to: Biotin. Choline chloride, D-Calcium pantothenate, and/or Folic Acid (see e.g., thermofisher.com/us/en/home/technical-resources/media-formulation.55.html).

The components of N2 supplement include, but are not limited to: human transferrin; insulin recombinant full chain; progesterone; putrescine; and/or selenite (see e.g., thermofisher.com/us/en/home/teclmical-resources/media-fonnulation.166.html).

On d18 and d21, cells were fed with DMEM/F12™ supplemented with 1×N2, 250 ng/mL FGF10, 250 ng/mL Neuregulin 1 (NRG1) (PEPROTECH), 125 ng/mL FGF1, 125 ng/mL FGF7.1% laminin (FISHER SCI), and 3% GFR MATRIGEL (CORNING). Specifically, the MATRIGEL/laminin mixture (e.g.. 1% laminin (FISHER SCI), and 3% GFR MATRIGEL (CORNING) is added to the media on d18 and d24; these components form a light gel around the organoids (a form of embedding) that remains for the rest of the culture time, and the media is not supplemented with the MATRIGEL/laminin mixture following d24.

On d24, cells were fed with fed with the d18 media composition supplemented with 100 ng/mL EGF, 5 μM Chiron, 10 μM SB-431452 (MEDCHEM EXPRESS), and 50 μM Y-27632. Following d24 (e.g., up to and beyond d35-d75), cells were fed every 3-4 days with DMEM/F12™ supplemented with 1×N2, 250 ng/mL FGF10, 250 ng/mL NRG1, 125 ng/mL FGF1, 125 ng/mL FGF7, 100 ng/mL EGF, 5 μM Chiron, 10 μM SB-431452.

### 2.4.3 Diabetic Model

At d35, after organoids begin expressing Amylase Alpha 1A (AMY1A). At d50, organoids were overlaid with timepoint appropriate media supplemented with an additional 53 mM D-glucose (FISHER SCI) for the diabetic sample, or the equal amount of D-mannitol (FISHER SCI) in the control group as the osmotic control. Diabetic samples were also supplemented with 1 ng/ml each of cytokines tumour Necrosis Factor alpha (TNFa) and interleukin-6 (IL-6). Organoids were cultured in these conditions for a period of 1 week, then fixed as described above for analysis.

### 2.5 Mouse Kidney Capsule Implantation

Salivary gland organoids were differentiated using the protocol above for 35 days (after which cells begin to express AMY1A). On the day of transplantation, organoids were transferred to a beveled, kinked PE50 tubing [BD INTRAMEDIC, #427517]. Adult NOD-SCID mice (8-10 weeks old) were anesthetized using isoflurane supplemented with oxygen within an anesthesia chamber. Animals were then transferred to a nose cone and maintained on 2% isoflurane. To maintain a body temperature of 37 °C mice were placed on a heating pad throughout the transplantation. Mice were shaved, and the site was sterilized with betadine and alcohol wipes prior to making a 1-2 cm dorsal flank incision. Kidneys were externalized using a cotton swab, and the capsule was nicked near the caudal end using a needle tip (22 gauge). The beveled PE50 tubing was inserted beneath the capsule and cellular material was implanted under the control of a Hamilton syringe. A cotton swab was used to clot and seal the opening in the capsule to hold the implant in place. Next, the kidney was returned to the abdominal cavity, the peritoneum was sutured shut with absorbable sutures, and the skin was closed with surgical staples. Experiment was performed in compliance with ethical regulations. Mice were placed into a heated recovery cage until they regained consciousness, and then moved back to their home cage and transported back to the vivarium. 20 days after injection, mice were euthanized, kidneys were excised with graft intact and fixed in 4% paraformaldehyde at 4 °C for 1.25 hours and transferred to 30% sucrose solution at 4 °C overnight. Kidneys were then bisected and embedded in embedding cryo-molds [SAKURA, #25608-916] with TISSUE-TEK O.C.T. compound [SAKURA, #4583]. The embedded tissue was then frozen by placing a cold-resistant beaker of 2-methylbutane solution [EMD, #MX0760-1] into liquid nitrogen, which causes fast cooling to −80 °C. Samples were then placed in a −80 °C freezer for storage until cryo-sectioning. 10-20 μm-thick sections were made on pre-chilled SUPERFROST PLUS microscope slides [FISHERBRAND, #12-550-15] and then stored at −80 °C until used for immunofluorescence analysis.

### 2.6 Calcium Release Assay

D50 salivary gland organoids were digested with TRYPLE EXPRESS for 5 minutes at 37 °C and plated on 96-well flat bottomed plates coated in 5% MATRIGEL [CORNING, #3603] and allowed to attach for 24 hours. Cells were starved in low-glucose DMEM/F12 with 1×N2 supplement for 6 hours. Following starvation, the cells were incubated in media containing 5 μM CALBRYTE 520 AM fluorescent intracellular calcium indicator [AAT BIOQUEST, #20651] for 30 min at 37 °C. Cells were washed 3× with serum-free media and treated with 1 μM of the cholinergic agonist carbachol [CAYMAN CHEMICAL #14486]. Confocal live imaging was done on a NIKON YOKOGAWA W1 spinning disk confocal microscope using a 20× objective. Parameters for each live frame: Excitation/Emission filters for GFP fluorescence, Exposure time of 150 ms, Acquisition rate of 1 sec/frame, and total recording time of 5 minutes (20 second baseline recording+4.5 minutes min ligand treatment time). Images were processed with FIJI software distribution of IMAGEJ v1.52i and frame-by-frame cellular fluorescence intensity was tracked and quantified with CELLPROFILER. Dose-specific average calcium release was calculated by tracking each individual cell's response during the recording time and computing the mean peak fluorescence achieved by all cells in the frame. An average of 50-100 cells were tracked per recording.

### 2.7 Western Blotting and Coomassie Staining

Cells were lysed with lysis buffer containing 20 mM Tris-HCl [SIGMA-ALDRICH, #1185-53-1](pH 7.5), 150 mM NaCl, 15% Glycerol [SIGMA-ALDRICH, #G5516], 1% TRITON-X [SIGMA-ALDRICH, #9002-93-1], 3% SDS [SIGMA-ALDRICH, #151-21-3], 25 mM b-Glycerophosphate [SIGMA-ALDRICH, #50020-100G], 50 mM NaF [SIGMA-ALDRICH, #7681-49-4], 10 mM Sodium Pyrophosphate [SIGMA-ALDRICH, #13472-36-1], 0.5% Sodium Orthovanadate|SIGMA-ALDRICH, #13721-39-6|, 1% PMSF [ROCHE LIFE SCIENCES, #329-98-6], 25 U benzonase nuclease [EMD, #70664], protease inhibitor cocktail [PIERCE Protease Inhibitor Mini Tablets, THERMO SCIENTIFIC, #A32963], and phosphatase inhibitor cocktail 2 [SIGMA-ALDRICH, #P5726] in a tube. 4× Laemmli Sample Buffer [Bio-Rad, #1610747] containing 10% beta-mercaptoethanol [SIGMA-ALDRICH, #M7522-100] was added to the cell lysate and then heated at 95 °C for 10 min and stored at −80 °C.

Alternatively, cell culture media containing secreted vesicles was collected at each feed and concentrated using an AMICON Ultra Centrifugal filter with a 30 kDa molecular weight cutoff (MILLIPORE #UFC 9030), and concentrated protein was run through western blot protocol described below. Concentrated proteins in media were used immediately for experiment or stored at −20 °C.

For Western Blotting, protein samples were thawed over ice and then heated at 95 °C for 10 minutes. A 4-10% SDS-PAGE gel was loaded with 30 μL of protein per well and separated for 30 min at 250V. Proteins were transferred onto a nitrocellulose membrane for 12 minutes using the semi-dry turbo transfer Western blot apparatus [BIO-RAD]: the membrane was then blocked in 5% bovine serum albumin (BSA) [VWR, #0332-500G] for 1 hour. The membrane was incubated with AMY1A primary antibody on a rocker at 4 °C overnight. The next day, membranes were washed with 1× Tris-buffered saline with TWEEN (TBS-T) (3 times, 10 min intervals) and incubated with the respective anti-rabbit horseradish peroxidase (HRP) conjugated secondary antibody [BIORAD, #1706515](1:10,000 dilution in 5% BSA) at room temperature for 1 hour. Membranes were washed with 1×TBS-T (3 times, 10 min intervals) after secondary antibody incubation, developed using a Chemiluminescence developer, and imaged using a BIO-RAD CHEMIDOC Imager.

For Coomassie, following, protein gel was overlaid with 200 ml Coomassie Blue staining solution for 3 hours and then destained in 10% acetone 50% methanol for an additional 3 hours and imaged with the BIO-RAD CHEMIDOC Imager.

2.8 MITOSTRESS Measured with Seahorse Cellular Flux Assay

D50 salivary gland organoids were digested with TRYPLE EXPRESS for 5 minutes at 37 °C and plated on 96-well flat bottomed plates coated in 5% MATRIGEL [Corning, #3603] and allowed to attach for 24 hours. For the MITOSTRESS Assay in which the oxygen consumption rate (OCR) was measured, culture media were exchanged for base media (unbuffered DMEM (SIGMA D5030) supplemented with sodium pyruvate (GIBCO, 1 mM) and with 25-mM glucose I hour prior to the assay. Substrates and selective inhibitors were injected during the measurements to achieve final concentrations of glucose (2.5 mM), 4-(trifluoromethoxy) phenylhydrazone (FCCP, 500 nM), oligomycin (5 mM), antimycin (2.5 mM), rotenone (2.5 mM). The OCR values were normalized to the number of cells present in each well, quantified by the Hoechst staining (HO33342; SIGMA-ALDRICH). Changes in OCR in response to substrates and inhibitors addition were defined as the maximal change after the chemical injection compared to the last OCR value before the injection.

### 3. Exemplary Results

### 3.1 Cell Clustering and Pseudotime Analysis Reveal Three Distinct Developmental Trajectories for Major Tissues in the Salivary Gland.

In order to identify individual cell populations in the developing human fetal salivary gland development, single-cell combinatorial indexing RNA sequencing (sciRNAseq) was performed on human fetal submandibular salivary glands from three developmental timepoints: 12-13 weeks gestation, 14-16 weeks gestation, and 17-19 weeks gestation (see e.g., FIG. 1A-1B) (see e.g., Alghadeer et al., 2022. Alghadeer et al., 2023). Unbiased clustering using MONOCLE3 resulted in 14 unique clusters (see e.g., FIG. 1D) that were identifiable by molecular and functional markers. Cell clusters partitioned clearly along epithelial and non-epithelial lines (see e.g., FIG. 1D, left/right boxes). The large left island of clusters (left box) is comprised of the epithelial portions of the salivary gland, that is to say, the duct types and myoepithelial tissues, while the right side (right box) is comprised of support tissues of the salivary gland like the mesenchyme that surrounds the ducts, fibroblasts, and stromal cells, as well as endothelial, immune, and neuronal type cells.

To examine the epithelial developmental trajectory, the epithelial clusters were isolated and trajectory (see e.g., FIG. 1E) and pseudotime analysis (see e.g., FIG. 1F) were conducted, which predict the likely developmental trajectory of a given cell set through a biological process. These data predict that the earlier progenitor types (clusters 7 and 1) give rise to more mature cell types that appear at later developmental timepoints (clusters 2-6). This developmental prediction indicates that the major cells in the salivary epithelium arise through three distinct trajectories (see e.g., FIG. 1G), beginning with BEPs in cluster 7. BEP give rise to myoepithelium (cluster 3) and excretory duct (cluster 4), and, through a transitional cluster, to duct progenitors (cluster 1). DPs give rise to either ID (cluster 6) or SD (cluster 2). Finally, ID ultimately can give rise to the proacinar cells via distal tip duct reorganization (cluster 5) and some of the SD yields a stem cell-like population (cluster 9).

### 3.1.1 Striated-Intercalated Bifurcation Involves Differentially Regulated Wnt, TGFβ and Notch Signaling

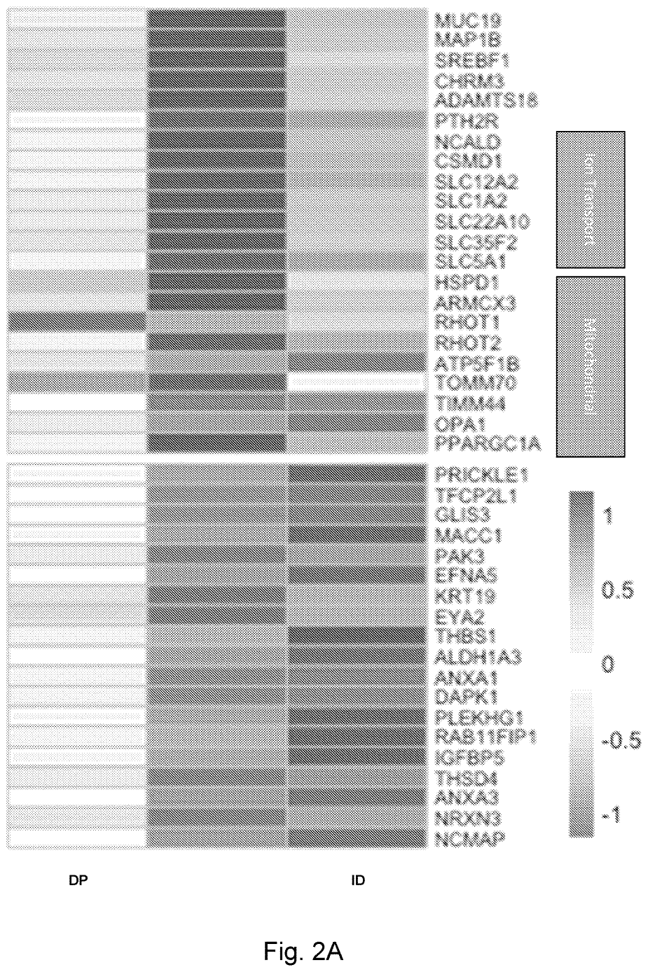

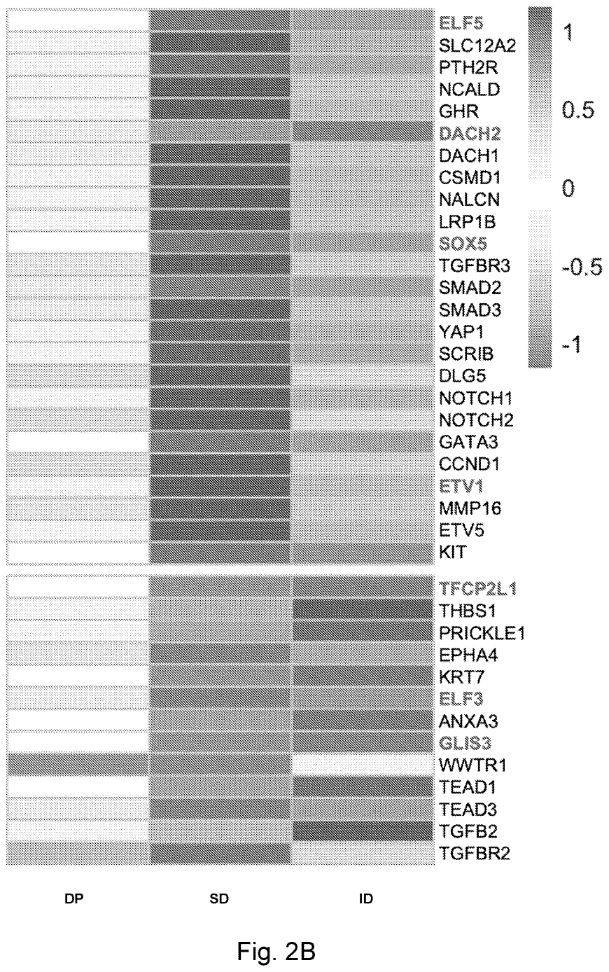

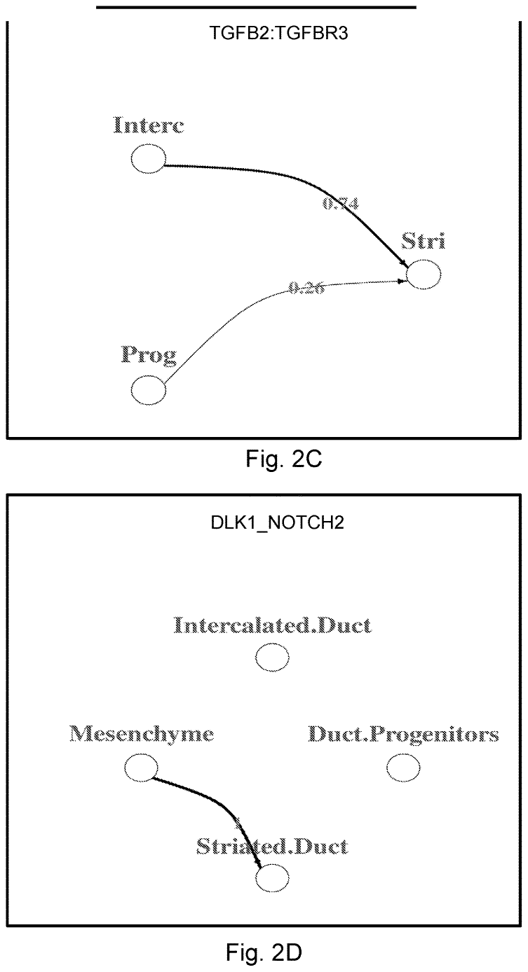

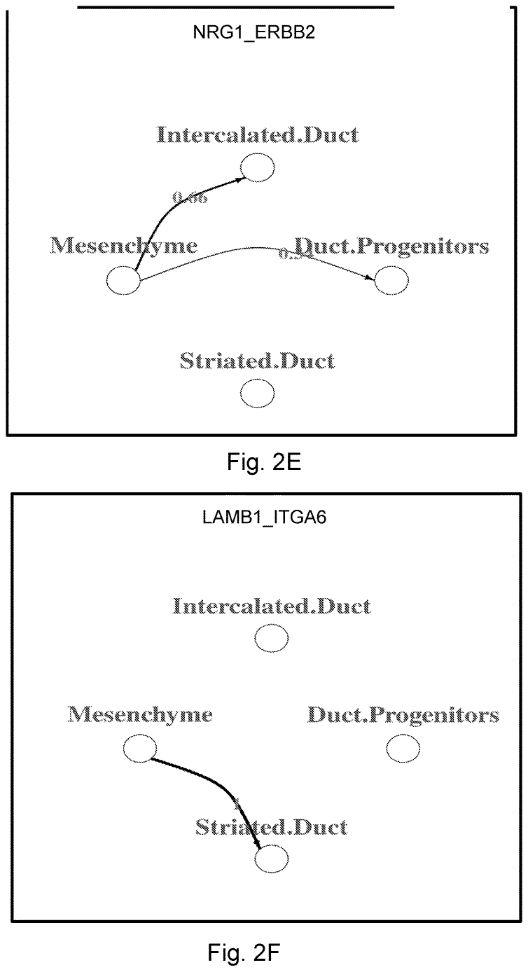

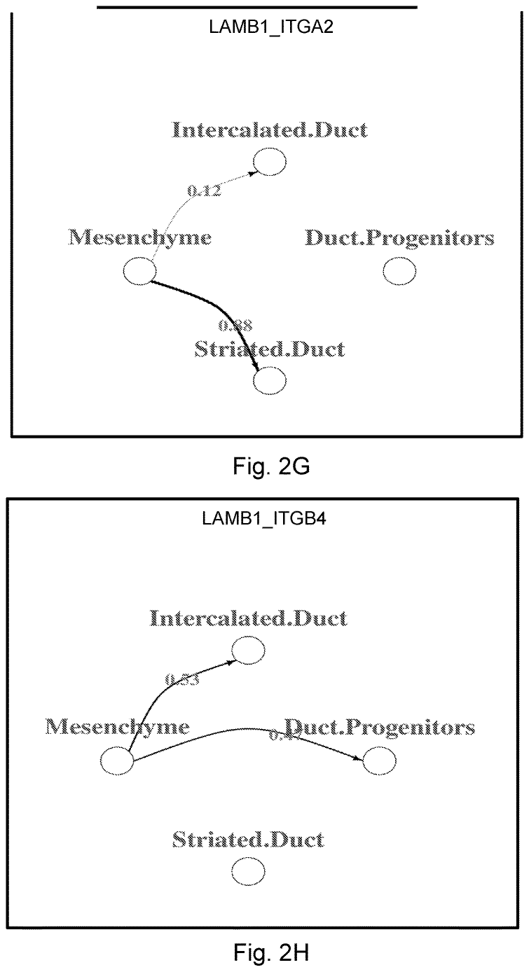

DP and ID express high levels of several genes that regulate Wnt (Wingless-related integration site) signaling, the most prominent of which is Prickle Planar Cell Polarity Protein 1 (PRICKLE1), a protein that activates non-canonical Wnt signaling to drive planar cell polarity and has also been shown to have context-dependent agonistic and antagonistic effects on canonical Wnt signaling. ChEA3 Transcription Faction prediction (see e.g., FIG. 2A, down-regulated genes) based on the differentially upregulated genes in the intercalated cluster predicted the activity of Wnt-related transcription factors with significant expression: the Catenin Beta 1 (CTNNB1) transcriptional cofactor Transcription Factor 7 Like 2 (TCF7L2), and the transcription factor CP2 Like 1 (TFCP2L1). Both transcription factors are responsible for the expression of multiple differentially expressed genes in ID cells (see e.g., FIG. 2A, black genes below corresponding down-regulated genes), but most interestingly, they drive expression of ID-specific genes Thrombospondin 1 (THBS1), PRICKLE1, Keratin 7 (KRT7), indicating that Wnt signaling can be involved in promoting the ID identity.

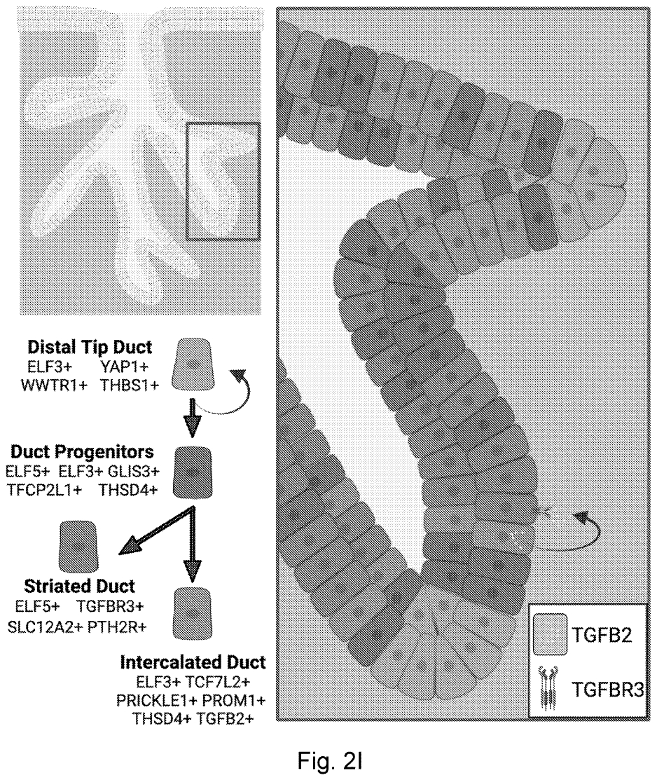

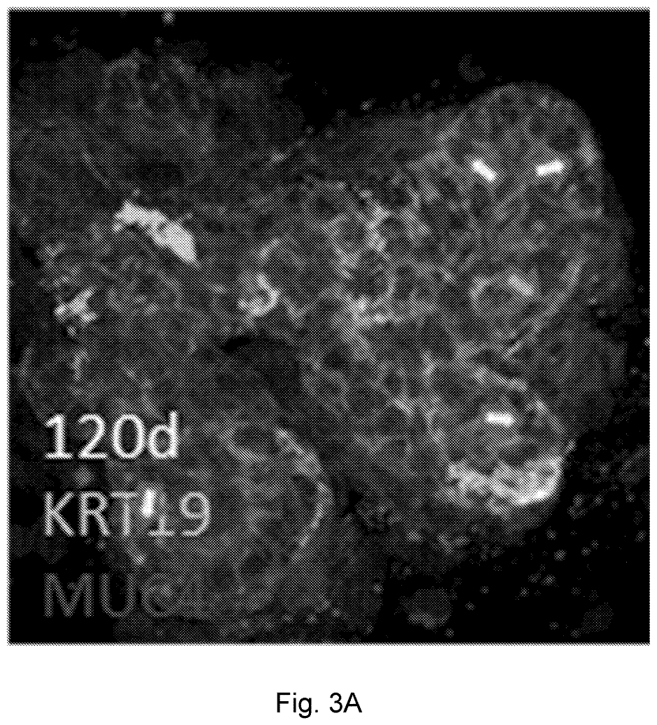

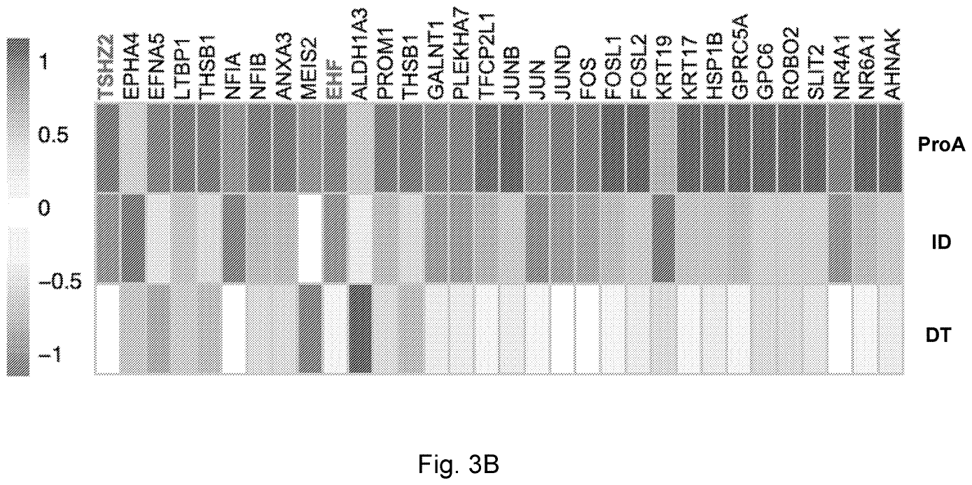

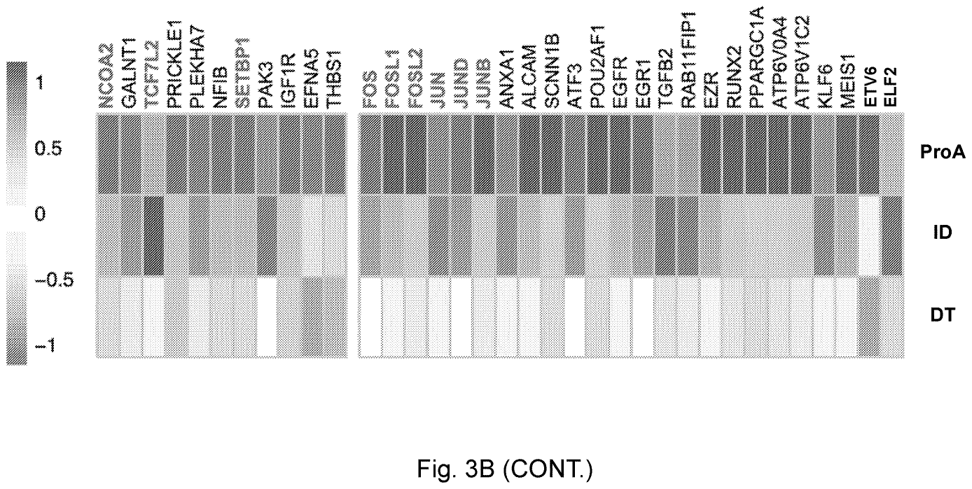

Ligand-receptor analysis on salivary epithelium and support tissues including mesenchyme indicate that a crosstalk takes place between ID and SD. Using a ligand receptor analysis program called talklr, which identifies significant interactions between cell clusters, a significant interaction was found between Transforming Growth Factor Beta 2 (TGFB2)-expressing ID and Transforming Growth Factor Beta Receptor 3 (TGFBR3)-expressing SD (see e.g., FIG. 2B). Interestingly, although ID expresses high levels of TGFB2 and one of its receptors, Transforming Growth Factor Beta Receptor 2 (TGFBR2), it also expresses high levels of a gene called Thrombospondin Type 1 Domain Containing 4 (THSD4) which has been shown to repress transforming growth factor beta (TGFβ), indicating that TGFβ signaling is not autocrine and can be inhibited to maintain intercalated identity. Moreover, ID also expresses several other secreted factors in addition to TGFB2 including Annexin A1 (ANXA1) and THBS1 that can promote TGFβ signaling through TGFBR2 and TGFBR3, indicating that secreted factors from ID can drive striated identity in neighboring cells that express TGFβ receptor. Furthermore, KEGG analysis of ID enriched genes supports this, showing that while ID express secreted factors, they do not exhibit intracellular indicators of active TGFβ signaling. Analysis also indicated that SD exhibited enrichment of Notch signaling mediators. SD exhibited enriched expression of neurogenic locus notch homolog protein 1 (NOTCH1) and neurogenic locus notch homolog protein 2 (NOTCH2), and talklr indicated that there was significant interaction between NOTCH2 in the SD and Notch ligand Delta-like homolog 1 (DLK1) expressed by the mesenchyme (see e.g., FIG. 3E). Moreover. NOTCH1 and NOTCH2 are both transcriptional targets of Sex Determining Region Y (SRY)-Box Transcription Factor 5 (SOX5). In some contexts, TGFβ has been shown to act synergistically with Notch, and SOX5 has been shown to be a DNA binding co-factor for Smads (Sma- and Mad- (Mothers Against Decapentaplegic) related proteins). Without wishing to be bound by theory, it is therefore hypothesized that after TGFβ activation occurs, SDs can maintain their identity by synergistic TGFβ and Notch regulation.

E74-Like Transcription Factor 3 (ELF3) and E74-Like Transcription Factor 5 (ELF5) are two of a family of epithelium specific Erythroblast Transformation Specific (ETS) transcription factors defined by their highly conserved ETS DNA binding domain. ELF3 was enriched in ID and DP, while ELF5 was enriched in SD, and ChEA3 analysis predicted them as responsible for several genes preferentially expressed by each cluster, indicating that these two factors can contribute to the ID-SD bifurcation. ELF3, which drives expression of highly expressed ID genes Annexin A3 (ANXA3), TGFB2, and duct marker Keratin 8 (KRT8), has been shown to drive ligand-independent transactivation of CTNNB1 in cancer models.

### 3.1.2 Cell-ECM Interactions Facilitate SD ID Bifurcation

The mesenchyme expresses a significant amount of Laminin Subunit Beta 1 (LAMB1), which binds to various integrins. Talklr indicates that LAMB1 interacts with different integrins to preferentially drive either SD or ID (see e.g., FIG. 2F-2H). LAMB1 interactions with Integrin Subunit Alpha 6 (ITGA6) (see e.g., FIG. 2F) and Integrin Subunit Alpha 2 (ITGA2) (see e.g., FIG. 2G) are predicted to drive SD, while its interaction with Integrin Subunit Beta 4 (ITGB4) (see e.g.. FIG. 2H) favors ID identity. Classically, laminin-integrin interactions drive epithelial cell polarity and migration, and mouse studies in the salivary gland have shown that LAMB1-ITGA6 interactions are involved in branching morphogenesis in early developmental; however, it is unclear how laminin-integrin signaling plays into this later-stage bifurcation. Several studies have shown that integrin-laminin signaling can affect expression of functional genes downstream. One interaction of particular interest found that ITGA6 was able to drive Notch signaling in endothelial cells during angiogenesis. consistent with the prediction that Notch signaling is enriched in the SD cluster, indicating laminin-integrin interactions can bolster intracellular signals that can bias DP towards a striated lineage, combining both cell-cell mediated signaling with secreted signaling.

Taken together, the data indicates that DPs are ELF3+ and ELF5+ ductal cells that bifurcate into ELF5+/Solute Carrier Family 12 Member 2 (SLC12A2)+SD and ELF3+/SLC12A2-ID. It also indicates that TGFB2 produced by ID drives TGFB signaling in SD and can help to drive this bifurcation (see e.g., FIG. 21).

### 3.2. Bioinformatic Analysis Introduces New Potential Regulators of Acinar Differentiation

A longstanding question in salivary gland development is what drives acinar differentiation. The tissues within the dataset are not yet mature enough to have mature acinar cells, therefore AMY1A and related salivary amylases, and other mature acinar markers like Muscle intestine stomach expression 1 (MIST1) are not yet expressed. Previous studies in mouse models have demonstrated that at some point in development, the distal tip of the duct reorganizes to produce acini. In humans, this process occurs comparatively slowly; salivary glands continue to develop through 28 weeks, at which time the acini begin to secrete products. However, maturation continues through postnatal stages, making studying their developmental trajectory difficult. However, tip reorganization has been observed as early as 16 weeks, indicating that the dataset is in an appropriate timeframe to evaluate the transition from distal tip duct to acinar cells. In the dataset, a small population of cells was observed that clustered along with the distal tip that expressed markers that have previously been observed in developing acinar tissues (Mucin 1 (MUC1), Mucin 4 (MUC4), Mucin 16 (MUC16)) that appears in the 17-19w timepoint (see e.g.. FIG. 1D, cluster 5. FIG. 3A). Although expression of mature acinar markers was not detected, the proacinar cells (tip of cluster 5) also uniquely express the related factor Basic Helix-Loop-Helix Family Member E40 (BHLHE40). MIST1, also called Basic Helix-Loop-Helix Family Member A15 (BHLHA15) has a predicted binding site for BHLHE40 is its promoter region, indicating that expression of BHLHA15 is imminent. Moreover, other genes that have been classically associated with mature acini exhibit non-acinar expression patterns in the developing human glands. Mucin 5B (MUC5B) is broadly expressed in ductal clusters (1,2,5,6,9), while Mucin 7 (MUC7) expression is more restricted, occupying clusters 1, 2, and 6 (duct progenitors, striated and intercalated duct, respectively). Aquaporin 5 (AQP5) expression is low but is primarily restricted to the striated duct (cluster 2). a phenomenon that has previously been observed in developing mouse tissues.

Consistent with the observation of MUC4 in proacinar cells, limited MUC4+ cells were observed in tissues 19 weeks and older (see e.g., FIG. 3A). Because ID shares significant gene expression with distal tip duct, ID along with distal tip and proacinar were evaluated to better highlight genes within the distal tip group that are involved with reorganization toward proacinar groups. Kyoto Encyclopedia of Genes and Genomes (KEGG) analysis of the proacinar group produced salivary secretion as the top function of that group. while gene ontology analysis ranked terms including glandular development, fluid homeostasis, and various metabolic responses among the top terms associated with the proacinar group. The gene ontology of the distal tip was much more general, with top ranked terms including growth, anatomical structure morphogenesis. Differential expression analysis and ChEA3 transcription factor prediction analysis (see e.g.. FIG. 3B) confirmed that distal tip duct and ID exhibit significant transcriptional overlap, except for two genes that are much more highly expressed in distal tip duct compared to ID. The first one, Myeloid Ecotropic Viral Integration Site 1 (Meis1) Homolog 2 (MEIS2), has been shown to be highly expressed in salivary glands and previous studies have shown that Meis2^{-/-} mice exhibit severe craniofacial malformations, including absent or underdeveloped salivary glands in at least 33% of the animals surveyed. The other, Aldehyde Dehydrogenase 1 Family Member A3 (ALDH1A3), a nicotinamide adenine dinucleotide (NAD)-dependent aldehyde dehydrogenase that catalyzes the formation of retinoic acid. Previous studies have found that expression of retinoic acid responsive genes arise earlier than most salivary gland-specific genes and persists throughout development, consistent with the ongoing presence of the distal tip as the gland continues to develop.

ChEA3 analysis revealed that two transcription factors were likely responsible for the bulk of the high gene expression in the distal tip. The first, Teashirt Zinc Finger Homeobox 2 (TSHZ2), was responsible for expression of ephrin receptor tyrosine kinase (RTK) Ephrin Type-A Receptor 4 (EPHA4) and its ligand Ephrin A5 (EFNA5), among others. Overexpressing TSHZ2 in mammary glands in mice accelerated their development while preventing malignancy. indicating that this factor can help to regulate the ongoing growth of the distal tip. The other transcription predicted transcription factor was ETS Homologous Factor (EHF), an epithelial-specific Ets-family transcription factor. This gene is not only responsible for the transcription of genes that are unique to that cluster, but also genes that are expressed both in the distal tip and then increase in the proacinar group. Most intriguing among these is a host of Activator protein 1 (AP-1) related genes: JUN (V-Jun Sarcoma Virus 17 Oncogene Homolog), FOS (FBJ (Finkel-Biskis-Jinkins) Murine Osteosarcoma Viral (V-Fos) Oncogene Homolog), etc. Among the salivary epithelium, these genes are highly enriched in the proacinar group. A recent study discovered that EHF exhibits anti-cooperative DNA binding with AP-1 factors; once EHF drives expression of AP-1 factors, the combination of FOS/JUN factors exhibits a conformation that preferentially excludes EHF from binding to downstream targets, but is compatible with others, driving a differential regulation of Ets-responsive genes. Incidentally, the proacinar group exhibits high expression of two other Ets family transcription factors: ETS Variant Transcription Factor 6 (ETV6) and E74-Like Transcription Factor 2 (ELF2), indicating that these factors are involved in specifying proacinar cells from the distal tip. As transcription factors, AP-1 components can be driven by multiple upstream pathways, but KEGG analysis of the distal tip group indicates that in salivary gland, this upstream driver can be ERBB (erythroblastic leukemia viral oncogene homologue) signaling mediated though Erb-B2 Receptor Tyrosine Kinase 4 (ERBB4) and Epidermal Growth Factor Receptor (EGFR). Previous studies have implicated WNT signaling and the transcription factor SRY-Box Transcription Factor 2 (Sox2) in acinar differentiation, both of which have also been shown to drive AP-1 signaling: without wishing to be bound by theory, it is hypothesized that AP-1 signaling is the link.

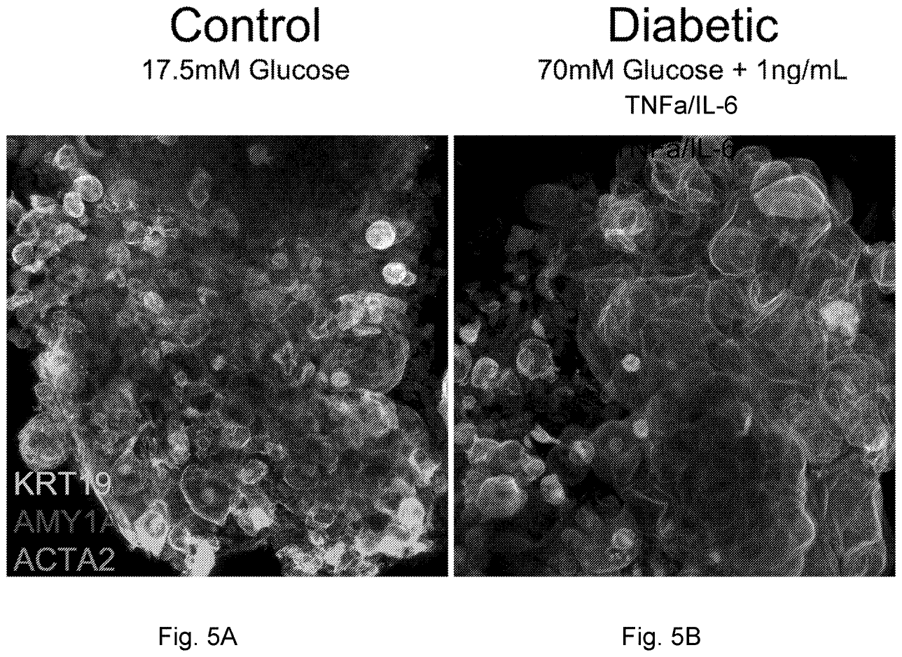

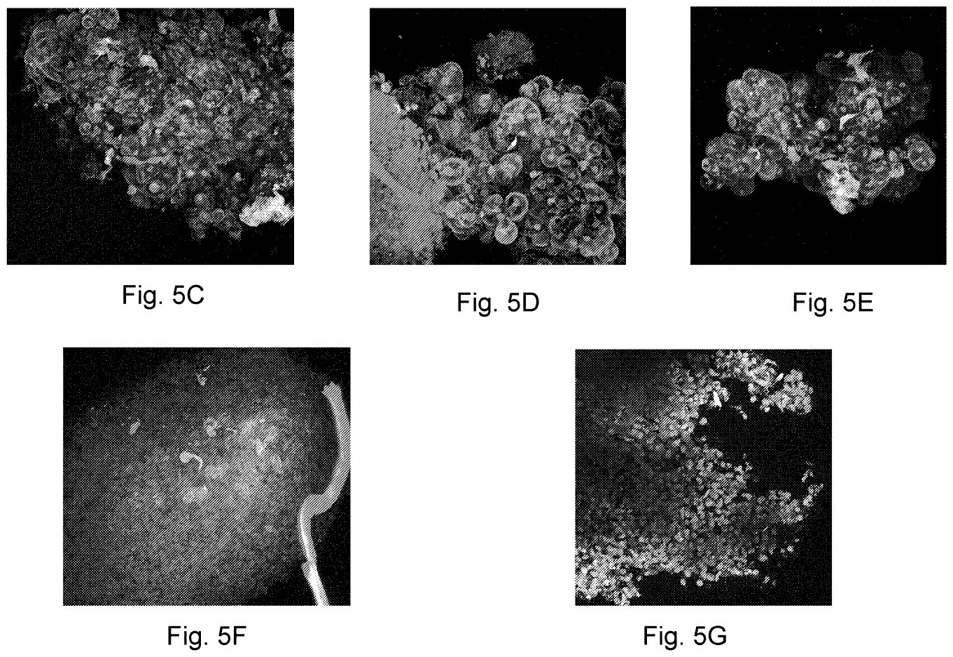

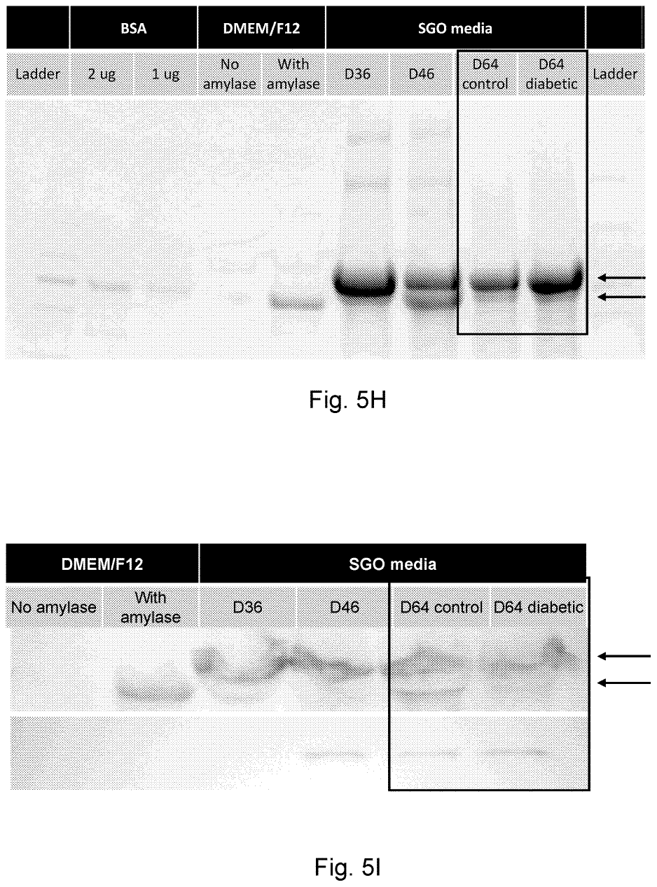

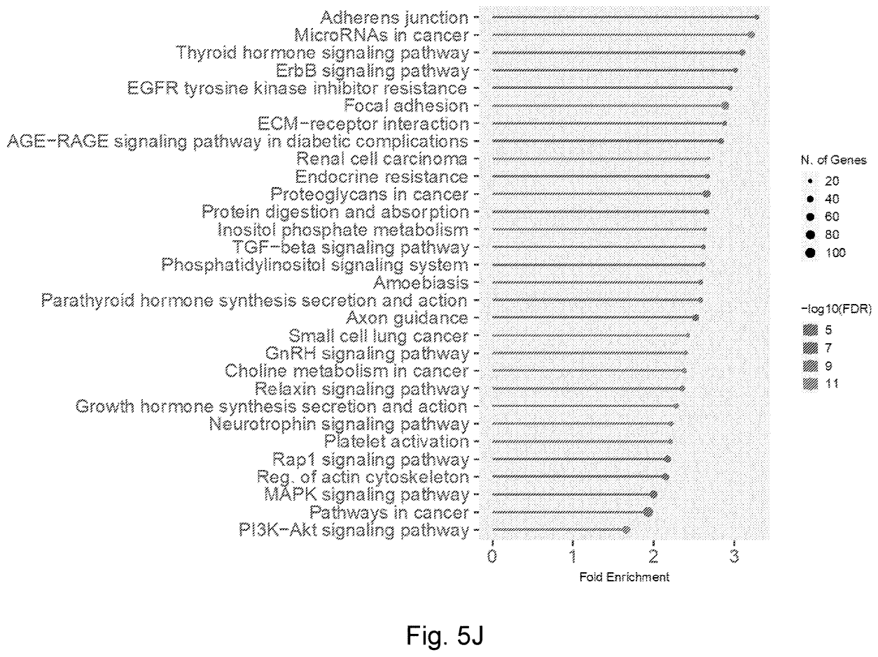

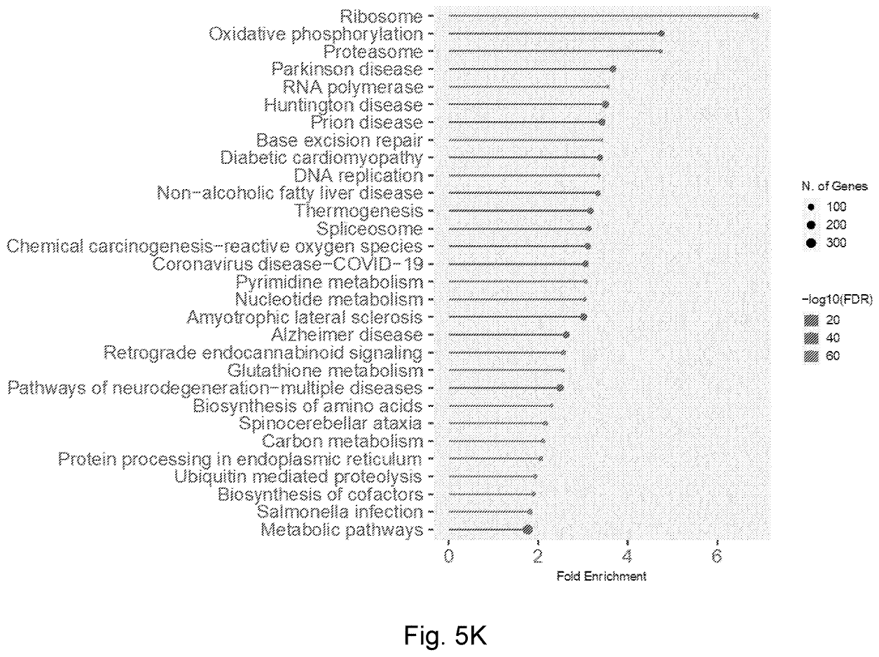

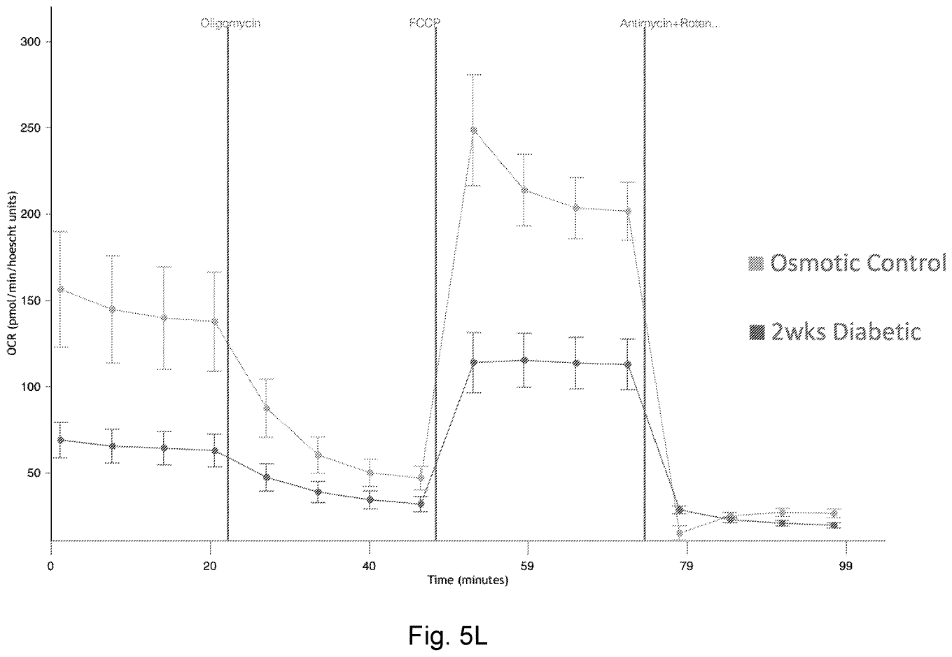

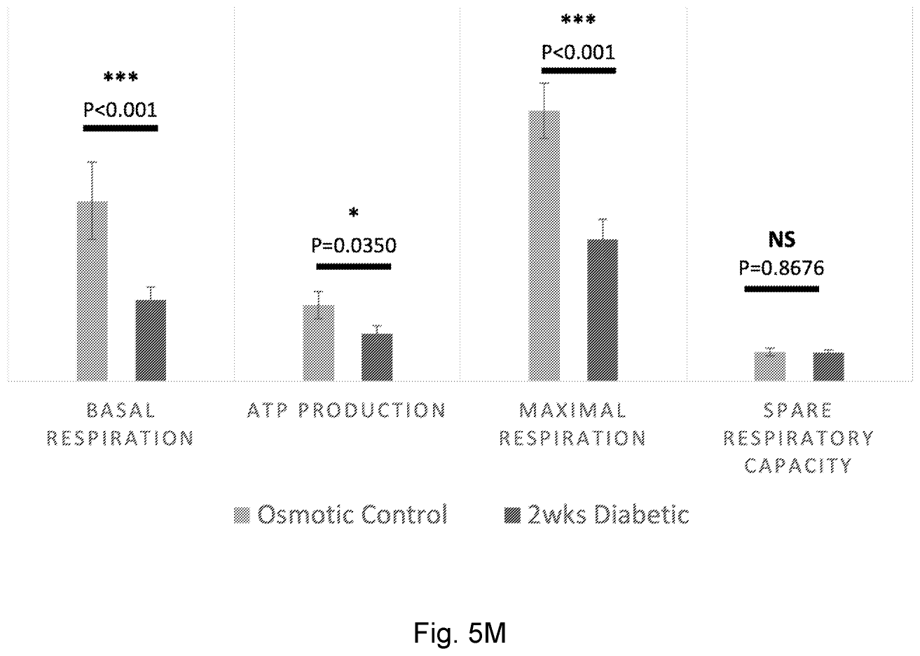

Together, these studies have added some clarity to human fetal salivary gland development and laid a foundation toward further in vitro developmental studies. At 12-13w, salivary glands are in the early pseudoglandular stage, comprised primarily of non-lumenized ducts with progenitor identity. In the 14-16w timepoint, tissues move into the late pseudoglandular phase, exhibiting more branching and lumenization, and by 17 weeks, cells have entered the canalicular stage, wherein distinct branching, widespread lumenization, and defined ductal types were observed. By 19 weeks, proacinar cells began to be observed, and branching became more complex and defined through 22 weeks. Genes that mark cell groups at each stage were also identified, which can be used as benchmarks for in vitro studies (see e.g., FIG. 5).

The knowledge acquired from this study was used to develop the methods for the hiPSC-derived salivary gland organoids, as described further herein.

### REFERENCES

The following references are incorporated herein by reference in their entireties.
1. Wang, J. & Laurie, G. W. Organogenesis of the exocrine gland. Dev Biol 273, 1-22 (2004).
2. Wells, K. L. et al. Dynamic relationship of the epithelium and mesenchyme during salivary gland initiation: the role of Fgf10. Biol Open 2, 981-9 (2013).
3. Garg, A. & Zhang, X. Lacrimal gland development: From signaling interactions to regenerative medicine. Dev Dynam 246, 970-980 (2017).
4. Wansbury, O. et al. Transcriptome analysis of embryonic mammary cells reveals insights into mammary lineage establishment. Breast Cancer Res Bcr 13, R79-R79 (2011).
5. Cunha, G. R. et al. Normal and abnormal development of the male urogenital tract. Role of androgens, mesenchymal-epithelial interactions, and growth factors. J Androl 13, 465-75 (1992).
6. Pispa, J. & Thesleff, I. Mechanisms of ectodermal organogenesis. Dev Biol 262, 195-205 (2003).
7. Shih, H. P., Panlasigui, D., Cirulli, V. & Sander, M. ECM Signaling Regulates Collective Cellular Dynamics to Control Pancreas Branching Morphogenesis. Cell Reports 14, 169-179 (2016).
8. Riopel, M. M., Li. J., Liu, S., Leask, A. & Wang, R. I integrin-extracellular matrix interactions are essential for maintaining exocrine pancreas architecture and function. Lab Invest 93. 31-40 (2013).
9. Wang, S.. Matsumoto. K., Lish, S. R., Cartagena-Rivera, A. X. & Yamada. K. M. Budding epithelial morphogenesis driven by cell-matrix versus cell-cell adhesion. Cell 184, 3702-3716.e30 (2021).
10. Entesarian, M. et al. FGF10 missense mutations in aplasia of lacrimal and salivary glands (ALSG). Eur J Hum Genet 15, 379-382 (2007).
11. Seymen, F. et al. Novel FGF10 mutation in autosomal dominant aplasia of lacrimal and salivary glands. Clin Oral Invest 21, 167-172 (2017).
12. Rivetti, S., Chen, C., Chen, C. & Bellusci, S. Fgf10/Fgfr2b Signaling in Mammary Gland Development, Homeostasis, and Cancer. Frontiers Cell Dev Biology 8, 415 (2020).
13. Donjacour, A. A., Thomson, A. A. & Cunha, G. R. FGF-10 plays an essential role in the growth of the fetal prostate. Dev Biol 261, 39-54 (2003).
14. May, A. J., Teshima, T. H. N., Noble, A. & Tucker, A. S. FGF10 is an essential regulator of tracheal submucosal gland morphogenesis. Dev Biol 451, 158-166 (2019).
15. Amerongen, A. N. & Veennan, E. Saliva—the defender of the oral cavity. Oral Dis 8, 12-22 (2002).
16. Marsh, P. D., Do, T., Beighton, D. & Devine, D. A. Influence of saliva on the oral microbiota. Periodontol 2000 70, 80-92 (2016).
17. Huang, N. et al. SARS-CoV-2 infection of the oral cavity and saliva. Nat Med 27, 892-903 (2021).
18. Song, J. et al. Systematic Analysis of ACE2 and TMPRSS2 Expression in Salivary Glands Reveals Underlying Transmission Mechanism Caused by SARS-CoV-2. J Med Virol 92, 10.1002/jmv.26045 (2020).
19. Xu, J.. Li, Y., Gan. F., Du, Y. & Yao, Y. Salivary Glands: Potential Reservoirs for COVID-19 Asymptomatic Infection. J Dent Res 99, 989-989 (2020).
20. Pedersen, A. et al. Primary Sjögren's syndrome: salivary gland function and clinical oral findings. Oral Dis 5, 128-138 (1999).
21. Pedersen, A., Sorensen, C., Proctor, G. & Carpenter, G. Salivary functions in mastication, taste and textural perception, swallowing and initial digestion. Oral Dis 24, 1399-1416 (2018).
22. Escobar, A. & Aitken-Saavedra, J. P. Salivary Glands—New Approaches in Diagnostics and Treatment. (2019) doi:10.5772/intechopen.72307.
23. Hamilton, S. N. et al. A cross-sectional assessment of long-term effects in adolescent and young adult head and neck cancer survivors treated with radiotherapy. J Cancer Surviv 1-10 (2021) doi:10.1007/s11764-021-01103-w.
24. Emmerson, E. & Knox, S. M. Salivary gland stem cells: A review of development, regeneration and cancer. Genesis 56, e23211 (2018).
25. Paula, F. de et al. Overview of Human Salivary Glands: Highlights of Morphology and Developing Processes. Anatomical Rec 300, 1180-1188 (2017).
26. Patel, V. N. & Hoffman, M. P. Salivary gland development: a template for regeneration. Seminars in cell & developmental biology 25-26, 52-60 (2014).
27. Porcheri, C. & Mitsiadis, T. A. Physiology, Pathology and Regeneration of Salivary Glands. Cells 8, 976 (2019).
28. Chibly, A. M., Aure, M. H., Patel, V. N. & Hoffman, M. P. Salivary gland function, development, and regeneration. Physiol Rev 102, 1495-1552 (2022).
29. May, A. J., Chatzeli, L., Proctor, G. B. & Tucker, A. S. Salivary Gland Dysplasia in Fgf10 Heterozygous Mice: A New Mouse Model of Xerostomia. Curr Med Chem 15, 674-682 (2016).
30. Lourengo, S. V. & Kapas, S. Integrin expression in developing human salivary glands. Histochem Cell Biol 124, 391 (2005).
31. Patel, V. N., Rebustini, I. T. & Hoffman, M. P. Salivary gland branching morphogenesis. Differentiation 74, 349-364 (2006).
32. Patel, N., Sharpe, P. T. & Miletich, I. Coordination of epithelial branching and salivary gland lumen formation by Wnt and FGF signals. Dev Biol 358, 156-167 (2011).
33. Aure, M. H., Symonds, J. M., Mays, J. W. & Hoffman, M. P. Epithelial Cell Lineage and Signaling in Murine Salivary Glands. J Dent Res 98, 1186-1194 (2019).
34. Miyazaki, Y., Nakanishi, Y. & Hieda, Y. Tissue interaction mediated by neuregulin-1 and ErbB receptors regulates epithelial morphogenesis of mouse embryonic submandibular gland. Dev Dynam 230, 591-596 (2004).
35. Knox, S. M. et al. Parasympathetic Innervation Maintains Epithelial Progenitor Cells During Salivary Organogenesis. Science 329, 1645-1647 (2010).
36. Dyachuk, V. et al. Parasympathetic neurons originate from nerve-associated peripheral glial progenitors. Science 345, 82-87 (2014).
37. Nitta, M., Kume, T. & Nogawa, H. FGF alters epithelial competence for EGF at the initiation of branching morphogenesis of mouse submandibular gland. Dev Dynam 238, 315-323 (2009).
38. Tang, C. S.-M. et al. Mutations in the NRG1 gene are associated with Hirschsprung disease. Hum Genet 131, 67-76 (2012).
39. Guo, L., Yu, Q. C. & Fuchs, E. Targeting expression of keratinocyte growth factor to keratinocytes elicits striking changes in epithelial differentiation in transgenic mice. Embo J 12, 973-986 (1993).
40. Knosp, W. M. et al. Submandibular Parasympathetic Gangliogenesis Requires Sprouty-Dependent Wnt Signals from Epithelial Progenitors. Dev Cell 32, 667-677 (2015).
41. Makarenkova, H. P. et al. Differential Interactions of FGFs with Heparan Sulfate Control Gradient Formation and Branching Morphogenesis. Sci Signal 2, ra55-ra55 (2009).
42. Yamaguchi, Y., Yonemura. S. & Takada, S. Grainyhead-related transcription factor is required for duct maturation in the salivary gland and the kidney of the mouse. Development 133, 4737-4748 (2006).
43. Hauser, B. R. et al. Generation of a Single-Cell RNAseq Atlas of Murine Salivary Gland Development. Iscience 23, 101838 (2020).
44. Sekiguchi, R., Martin, D., Core, G. and C. B. & Yamada, K. M. Single-Cell RNA-seq Identifies Cell Diversity in Embryonic Salivary Glands. J Dent Res 99, 002203451988388 (2019).
45. Song, E.-A. C. et al. Genetic and scRNA-seq Analysis Reveals Distinct Cell Populations that Contribute to Salivary Gland Development and Maintenance. Sci Rep-uk 8, 14043 (2018).
46. Medvinsky, A. & Livesey, F. J. On human development: lessons from stem cell systems. Development 142, 17-20 (2014).
47. Stuart, T. et al. Comprehensive Integration of Single-Cell Data. Cell 177, 1888-1902.e21 (2019).
48. Qiu, X. et al. Reversed graph embedding resolves complex single-cell trajectories. Nat Methods 14, 979-982 (2017).
49. Cao, J. et al. The single cell transcriptional landscape of mammalian organogenesis. Nature 566, 496-502 (2019).
50. Trapnell, C. et al. The dynamics and regulators of cell fate decisions are revealed by pseudotemporal ordering of single cells. Nat Biotechnol 32, 381-6 (2014).
51. Miao, Z., Deng, K., Wang, X. & Zhang, X. DEsingle for detecting three types of differential expression in single-cell RNA-seq data. Bioinformatics 34, 3223-3224 (2018).
52. Keenan, A. B. et al. ChEA3: transcription factor enrichment analysis by orthogonal omics integration. Nucleic Acids Res 47, W212-W224 (2019).
53. Hao, Y. et al. Integrated analysis of multimodal single-cell data. Cell 184, 3573-3587.e29 (2021).
54. Schindelin, J. et al. Fiji: an open-source platform for biological-image analysis. Nat Methods 9, 676-682 (2012).
55. Kreitzer, F. R. et al. A robust method to derive functional neural crest cells from human pluripotent stem cells. Am J Stem Cells 2, 119-31 (2013).
56. Cao, J. et al. Comprehensive single-cell transcriptional profiling of a multicellular organism. Science 357, 661-667 (2017).
57. Tao, H. et al. Mouse prickle1, the homolog of a PCP gene, is essential for epiblast apical-basal polarity. Proc National Acad Sci 106, 14426-14431 (2009).
58. Gibbs, B. C. et al. Prickle1 mutation causes planar cell polarity and directional cell migration defects associated with cardiac outflow tract anomalies and other structural birth defects. Biol Open 5, 323-335 (2016).
59. Wang, Y. talklr uncovers ligand-receptor mediated intercellular crosstalk. Biorxiv 2020.02.01.930602 (2020) doi:10.1101/2020.02.01.930602.
60. Liu, J. et al. ZNF37A promotes tumor metastasis through transcriptional control of THSD4/TGF-β axis in colorectal cancer. Oncogene 40, 3394-3407 (2021).
61. Atanasova, V. S. et al. Thrombospondin-1 Is a Major Activator of TGF-β Signaling in Recessive Dystrophic Epidermolysis Bullosa Fibroblasts. J Invest Dermatol 139, 1497-1505.e5 (2019).
62. Lin, H. Y. et al. The Soluble Exoplasmic Domain of the Type II Transforming Growth Factor (TGF)-β Receptor A HETEROGENEOUSLY GLYCOSYLATED PROTEIN WITH HIGH AFFINITY AND SELECTIVITY FOR TGF-β LIGANDS (*). J Biol Chem 270, 2747-2754 (1995).
63. Zhang, C. et al. WISP1 promotes bovine MDSC differentiation via recruitment of ANXA1 for the regulation of the TGF-β signalling pathway. Mol Cell Biochem 470, 215-227 (2020).
64. Pohlers, D. et al. Constitutive upregulation of the transforming growth factor-(pathway in rheumatoid arthritis synovial fibroblasts. Arthritis Res Ther 9, R59-R59 (2007).
65. Klüppel, M. & Wrana, J. L. Turning it up a Notch: cross-talk between TGFβ and Notch signaling. Bioessavs 27, 115-118 (2005).
66. Tang, Y. et al. Notch and Transforming Growth Factor-β (TGFβ) Signaling Pathways Cooperatively Regulate Vascular Smooth Muscle Cell Differentiation*. J Biol Chem 285, 17556-17563 (2010).
67. Nordin, K. & LaBonne, C. Sox5 Is a DNA-Binding Cofactor for BMP R-Smads that Directs Target Specificity during Patterning of the Early Ectodern. Dev Cell 31, 374-382 (2014).
68. Luk, I. Y., Reehorst, C. M. & Mariadason, J. M. ELF3, ELF5, EHF and SPDEF Transcription Factors in Tissue Homeostasis and Cancer. Molecules 23, 2191 (2018).
69. Wang, J.-L. et al. Elf3 drives β-catenin transactivation and associates with poor prognosis in colorectal cancer. Cell Death Dis 5, e1263 (2014).
70. Pandey, A. et al. Integrated genomic analysis reveals mutated ELF3 as a potential gallbladder cancer vaccine candidate. Nat Commun 11, 4225 (2020).
71. Belkin, A. M. & Stepp, M. A. Integrins as receptors for laminins. Microsc Res Techniq 51, 280-301 (2000).
72. Yazlovitskaya, E. M. et al. The laminin binding α3 and α6 integrins cooperate to promote epithelial cell adhesion and growth. Matrix Biol 77, 101-116 (2019).
73. Colburn, Z. T. & Jones, J. C. R. α6β4 Integrin Regulates the Collective Migration of Epithelial Cells. Am J Resp Cell Mol 56, 443-452 (2016).
74. Liu, F. & Wang, S. Molecular cues for development and regeneration of salivary glands. Histology and histopathology 29, 305-312 (2014).
75. Zhou, H. & Kramer, R. H. Integrin Engagement Differentially Modulates Epithelial Cell Motility by RhoA/ROCK and PAK1*. J Biol Chem 280, 10624-10635 (2005).
76. Xu, H., Pumiglia, K. & LaFlamme, S. E. Laminin-511 and α6 Integrins Regulate the Expression of CXCR4 to Promote Endothelial Morphogenesis. Biorxiv 846022 (2020) doi:10.1101/846022.
77. Chartier, N. T. et al. Laminin-5-integrin interaction signals through PI 3-kinase and RacIb to promote assembly of adherens junctions in HT-29 cells. J Cell Sci 119, 31-46 (2006).
78. Estrach, S. et al. Laminin-Binding Integrins Induce D114 Expression and Notch Signaling in Endothelial Cells. Circ Res 109, 172-182 (2011).
79. Holmberg, K. V. & Hoffman, M. P. Anatomy, biogenesis and regeneration of salivary glands. Monographs in oral science 24, 1-13 (2014).
80. Sadik, A. E., Mohamed, E. & Zainy, A. E. Postnatal changes in the development of rat submandibular glands in offspring of diabetic mothers: Biochemical, histological and ultrastructural study. Plos One 13, e0205372 (2018).
81. Larsen, H. S. et al. Localization of AQP5 during development of the mouse submandibular salivary gland. J Mol Histol 42, 71-81 (2011).
82. Lim, J.-Y. et al. Establishment and Characterization of Mesenchymal Stem Cell-Like Clonal Stem Cells from Mouse Salivary Glands. Tissue Eng Part C Methods 21, 447-457 (2015).
83. Sanz-Navarro, M. et al. Dental Epithelial Stem Cells Express the Developmental Regulator Meis1. Front Physiol 10, 249 (2019).
84. Fabik, J., Kovacova, K., Kozmik, Z. & Machon, O. Neural crest cells require Meis2 for patterning the mandibular arch via the Sonic hedgehog pathway. Biol Open 9, bio052043 (2020).
85. Moretti, A. et al. Crystal structure of human aldehyde dehydrogenase 1A3 complexed with NAD+ and retinoic acid. Sci Rep-uk 6, 35710 (2016).
86. Wright, D. M. et al. Retinoic acid regulates embryonic development of mammalian submandibular salivary glands. Dev Biol 407, 57-67 (2015).
87. Kania, A. & Klein, R. Mechanisms of ephrin-Eph signalling in development, physiology and disease. Nat Rev Mol Cell Bio 17, 240-256 (2016).
88. Uribe, M. L. et al. TSHZ2 is an EGF-regulated tumor suppressor that binds to the cytokinesis regulator PRC1 and inhibits metastasis. Sci Signal 14, eabe6156 (2021).
89. Madison, B. J. et al. Electrostatic repulsion causes anticooperative DNA binding between tumor suppressor ETS transcription factors and JUN-FOS at composite DNA sites. J Biol Chem 293, 18624-18635 (2018).
90. Emmerson, E. et al. SOX2 regulates acinar cell development in the salivary gland. Elife 6, e26620 (2017).
91. Pagin, M. et al. Sox2 controls neural stem cell self-renewal through a Fos-centered gene regulatory network. Stem Cells 39, 1107-1119 (2021).
92. Hankey, W. et al. Chromatin-associated APC regulates gene expression in collaboration with canonical WNT signaling and AP-1. Oncotarget 9, 31214-31230 (2018).

What is claimed is:

1. A composition comprising a salivary gland organoid culture.

2. The composition of claim 1, wherein the salivary gland organoid culture comprises salivary gland epithelial cells.

3. The composition of claim 2, wherein the salivary gland epithelial cells express acinar marker Amylase Alpha 1A (AMY1A), duct marker keratin 19 (KRT19), myoepithelial marker Actin Alpha 2 (ACTA2), and/or neuroepithelium marker Tubulin Beta 3 Class III (TUBB3).

4. The composition of claim 1, wherein the salivary gland organoid culture comprises salivary gland buds, branches, and/or lumenized ducts.

5. The composition of claim 1, wherein the salivary gland organoid culture is substantially free of endothelium and mesenchyme.

6. The composition of claim 1, wherein the salivary gland organoid culture is derived from pluripotent stem cells.

7. The composition of claim 1, wherein the salivary gland organoid culture can produce saliva.

8. The composition of claim 1, wherein the composition further comprises a salivary gland organoid culture medium comprising:
a) a medium supplement comprising:
i) transferrin;
ii) insulin;
iii) progesterone;
iv) putrescine; and/or
v) selenite;
b) at least one fibroblast growth factor;
c) a neuregulin;
d) an extracellular matrix glycoprotein;
e) a basement membrane matrix;
f) a growth factor for epithelium;
g) an inhibitor of a Glycogen Synthase Kinase (GSK) and/or activator of a Wingless/Integrated (Wnt) and β-catenin pathway; and/or
h) an inhibitor of transforming growth factor-β (TGF-β) type I receptor.

9. (canceled)

10. A method of establishing a salivary gland organoid culture, the method comprising:
a) culturing a population of pluripotent stem cells in aggregation medium for a sufficient time to promote formation of pluripotent stem cell clusters;
b) culturing the resultant pluripotent stem cell clusters in a salivary epithelium differentiation medium for a sufficient time to promote differentiation into salivary epithelium; and
c) culturing the resultant salivary epithelium in a salivary gland organoid differentiation medium for a sufficient time to promote differentiation into a salivary gland organoid culture.

11. The method of claim 10, wherein the pluripotent stem cells comprise induced pluripotent stem cells (iPSCs),
wherein the iPSCs are derived from a somatic cell sample from a subject,
wherein the subject has a disease or disorder that affects salivary gland function, and/or
wherein the iPSCs comprise cell line WTC-11.

12. (canceled)

13. (canceled)

14. (canceled)

15. The method of claim 10, wherein the aggregation medium comprises an inhibitor of Rho Associated Coiled-Coil Containing Protein Kinase (ROCK),
wherein the inhibitor of ROCK is Y-27632, and/or is at a concentration of about 50 μM.

16. (canceled)

17. (canceled)

18. (canceled)

19. (canceled)

20. The method of claim 10, wherein the salivary epithelium differentiation medium comprises at least one of the following:
a) an agonist of Smoothened from about day 0 to about day 8 after beginning the salivary epithelium differentiation medium, wherein the agonist of Smoothened is at a concentration of about 400 nM and/or is Smoothened Agonist (SAG);
b) a bone morphogenetic protein (BMP) from about day 3 to about day 8 after beginning the salivary epithelium differentiation medium, wherein the BMP is at a concentration of about 150 pM and/or is BMP4;
c) from about day 8 to about day 12 after beginning the salivary epithelium differentiation medium:
i) an agonist of Smoothened, wherein the agonist of Smoothened is at a concentration of about 1 mM and/or is SAG;
ii) an inhibitor of a GSK and/or activator of a Wnt and β-catenin pathway, wherein the inhibitor of a GSK and/or activator of the Wnt and β-catenin pathway is at a concentration of about 5 μM and/or is Chiron (Chir99021);
iii) a growth factor for epithelium, wherein the growth factor for epithelium at a concentration of about 15 ng/mL and/or is epidermal growth factor (EGF);
iv) an inhibitor of a BMP type I (BMP I) receptor, wherein the inhibitor of the BMP I receptor is at a concentration of about 1 μM and/or is LDN-193189; and/or
v) a neurotrophin, wherein the neurotrophin is at a concentration of about 1 μM and/or is Neurotrophin-4;
and/or
d) a fibroblast growth factor (FGF) from about day 10 to about day 12 after beginning the salivary epithelium differentiation medium, wherein the FGF is at a concentration of about 250 ng/mL and/or is FGF10.

21. (canceled)

22. (canceled)

23. (canceled)

24. (canceled)

25. (canceled)

26. The method of claim 10, wherein the salivary gland organoid differentiation medium comprises at least one of the following:
a) from about day 12 to about day 35-75 after beginning the salivary epithelium differentiation medium (or from about day 0 to about day 23-63 after beginning the salivary gland organoid differentiation medium):
i) a medium supplement comprising:
A) transferrin;
B) insulin;
C) progesterone;
D) putrescine; and/or
E) selenite; and/or
ii) at least one FGF;
b) a neuregulin from about day 18 to about day 35-75 after beginning the salivary epithelium differentiation medium (or from about day 6 to about day 23-63 after beginning the salivary gland organoid differentiation medium);
c) from about day 18 to about day 24-75 after beginning the salivary epithelium differentiation medium (or from about day 6 to about day 12-63 after beginning the salivary gland organoid differentiation medium):
i) an extracellular matrix glycoprotein; and/or
ii) a basement membrane matrix;
d) an inhibitor of Rho Associated Coiled-Coil Containing Protein Kinase (ROCK) from about day 24 to about day 27 after beginning the salivary epithelium differentiation medium (or from about day 12 to about day 15 after beginning the salivary gland organoid differentiation medium); and/or
e) from about day 24 to about day 35-75 after beginning the salivary epithelium differentiation medium (or from about day 12 to about day 23-63 after beginning the salivary gland organoid differentiation medium):
i) a growth factor for epithelium;
ii) an inhibitor of a GSK and/or activator of a Wnt and β-catenin pathway; and/or
iii) an inhibitor of TGF-β type I receptor.

27. The method of claim 26, wherein:
a) transferrin is at a concentration of about 10 mg/L;
b) insulin is at a concentration of about 5 mg/L;
c) progesterone is at a concentration of about 6.3 μg/L;
d) putrescine is at a concentration of about 16.11 mg/L;
e) selenite is at a concentration of about 5.2 μg/L;
f) the at least one FGF is at a concentration from about 125 ng/mL to about 250 ng/mL, and/or is selected from the group consisting of FGF10 at a concentration of about 250 ng/mL, FGF7 at a concentration of about 125 ng/mL, and FGF1 at a concentration of about 125 ng/mL;
g) the neuregulin is at a concentration of about 250 ng/mL, and/or is Neuregulin 1 (NRG1):
h) the extracellular matrix glycoprotein is at a concentration of about 1%, and/or is a laminin:
i) a basement membrane matrix at a concentration of about 3%, and/or is secreted by Engelbreth-Holm-Swarm (EHS) mouse sarcoma cells, and or the basement membrane matrix is growth factor reduced (GFR):
j) the inhibitor of ROCK is at a concentration of about 50 μM, and/or is Y-27632;
k) the growth factor for epithelium is at a concentration of about 100 ng/mL, and/or is epidermal growth factor (EGF);
l) the inhibitor of a GSK and/or activator of a Wnt and β-catenin pathway is at a concentration of about 5 μM, and/or is Chiron (Chir99021);
m) the inhibitor of TGF-β type I receptor is at a concentration of about 10 μM, and/or is SB-431452; and/or
n) the medium supplement is N-2 supplement.

28. (canceled)

29. (canceled)

30. (canceled)

31. (canceled)

32. The method of claim 10, wherein the sufficient amount of time to promote, formation of pluripotent stem cell clusters is about 3 days,
differentiation of pluripotent stem cell clusters into salivary epithelium is about 12 days,
differentiation of salivary epithelium into the salivary gland organoid culture is about 23-63 days or about 38 days,
differentiation of the population of pluripotent stem cells into the salivary gland organoid culture is about 53 days, and/or
differentiation of the pluripotent stem cell clusters into the salivary gland organoid culture is about 30-50 days or about 50 days.

33. (canceled)

34. (canceled)

35. (canceled)

36. (canceled)

37. The method of claim 10, wherein the salivary gland organoid culture secretes salivary amylase after about day 35 after beginning the salivary epithelium differentiation medium.

38. The method of claim 10, wherein the salivary gland organoid differentiation medium further comprises from at least about day 35 after beginning the salivary epithelium differentiation medium (or from at least about day 27 after beginning the salivary gland organoid differentiation medium):
a) a sugar or sugar alcohol,
wherein the sugar or sugar alcohol is at a concentration of about 53-70 mM,
the sugar is D-glucose, and/or
the sugar alcohol is D-mannitol; and/or
b) at least one proinflammatory cytokine,
wherein the at least one proinflammatory cytokine is at a concentration of about 1 ng/ml, and/or is TNFα and/or IL-6.

39. (canceled)

40. (canceled)

41. (canceled)

42. A salivary gland organoid culture prepared according to the method of claim 10.

43. (canceled)

44. (canceled)

45. (canceled)

46. (canceled)

47. (canceled)

48. (canceled)

49. (canceled)

50. A method of screening for an effective therapeutic for a salivary gland-associated disease, the method comprising:
a) exposing the salivary gland organoid culture of claim 1 to a potential therapeutic for a salivary gland-associated disease;
b) determining whether the salivary gland organoid culture exhibits decreased levels of a disease-associated marker for the salivary gland-associated disease or increased levels of a health-associated marker compared to the salivary gland organoid culture not exposed to the potential therapeutic; and
c) determining that the potential therapeutic is an effective therapeutic for the salivary gland-associated disease if the salivary gland organoid culture exhibits decreased levels of a disease-associated marker for the salivary gland-associated disease or increased levels of a health-associated marker compared to the salivary gland organoid culture not exposed to the potential therapeutic; or
d) determining that the potential therapeutic is not an effective therapeutic for the salivary gland-associated disease if the salivary gland organoid culture did not exhibit decreased levels of a disease-associated marker for the salivary gland-associated disease or increased levels of a health-associated marker compared to the salivary gland organoid culture not exposed to the potential therapeutic.

51. The method of claim 50, wherein the health-associated marker is selected from the group consisting of: Amylase Alpha 1A (AMY1A), keratin 19 (KRT19), and Actin Alpha 2 (ACTA2).
